(19) United States
(12) Patent Application Publication
(10) Pub. No.: US 2005/0154011 A1
(43) Pub. Date: Jul. 14, 2005

(54) Tetrahydroisoquinolnyl sulfamic acids

(75) Inventors: Sean Rees Klopfenstein, Loveland, OH (US); Matthew Brian Maier, Cincinnati, OH (US); David Robert Jones, Milford, OH (US); Jeffrey Lyle Gray, Loveland, OH (US); Matthew Eugene Pokross, Loveland, OH (US); Kevin Gene Peters, Loveland, OH (US); Artem Gennady Evdokimov, Loveland, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/633,744

(22) Filed: Aug. 4, 2003

Related U.S. Application Data
(60) Provisional application No. 60/455,977, filed on Mar. 18, 2003.
(60) Provisional application No. 60/448,749, filed on Feb. 20, 2003.

(51) Int. Cl. A61K 31/4709; C07D 41/02

(52) U.S. Cl. 514/310; 546/146

(57) ABSTRACT

Compounds of formula (I): 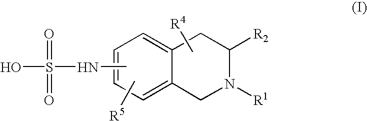 are effective in the treatment of protein tyrosine phosphatase (PTPase) mediated disorder such as diabetes.

### CROSS REFERENCE TO RELATED APPLICATIONS

This application claims priority under Title 35, United States Code 119(e) from Provisional Application Ser. No. 60/455,977 filed Mar. 18, 2003; Provisional Application Ser. No. 60/448,749 filed Feb. 20, 2003; and Provisional Application Ser. No. 60/406,829, filed Aug. 29, 2002, incorporated herein by reference.

### FIELD OF THE INVENTION

The present invention relates to (1,2,3,4-tetrahydro-isoquinolinyl)-sulfamic acids useful for the treatment of protein tyrosine phosphatase mediated disorders.

### BACKGROUND OF THE INVENTION

The regulation of protein tyrosine phosphorylation in vivo is mediated by the opposing actions of protein tyrosine kinases (PTKs) and protein tyrosine phosphatases (PTPases). The level of protein tyrosine phosphorylation of cellular proteins is determined by the balanced activities of PTKs, and PTPases (Hunter, Cell 80: 225-236 (1995)). When there is an imbalance of these activities, a disease state may arise. By logical extension, modulation of the tyrosine kinase/phosphatase balance could be used to treat diseases resulting from such imbalances.

For example, the mechanism of insulin action depends critically upon the phosphorylation of tyrosine residues in several proteins in the insulin signaling cascade. Enzymes that dephosphorylate these proteins, i.e., PTPases, are important regulators of insulin action. Therefore, the use of PTPase inhibitors may therapeutically enhance insulin action.

PTPases are implicated in the insulin receptor signaling pathway. Insulin is an important regulator of different metabolic processes and plays a key role in the control of blood glucose. Defects related to its synthesis or signaling lead to diabetes mellitus. Binding of insulin to its receptor causes rapid (auto)phosphorylation of several tyrosine residues in the intracellular part of the insulin receptor (beta subunit). Three closely positioned tyrosine residues (the tyrosine-1150 domain) must all be phosphorylated to obtain full activity of the insulin receptor tyrosine kinase (IRTK) which transmits the signal further downstream by tyrosine phosphorylation of other cellular substrates, including insulin receptor substrate-1 (IRS-1) (Wilden et al., J. Biol. Chem. 267: 16660-16668 (1992); Myers and White, Diabetes 42: 643-650 (1993); Lee and Pilch, Am. J. Physiol. 266: C319-C334 (1994); White et al., J. Biol. Chem. 263: 2969-2980 (1988)). The structural basis for the function of the tyrosine-triplet has been provided by recent X-ray crystallographic studies of IRTK that showed tyrosine-1150 to be autoinhibitory in its unphosphorylated state (Hubbard et al., Nature 372: 746-754 (1994)).

Several studies clearly indicate that the activity of the auto-phosphorylated IRTK can be reversed by dephosphorylation in vitro (reviewed in Goldstein, Receptor 3: 1-15 (1993); Mooney and Anderson, J. Biol. Chem. 264: 6850-6857 (1989)), with the tri-phosphorylated tyrosine-1150 domain being the most sensitive target for protein-tyrosine phosphatases (PTPases) as compared to the di- and mono-phosphorylated forms (King et al., Biochem. J. 275: 413-418 (1991)). It is, therefore, tempting to speculate that this tyrosine-triplet functions as a control switch of IRTK activity. Indeed, the IRTK appears to be tightly regulated by PTP-mediated dephosphorylation in vivo (Khan et al., J. Biol. Chem. 264: 12931-12940 (1989); Faure et al. J. Biol. Chem. 267: 11215-11221 (1992); Rothenberg et al, J. Biol. Chem. 266: 8302-8311 (1991)). The intimate coupling of PTPases to the insulin signaling pathway is further evidenced by the finding that insulin differentially regulates PTPase activity in rat hepatoma cells (Meyerovitch et al, Biochemistry 31: 10338-10344 (1992)) and in livers from alloxan diabetic rats (Boylan et al., J. Clin. Invest. 90: 174-179 (1992)). Further, when the strong PTPase-inhibitor pervanadate is added to whole cells an almost full insulin response can be obtained in adipocytes (Fantus et al., Biochemistry 28: 8864-8871 (1989); Eriksson et al., Diabetologia 39: 235-242 (1995)) and skeletal muscle (Leighton et al., Biochem J. 276: 289-292 (1991)). In view of the forgoing, there is a need to identify inhibitors of PTPase that are useful in a method of treating insulin receptor tyrosine kinase mediated disorders.

In another example, acid phosphatases/PTPases may be involved in negative regulation of osteoblast proliferation. Therefore, the use of the PTPase inhibitors may therapeutically enhance osteoblast proliferation and thereby treat bone disorders.

The rate of bone formation is determined by the number and the activity of osteoblasts, which in turn are determined by the rate of proliferation and differentiation of osteoblast progenitor cells. Histomorphometric studies indicate that the osteoblast number is the primary determinant of the rate of bone formation in humans (Gruber et al., Mineral Electrolyte Metab. 12: 246-254 (1987); reviewed in Lau et al., Biochem. J. 257: 23-36 (1989)). Acid phosphatases/PTPases may be involved in negative regulation of osteoblast proliferation. Thus, fluoride, which has phosphatase inhibitory activity, has been found to increase spinal bone density in osteoporotics by increasing osteoblast proliferation (Lau et al., supra). Consistent with this observation, an osteoblastic acid phosphatase with PTPase activity was found to be highly sensitive to mitogenic concentrations of fluoride (Lau et al., J. Biol. Chem. 260: 4653-4660 (1985); Lau et al., J. Biol. Chem. 262: 1389-1397 (1987); Lau et al., Adv. Protein Phosphatases 4: 165-198 (1987)). Interestingly, it was recently found that the level of membrane-bound PTPase activity was increased dramatically when the osteoblast-like cell line UMR 106.06 was grown on collagen type-I matrix compared to uncoated tissue culture plates. Since a significant increase in PTPase activity was observed in density-dependent growth arrested fibroblasts (Pallen and Tong, Proc. Natl. Acad. Sci. 88. 6996-7000 (1991)), it might be speculated that the increased PTPase activity directly inhibits cell growth. The mitogenic action of fluoride and other phosphatase inhibitors (molybdate and vanadate) may thus be explained by their inhibition of acid phosphatases/PTPases that negatively regulate the cell proliferation of osteoblasts. The complex nature of the involvement of PTPases in bone formation is further suggested by the recent identification of a novel parathyroid regulated, receptor-like PTPase, OST-PTP, expressed in bone and testis (Mauro et al. J. Biol. Chem. 269: 30659-30667 (1994)). OST-PTP is up-regulated following differentiation and matrix formation of primary osteoblasts and subsequently down-regulated in the osteoblasts which are actively mineralizing bone in culture. It may be hypothesized that PTPase inhibitors may prevent differentiation via inhibition of OST-PTP or other PTPases thereby leading to continued proliferation. This would be in agreement with the above-mentioned effects of fluoride and the observation that the tyrosine phosphatase inhibitor orthovanadate appears to enhance osteoblast proliferation and matrix formation (Lau et al., Endocrinology 116: 2463-2468 (1988)). In addition, it was recently observed that vanadate, vanadyl and pervanadate all increased the growth of the osteoblast-like cell line UMR106. (Cortizo et al., Mol. Cell. Biochem. 145: 97-102 (1995)).

In yet another example, the inhibition of acid phosphatases/PTPases may be involved in regulation of angiogenesis and tissue blood flow. Therefore, the use of PTPase inhibitors may be used to treat angiogenesis-mediated disorders.

Endothelial cells form the protective lining of blood vessels and respond to a variety of stimuli that modulate the form and function of the vasculature. Like the insulin receptor, the activity of endothelial PTKs is likely modulated by the action of endothelial PTPs. In support of this proposition, several PTPs have been shown to be expressed in endothelial cells (Fachinger et al. Oncogene 18: 5948-5953 (1999); Huang et al. J. Biol. Chem. 274: 38183-38188 (1999); Bianchi et al. Exp. Cell Res. 248: 329-338 (1999); Gaits et al. Biochem. J. 311: 97-103 (1995); Borges et al. Circ. Res. 79: 570-580 (1996)). One of these phosphatases, HCPTPA, has been shown to interact with and block the activation of a vascular endothelial growth factor (VEGF) receptor, VEGFR2, inhibiting VEGF-mediated downstream signaling and angiogenesis (Huang et al. J. Biol. Chem. 274: 38183-38188 (1999)). Another phosphatase, HPTPbeta, associates with and attenuates the activation of the receptor for angiopoietin 1 (Ang1) and angiopoietin 2 (Ang2), Tie2, (Fachinger et al. Oncogene 18: 5948-5953 (1999)). These studies indicate that targeting endothelial phosphatases will modulate the activation of endothelial PTKs and provide novel targets for therapeutic agents that modulate vascular form and function.

Abundant evidence demonstrates a role for multiple PTKs in the neovascularization of adult tissues. For example, inhibiting the action of VEGF or the angiopoietins inhibits tumor angiogenesis and limits tumor growth in animal models of cancer (Millauer et al. Cancer Res. 56: 1615-1620 (1996); Dias et al. Proc. Natl. Acad. of Sci. 98: 10857-10862 (2001); Lin et al. Proc. Natl. Acad. of Sci. 95: 8829-8834 (1998)). Conversely, administration of exogenous VEGF and/or Ang1 enhances the development of the collateral circulation and improves blood flow to ischemic tissue in animal models of occlusive cardiovascular disease (Witzenbichler et al. Am. J. of Pathol. 153: 381-394 (1998); Pearlman et al. Nature Medicine 10: 1085-1089 (1995); Banai et al. Circulation 89: 2183-2189 (1994); Shyu et al. 98: 2081-2087 (1998); Chae et al. Arteriosclero. Thromb. Vasc. Biol. 20: 2573-2578 (2000)). Taken together, these studies not only demonstrate a role for PTKs in neovascularization, but they also demonstrate that modulating the function of endothelial PTKs provides a novel therapeutic approach to modulation of angiogenesis and tissue blood flow in a broad range of diseases. Diseases in which enhanced vascular development would be beneficial include, but are not limited to, occlusive atherosclerotic cardiovascular disease, coronary artery disease, peripheral vascular disease, cerebrovascular disease (stroke), Berger's disease, diabetic vasculopathy and traumatic vascular damage. Diseases in which inhibition of neovascularization would be beneficial, include but are not limited to, cancer, arthritis, diabetic retinopathy, macular degeneration, psoriasis and endometriosis. In view of the foregoing, there is a need to identify inhibitors of PTPase that useful in a method of treating angiogenesis-mediated disorders.

In yet another example, the inhibition of acid phosphatases/PTPases may be involved in regulation of vascular tone. In addition to neovascularization and vascular remodeling, activation of PTKs can influence certain parameters of vascular function. Therefore, the use of PTPase inhibitors may be used to treat vascular tone mediated disorders.

Vascular tone is regulated by the endothelium and the endothelial factors that regulate vascular tone can be modulated by endothelial PTK signaling. Bolus infusion of VEGF induces a hypotensive response that is driven in part by VEGF-mediated activation of nitric oxide (NO) synthase and subsequent production by the endothelium of the potent vasorelaxant, nitric oxide (Hariawala et al. J Surgical Res. 63: 77-82 (1996); Ogasawara et al. Hypertension 39: 815-820 (2002)). Administration of fibroblast growth factor (FGF) has similar effects as VEGF on blood pressure that may also be mediated by enhanced endothelial nitric oxide production (Garcia-Calvo et al. Proc. Natl. Acad. Sci. 93: 11996-12001 (1996); Wu et al. Am. J. Physiol. 271: H1087-1093 (1996); Cuevas et al. Science 254: 1208-1210 (1991)). Thus, modulating PTK activation and downstream signaling provides a novel therapeutic approach to treating diseases characterized by alterations of vascular tone. Diseases which would benefit from decreased vascular tone include primary essential hypertension, secondary hypertension (i.e. renovascular or endocrine disorder mediated), pulmonary hypertension and portal hypertension. In view of the foregoing, there is a need to identify inhibitors of PTPase that are useful in a method of treating vascular tone mediated disorders.

In yet another example, the inhibition of acid phosphatases/PTPases may be involved in regulation of vascular permeability. Activation of endothelial PTKs has been shown to influence vascular permeability. Therefore, the use of PTPase inhibitors may be used to treat vascular permeability mediated disorders.

VEGF was originally isolated as a factor that increased vascular permeability (Senger et al. Cancer Metastasis Rev. 12: 303-324 (1993)). VEGF induced vascular permeability may be induced by the same high affinity receptor PTKs that mediate the other actions of VEGF i.e. angiogenesis and vasorelaxation (Gomez et al. Endocrinology 143: 4339-4348 (2002); Murohara et al. Circulation 97: 99-107 (1998)). In contrast to the permeabilizing effects of VEGF, Ang1 via its high affinity receptor, Tie2, blocks increases in vascular permeability by a variety of agents including VEGF (Thurston et al. Science 286: 2511-2514 (1999); Thurston et al. Nature Medicine 6: 460463 (2000)). These data demonstrate that activation and signaling by endothelial PTKs can either enhance or decrease vascular permeability and that approaches to specifically modulate endothelial PTK activation and signaling offers a novel therapeutic approach to pathologic states characterized by alterations in vessel leakiness. Diseases in which reducing vascular permeability would be beneficial include, but are not limited to, stroke, septic shock, burns, RDS (respiratory distress syndrome) and congestive heart failure. In view of the foregoing, there is a need to identify inhibitors of PTPase that are useful in a method of treating vascular permeability disorders.

In yet another example, the inhibition of acid phosphatases/PTPases may be involved in regulation of VEGF and thus the use of PTPase inhibitors may be used to treat VEGF-mediated disorders.

In addition to effecting the form and function of the vascular system directly, modulating the activity of signaling by endothelial PTKs has been shown to have indirect beneficial effects on other tissues. For example, decreasing the expression of VEGF in the myocardium results in the development of an ischemic cardiomyopathy (Carmeliet et al. Nat. Med. 5: 495-502 (1999)). Conversely, exogenous delivery of VEGF improves cardiac performance in animal models of heart failure and myocardial infarction (Suzuki et al. Circulation 104[suppl I]: I-207-1-212 (2001); Leotta et al. J. Thorac. Cardiovasc. Surg. 123: 1101-1113 (2002)). Increasing evidence indicates that VEGF can directly and indirectly effect the peripheral nervous system (Carmeliet et al. Semin. Cell. Dev. Biol. 13: 39-53 (2002)). Delivery of exogenous VEGF can reverse experimental diabetic neuropathy and early evidence from a small clinical trial suggests that this approach could be extended to patients with diabetic neuropathy (Schratzberger et al. J. Clin. Invest. 107: 1083-1092 (2001); Veves et al. J. Clin. Invest. 107: 1215-1218 (2001); Hum. Gene Ther. 12: 1593-1594 (2001)). Strong evidence now indicates that VEGF plays a crucial role in bone development and delivery of exogenous VEGF enhances bone healing (Zelzer et al. Development 129: 1893-1904 (2002); Maes et al. Mech Dev 111: 61-73 (2002); Gerber et al. Nat. Med. 5: 623-628 (1999); Peng et al. J. Clin. Invest. 110: 751-759 (2002); Street et al. Proc. Natl. Acad. Sci. 99: 9656-9661 (2002)). In addition to bone fracture healing, recent evidence also suggests that enhancing VEGF signaling also accelerates healing of skin wounds even in an animal model of diabetes where wound healing is delayed (Di Peppe et al. Gene Ther. 9: 1271-1277 (2002)). Finally, VEGF and VEGF receptors are expressed in hair follicles and transgenic delivery of VEGF in hair follicles enhances hair growth whereas inhibition of VEGF action attenuates hair growth (Yano et al. J. Clin. Invest. 107: 409417 (2001)). Thus enhancing the activation of endothelial PTKs, and VEGF receptors in particular, represents a novel therapeutic approach for heart failure, myocardial infarction, diabetic and ischemic neuropathy (and perhaps other neuropathic conditions), osteoporosis, bone fracture healing, wound healing and hair loss. In view of the foregoing, there is a need to identify inhibitors of PTPase that are useful in a method of treating VEGF-mediated disorders.

Therefore in view of the forgoing, there is a need to identify inhibitors of PTPase that are useful for the treatment of PTPase mediated disorders.

### SUMMARY OF THE INVENTION

The present invention meets the aforementioned need by identifying and providing (1,2,3,4-tetrahydro-isoquinolinyl)-sulfamic acids that are effective in the treating PTPase mediated disorders.

The first aspect of the present invention relates to compounds, including all enantiomeric and diasteriomeric forms and pharmaceutically acceptable salts thereof, having the formula: 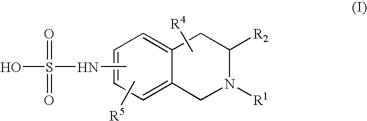
wherein:
A) R¹ is -L¹-[C(R^{6a}R^{6b})]ₘR⁷, wherein:
   a) L¹ is selected from the group consisting of covalent bond, —O—, —S—, —N—, —CO₂—, —CO—, —OCO₂—, —SO—, —SO₂—, —CSN(R⁸)—, —CON(R⁸)O—, —CON(R⁸)—, —OCON(R⁸)—; wherein R⁸ is hydrogen or substituted or unsubstituted C₁-C₅ alkyl;
   b) R^{6a} and R^{6b} are each independently selected from the group consisting of hydrogen, —OR⁹, —N(R⁹)₂, —CO₂R⁹, —CON(R⁹)₂, —NHCOR⁹, —NHCO₂R⁹, ═NR⁹, —R⁹, and mixtures thereof; wherein each R⁹ is independently selected from the group consisting of hydrogen, substituted or unsubstituted C₁-C₅ alkyl, and substituted or unsubstituted aryl or alkylenearyl; or two R⁹ units can be taken together to form a substituted or unsubstituted carbocyclic or heterocyclic ring comprising from 3 to 7 atoms;
   c) m is an index selected from 0 to 5;
   d) R⁷ is selected from the group consisting of nil, hydrogen, substituted or unsubstituted C₁-C₁₀ alkyl, substituted or unsubstituted C₁-C₁₀ heteroalkyl, substituted or unsubstituted hydrocarbyl, substituted or unsubstituted heterocyclyl, substituted or unsubstituted aryl or alkylenearyl, substituted or unsubstituted heteroaryl or alkyleneheteroaryl; or
   e) R⁷ and a R⁹ can be taken together to form a substituted or unsubstituted carbocyclic or heterocyclic ring comprising from 3 to 7 atoms;
B) R² is —(CH₂)ⱼ-L²-[C(R^{11a}R^{11b})]_{g}R¹², wherein:
   a) j is an index selected from 0 to 5;
   b) L is selected from the group consisting of covalent bond, —O—, —S—, —N—, —CO₂—, —CO—, —OCO₂—, —SO—, —SO₂—, —CSN(R¹⁰)—, —CON(R¹⁰)—, —CON(R¹⁰)O—, —OCON(R¹⁰)—; wherein R¹⁰ is hydrogen or substituted or unsubstituted C₁-C₅ alkyl;
   c) R^{11a} and R^{11b} are each independently selected from the group consisting of hydrogen, —OR¹³, —N(R¹³)₂, —CO₂R¹³, —CON(R¹³)₂, —NHCOR¹³, —NHCO₂R¹³, ═NR¹, —R¹³, and mixtures thereof; wherein each R¹³ is independently selected from the group consisting of hydrogen, substituted or unsubstituted C₁-C₅ alkyl, and substituted or unsubstituted aryl or alkylenearyl; or two R¹³ units can be taken together to form a substituted or unsubstituted carbocyclic or heterocyclic ring comprising from 3 to 7 atoms;
   d) g is an index selected from 0 to 5;
   e) R 2 is selected from the group consisting of nil, hydrogen, substituted or unsubstituted C₁-C₁₀ alkyl, substituted or unsubstituted hydrocarbyl, substituted or unsubstituted heterocyclyl, substituted or unsubstituted aryl or alkylenearyl, substituted or unsubstituted heteroaryl or alkyleneheteroaryl; or
   f) R¹² and a R¹³ can be taken together to form a substituted or unsubstituted carbocyclic or heterocyclic ring comprising from 3 to 7 atoms; and
C)R⁴ and R⁵ are each independently selected from hydrogen or substitution unit.

Another aspect of the invention provides a pharmaceutical composition comprising a safe and effective amount of an above-identified compound and a pharmaceutically acceptable carrier.

Another aspect of the invention provides a method of administering to a subject in need thereof a safe and effective amount of an above-identified compound for the treatment of a PTPase mediated disorder.

These and other objects, features, and advantages will become apparent to those of ordinary skill in the art from a reading of the following detailed description and the appended claims.

### DETAILED DESCRIPTION OF THE INVENTION

I. Terms and Definitions

The following is a list of definition for terms used herein:

The term “hydrocarbyl,” as defined herein, means any organic unit or moiety which is comprised of carbon atoms and hydrogen atoms. Included within the term hydrocarbyl are the heterocycles which are described herein below. Examples of various unsubstituted non-heterocyclic hydrocarbyl units include pentyl, 3-ethyloctanyl, 1,3-dimethylphenyl, cyclohexyl, cis-3-hexyl, 7,7-dimethylbicyclo[2.2.1]-heptan-1-yl, and naphth-2-yl.

Included within the definition of “hydrocarbyl” are the aromatic (aryl) and non-aromatic carbocyclic rings, non-limiting examples of which include cyclopropyl, cyclobutanyl, cyclopentanyl, cyclohexane, cyclohexenyl, cycloheptanyl, bicyclo-[0.1.1]-butanyl, bicyclo-[0.1.2]-pentanyl, bicyclo-[0.1.3]-hexanyl (thujanyl), bicyclo-[0.2.2]-hexanyl, bicyclo-[0.1.4]-heptanyl (caranyl), bicyclo-[2.2.1]-heptanyl (norboranyl), bicyclo-[0.2.4]-octanyl (caryophyllenyl), spiropentanyl, diclyclopentanespiranyl, decalinyl, phenyl, benzyl, naphthyl, indenyl, 2H-indenyl, azulenyl, phenanthryl, anthryl, fluorenyl, acenaphthylenyl, 1,2,3,4-tetrahydronaphthalenyl, and the like.

The term “heterocycle” includes both aromatic (heteroaryl) and non-aromatic heterocyclic rings non-limiting examples of which include: pyrrolyl, 2H-pyrrolyl, 3H-pyrrolyl, pyrazolyl, 2H-imidazolyl, 1,2,3-triazolyl, 1,2,4-triazolyl, isoxazolyl, oxazoyl, 1,2,4-oxadiazolyl, 2H-pyranyl, 4H-pyranyl, 2H-pyran-2-one-yl, pyridinyl, pyridazinyl, pyrimidinyl, pyrazinyl, piperazinyl, s-triazinyl, 4H-1,2-oxazinyl, 2H-1,3-oxazinyl, 1,4-oxazinyl, morpholinyl, azepinyl, oxepinyl, 4H-1,2-diazepinyl, indenyl 2H-indenyl, benzofuranyl, isobenzofuranyl, indolyl, 3H-indolyl, 1H-indolyl, benzoxazolyl, 2H-1-benzopyranyl, quinolinyl, isoquinolinyl, quinazolinyl, 2H-1,4-benzoxazinyl, pyrrolidinyl, pyrrolinyl, quinoxalinyl, furanyl, thiophenyl, benzimidazolyl, and the like each of which can be substituted or unsubstituted.

An example of a unit defined by the term “alkylenearyl” is a benzyl unit having the formula: 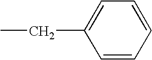
whereas an example of a unit defined by the term “alkyleneheteroaryl” is a 2-picolyl unit having the formula:

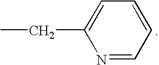

The term “substituted” is used throughout the specification. The term “substituted” is defined herein as “encompassing moieties or units which can replace a hydrogen atom, two hydrogen atoms, or three hydrogen atoms of a hydrocarbyl moiety. Also substituted can include replacement of hydrogen atoms on two adjacent carbons to form a new moiety or unit.” For example, a substituted unit that requires a single hydrogen atom replacement includes halogen, hydroxyl, and the like. A two hydrogen atom replacement includes carbonyl, oximino, and the like. A two hydrogen atom replacement from adjacent carbon atoms includes epoxy, and the like. Three hydrogen replacement includes cyano, and the like. An epoxide unit is an example of a substituted unit which requires replacement of a hydrogen atom on adjacent carbons. The term substituted is used throughout the present specification to indicate that a hydrocarbyl moiety, inter alia, aromatic ring, alkyl chain, can have one or more of the hydrogen atoms replaced by a substituent. When a moiety is described as “substituted” any number of the hydrogen atoms may be replaced. For example, 4-hydroxyphenyl is a “substituted aromatic carbocyclic ring”, (N,N-dimethyl-5-amino)octanyl is a “substituted C₈ alkyl unit, 3-guanidinopropyl is a “substituted C₃ alkyl unit,” and 2-carboxypyridinyl is a “substituted heteroaryl unit.” The following are non-limiting examples of substituted units which can serve as a replacement for hydrogen atoms when a hydrocarbyl unit is described as “substituted.”
i) —[C(R¹⁵)₂]ₚ(CH═CH)_{q}R¹⁵;
ii) —[C(R¹⁵)₂]ₚC(Z)R¹⁵;
iii) —[C(R¹⁵)₂]ₚC(Z)₂R¹⁵;
iv) —[C(R¹⁵)₂]ₚC(Z)CH═CH₂;
v) —[C(R¹⁵)₂]ₚC(Z)N(R¹⁵)₂;
vi) —[C(R¹⁵)₂]ₚC(Z)NR¹⁵N(R¹⁵)₂;
vii) —[C(R¹⁵)₂]ₚCN;
viii) —[C(R¹⁵)₂]ₚCNO;
ix) —[C(R¹⁵)₂]ₚCF₃, —[C(R¹⁵)₂]ₚCCl₃, —[C(R¹⁵)₂]ₚCBr₃;
x) —[C(R¹⁵)₂]ₚN(R¹⁵)₂;
xi) —[C(R¹⁵)₂]ₚNR¹⁵CN;
xii) —[C(R¹⁵)₂]ₚNR¹ C(Z)R¹⁵;
xiii) —[C(R¹⁵)₂]ₚNR¹⁵C(Z)N(R¹⁵)₂;
xiv) —[C(R¹⁵)₂]ₚNHN(R¹⁵)₂;
xv) —[C(R¹⁵)₂]ₚNHOR¹⁵;
xvi) —[C(R¹⁵)₂]ₚNHSO₃M;
xvi) —[C(R¹⁵)₂]ₚNCS;
xvii) —[C(R¹⁵)₂]ₚNO₂;
xviii) —[C(R¹⁵)₂]ₚOR¹⁵;
xix) —[C(R¹⁵)₂]ₚOCN;
xx) —[C(R¹⁵)₂]ₚOCF₃, —[C(R¹⁵)₂]ₚOCCl₃, —[C(R¹⁵)₂]ₚOCBr₃;
xxi) —[C(R¹⁵)₂]ₚF, —[C(R¹⁵)₂]ₚCl, —[C(R¹⁵)₂]ₚBr, —[C(R¹⁵)₂]ₚI, and mixtures thereof;
xxii) —[C(R¹⁵)₂]ₚSCN;
xxiii) —[C(R¹⁵)₂]ₚSO₃M;
xxiv) —[C(R¹⁵)₂]ₚOSO₃M;
xxv) —[C(R¹⁵)₂]ₚSO₂N(R¹⁵)₂;
xxvi) —[C(R¹⁵)₂]ₚSO₂NH(R¹⁵);
xxvii) —[C(R⁵)₂]ₚSO₂NHCOR¹⁵;
xxviii) —[C(R¹⁵)₂]ₚSO₂NHCOOR¹⁵;
xxvi) —[C(R⁵)₂]ₚSO₂R¹⁵;
xxvii) —[C(R¹⁵)₂]ₚP(O)H₂;
xxviii) —[C(R¹⁵)₂]ₚPO₂;
xxix) —[C(R¹⁵)₂]ₚP(O)(OH)₂;
xxix) —[C(R¹⁵)₂]ₚCO₂M;
xxx) —[C(R¹⁵)₂]ₚSR¹⁵;
xxxi) and mixtures thereof;
   wherein R¹⁵ is hydrogen, substituted or unsubstituted C₁-C₂₀ linear, branched, or cyclic alkyl, C₆-C₂₀ aryl, C₇-C₂₀ alkylenearyl, and mixtures thereof; M is hydrogen, or a salt forming cation; Z is ═O, ═S, ═NR¹⁵, and mixtures thereof; p is from 0 to 12; q is from 0 to 12. Suitable salt forming cations include, sodium, lithium, potassium, calcium, magnesium, ammonium, and the like.
   II. Compounds

A first aspect of the present invention relates to compounds having the formula: 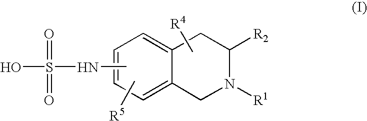
wherein R¹, R², R⁴, R⁵ are previously defined.

A second aspect of the present invention relates to compounds having the formula (II): 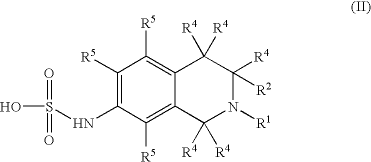
wherein R¹, R², R⁴, R⁵ are previously defined.

The following are the various aspects of non-limiting preferred moieties; however, the formulator is not limited to the herein exemplified iterations and examples.
A) The single sulfamic acid moiety (HOSO₂NH—) of formula (I) is attached at the 5, 6, 7, or 8-position of the 1,2,3,4-tetrahydroisoquinoline scaffold. Conventional numbering is herein presented: 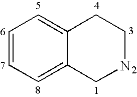
In one embodiment, the sulfamic acid moiety is at the 6 or 7-position of the scaffold. In another embodiment, the sulfamic acid moiety is at the 7-position of the scaffold.

B) R¹ is -L¹-[C(R^{6a}R^{6b})]ₘR⁷. L¹ is selected from the group consisting of covalent bond, —O—, —S—, —N—, —CO₂—, —CO—, —OCO₂—, —SO—, —SO₂—, —CSN(R⁸)—, —CON(R⁸)O—, —CON(R⁸)—, —OCON(R¹)—. R⁸ is hydrogen or substituted or unsubstituted C₁-C₅ alkyl. R^{6a} and R^{6b} are each independently selected from the group consisting of hydrogen, —OR⁹, —N(R⁹)₂, —CO₂R⁹, —CON(R⁹)₂, —NHCOR⁹, —NHCO₂R⁹, ═NR⁹, —R⁹, and mixtures thereof. In turn, each R⁹ is independently selected from the group consisting of hydrogen, substituted or unsubstituted C₁-C₅ alkyl, and substituted or unsubstituted aryl or alkylenearyl; or two R⁹ units can be taken together to form a substituted or unsubstituted carbocyclic or heterocyclic ring comprising from 3 to 7 atoms. m is an index selected from 0 to 5. R⁷ is selected from the group consisting of nil hydrogen, substituted or unsubstituted C₁-C₁₀ alkyl, substituted or unsubstituted C₁-C₁₀ heteroalkyl, substituted or unsubstituted hydrocarbyl, substituted or unsubstituted heterocyclyl, substituted or unsubstituted aryl or alkylenearyl, substituted or unsubstituted heteroaryl or alkyleneheteroaryl. Alternatively,
R⁷ and a R⁹ can be taken together to form a substituted or unsubstituted carbocyclic or heterocyclic ring comprising from 3 to 7 atoms.

In one embodiment, L¹ is selected from the group consisting of covalent bond, —CO₂—, —SO₂—, and —CON(R⁸)—. In another embodiment, R⁸ is hydrogen. In another embodiment, L¹ is selected from covalent bond or —CO—.

In one embodiment, R^{6a} and R^{6b} are each hydrogen.

In one embodiment, m is an index selected from 0 to 5. In another embodiment, R^{6a} and R^{6b} are each hydrogen and m is an index selected from 1-5.

In one embodiment, R⁷ is substituted or unsubstituted C₁-C₁₀ alkyl. In another embodiment, R⁷ is C₁-C₁₀ alkyl wherein the alkyl is branched. In another embodiment, R⁷ is selected from the group consisting of —CH₃, —CH₂CH₃, —CH₂CH₂CH₃, —(CH2)₃, —(CH₂)₄CH₃, —(CH₂)₅CH₃, —C(CH₃)₅, —CH₂C(CH₃)₃, —CH₂C(CH₃)₂CH₂CH₃, —C(CH₃)₂CH₂CH₃, —CH(CH₃)CH₂CH₃, and —CH₂CH(CH₂CH₃)₂. In another embodiment, R⁷ is substituted C₁-C₁₀ alkyl and the substituted unit is —COOH. In another embodiment, R⁷ is selected from the group consisting of —CH₂COOH, —CH₂CH₂COOH, and —CH₂CH₂CH₂COOH.

In one embodiment, R⁷ is substituted or unsubstituted alkylenearyl. In another embodiment, R⁷ is substituted or unsubstituted C₇-C₁₂ alkylenearyl is —CH₂(C₆H₅), —CH₂CH₂(C₆H₅), —(CH₂)₃C₆H₅, —(CH₂)₄C₆H₅, —CH₂(C₁₀H₇), —CH₂CH₂(C₁₀H₇), —(CH₂)₃C₁₀H₇, and —(CH₂)₄C₁₀H₇. In another embodiment, R^{7 is} alkylenearyl substituted with at least a substituted unit selected from group consisting of methyl, ethyl, propyl, butyl, methoxy, ethoxy, propyloxy, butoxy, trifluoromethyl, sulfamic acid, hydroxy, and mixtures thereof. In another embodiment, R⁷ is alkylenearyl is substituted with at least a substituted unit selected from the group consisting of —[C(R⁵)₂]ₚSO₂N(R¹⁵)₂, —[C(R¹⁵)₂]ₚSO₂NH(R¹⁵), —[C(R⁵)₂]ₚSO₂NHCOR¹⁵; and —[C(R¹⁵)₂]ₚSO₂NHCOR¹⁵; wherein the substituted unit is still in another embodiment selected from the group consisting of —SO₂NH₂, —SO₂NHCOOCH₃, —SO₂NHCOOCH₂CH₃, —SO₂NHCOCH₃, —SO₂NHCOCH₂CH₃, —SO₂NHCOC(CH₃)₃, SO₂NH(C₆H₅), —SO₂NHCO(C₆H₅), —SO₂NHCOCH₂(C₆H₅), and —SO₂NHCOCH₂CH₂(C₆H₅).

In one embodiment, R⁷ is substituted or unsubstituted alkyleneheteroaryl. In one embodiment, the R⁷ substituted alkeneheteroaryl is substituted with at least a substituted unit selected from the group consisting of methyl, ethyl, propyl, butyl, methoxy, ethoxy, propyloxy, butoxy, trifluoromethyl, sulfamic acid, hydroxy, and mixtures thereof.

B) R² is —(CH₂)ⱼ-L²-[C(R^{11a}R^{11b})]_{g}R¹². Index j is selected from 0 to 5. L² is selected from the group consisting of covalent bond, —O—, —S—, —N—, —CO₂—, —CO—, —OCO₂—, —SO—, —SO₂—, —CSN(R¹⁰)—, —CON(R¹⁰)—, —CON(R¹⁰)O—, —OCON(R¹⁰)—. In turn, R¹⁰ is selected from hydrogen or substituted or unsubstituted C₁-C₅ alkyl. R^{11a} and R^{11b} are each independently selected from the group consisting of hydrogen, —OR¹³, —N(R¹³)₂, —CO₂R¹³, —CON(R³)₂, —NHCOR¹³, —NHCO₂R³, ═NR¹³, —R¹³, and mixtures thereof. Each R¹³ is independently selected from the group consisting of hydrogen, substituted or unsubstituted C₁-C₅ alkyl, and substituted or unsubstituted aryl or alkylenearyl; or two R¹³ units can be taken together to form a substituted or unsubstituted carbocyclic or heterocyclic ring comprising from 3 to 7 atoms. Index g is selected from 0 to 5. R¹² is selected from the group consisting of nil, hydrogen, substituted or unsubstituted C₁-C₁₀ alkyl, substituted or unsubstituted hydrocarbyl, substituted or unsubstituted heterocyclyl, substituted or unsubstituted aryl or alkylenearyl, substituted or unsubstituted heteroaryl or alkyleneheteroaryl. Alternatively, R¹² and a R¹³ can be taken together to form a substituted or unsubstituted carbocyclic or heterocyclic ring comprising from 3 to 7 atoms.

In one embodiment, index j is 0. In another embodiment, index j is 1.

In one embodiment, index m is 0.

In one embodiment, L² is selected from the group consisting of covalent bond, —CONR₈—CONH—, —CON(CH₃)—, —CO₂—, —CO—, and mixtures thereof. In another embodiment, L² is selected from the group consisting of CONR₈—, —CONH—, and mixtures thereof.

In one embodiment, R¹² is substituted and unsubstituted alkyl. In one embodiment, R¹² is selected from —CH₃, —CH₂CH₃, —CH₂CH₂CH₃, —(CH₂)₃, —(CH₂)₄CH₃, —(CH₂)₅CH₃, —C(CH₃)₅, —CH₂C(CH₃)₃, —CH₂C(CH₃)₂CH₂CH₃, —C(CH₃)₂CH₂CH₃, —CH(CH₃)CH₂CH₃, and —CH₂CH(CH₂CH₃)₂. In one embodiment R¹² is substituted alkyl, wherein the substituted unit is at least one or more from the group consisting of —OH and —COOH.

In one embodiment, R¹² is from substituted or unsubstituted C₇-C₁₀ alkylenearyl. In another embodiment, R12 is —CH₂(C₆H₅), —CH₂CH₂(C₆H₅), —(CH₂)₃(C₆H₅), —(CH₂)₄(C₆H₅), —CH₂(C₁₀H₇), —CH₂CH₂(C₁₀H₇), —(CH₂)₃(C₁₀H₇), and —(CH₂)₄(CO₁₀H₇).

In one embodiment, R² is hydrogen.

C)R⁴ and R⁵ are each independently selected from hydrogen or substituted unit. In one embodiment, R⁴ and R⁵ are each hydrogen.

III. Compound Preparation

The compounds of the invention can be prepared using a variety of procedures. The starting materials used in preparing the compounds of the invention are known, made by known methods, or are commercially available. Particularly preferred syntheses are described in the following general reaction schemes. (The R groups used to illustrate the reaction schemes do not necessarily correlate to the respective R groups used to describe the various aspects of the Formula (I) compounds. That is, for example, R₁ in Formula (I) does not represent the same moiety as R₁ here.) Specific examples for making the compounds of the present invention are set forth in Section VI, below.

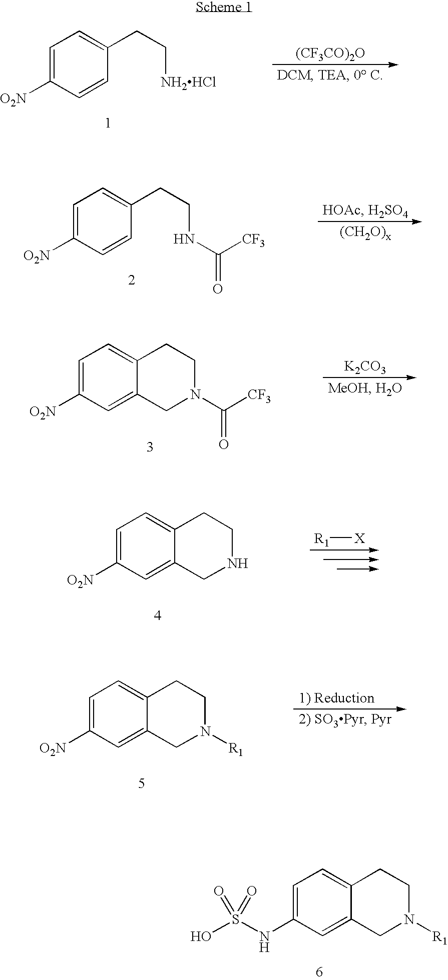

In reference to Scheme 1, the synthesis of intermediate 7-nitro-1,2,3,4-tetrahydro-isoquinoline (4) follows from methods well-known in the art. See, e.g., Stokker, Tetrahedron Lett. (1996), 37, 5453-5456; and Xie, et al., Synth. Commun. (2000) 30, 1581-1585. Briefly, 4-nitro-phenethyl amine hydrochloride (1) is dissolved in a non-protic solvent such as dichloromethane with the aid of an appropriate tertiary amine base such as triethyl amine. The resulting homogeneous solution is cooled to at or near 0° C. and treated with trifluoroacetic anhydride. The reaction is allowed to warm to room temperature (1 h) at which point analysis by thin layer chromatography shows the reaction to be complete. Aqueous work-up provides the trifluoroacetamide (2) in high yield. It is understood that there are additional methods employing different solvents, bases, triflouoracetylating reagents, time and temperature that can be employed in this transformation many of which can be found, inter alia, in the text by Greene and Wuts (Chapter 7. Protective Groups in Organic Synthesis, 3^{rd} ed.; Wiley & Sons: New York, 1999, 556-558). The triflouroacetamide (2) and paraformaldehyde are added to a solution of acetic acid in sulfuric acid (ratio 2:3, V/V) at room temperature and the resulting solution is stirred for a period of at least 16 h and following aqueous work-up the desired cyclized material (3) is isolated in high yield and purity. Deprotection of the triflouroacetamide (3) is effected by the use of aqueous potassium carbonate in methanol over a period about 30 min. to provide the 7-nitro-1,2,3,4-tetrahydro-isoquinoline (4) cleanly. Additional methods for this transformation can also be found in the previously cited text by Greene and Wuts (vide supra).

Intermediate (4) is the substrate for a number of subsequent functionalization reactions whereby numerous electrophilic reagents (R—X) are introduced under the appropriate conditions. Non-limiting examples of these types of reactions include urea formation by the addition of isocyanates, thio-urea formation by the addition of isothiocyanates, amide formation by the reaction with acid chlorides, amide formation through condensation with carboxylic acids employing an appropriate condensation reagent (See Bodansky, M; Activation and Coupling. In Principles of Peptide Synthesis, 2^{nd} ed.; Springer Publishing: New York, 1993, pp 9-61), carbamate formation by the reaction of chloroformates (see—Greene and Wuts, vide supra), tertiary amino compounds through reductive alkylation with suitable carbonyl compounds, tertiary amine formation through alkylation with alkyl halides, guanidine formation through various methods (see-Burgess, K; Chen, J.; Solid-Phase Synthesis of Guanidines. In Solid Phase Organic Synthesis; Kevin Burgess, Ed.; John Wiley & Sons: New York, 2000; pp 1-23: and references therein) and sulfonamide formation through reaction with sulfonyl chlorides. The above-mentioned functionalization reactions are not to be considered exhaustive but merely illustrative of the types of rountine chemistry, which can be carried out by those skilled in the art of organic synthesis to provide compounds of general structure (5).

Lastly, intermediate (5) is then carried on directly to a final aryl sulfamic acid compound (6) through a two-step process, which involves aryl nitro group reduction (methods for which are numerous, see, e.g., Hudlicky, M; Reduction of Nitro, Nitroso, Diazo, and Azido Derivatives of Hydrocarbons and Basic Heterocycles, In Reductions in Organic Chemistry, 2^{nd} ed.; ACS Monograph 188; American Chemical Society: Washington, D.C., 1996) followed by sulfamic acid formation. Formation of the final sulfamic acid compounds is carried out by dissolution of the reduction product in anhydrous pyridine (ca. 2-3 mL per 0.5 mmol) and addition of solid sulfur trioxide pyridine complex (3 molar eqs.). Upon addition of the sulfur trioxide pyridine complex the reaction mixture is stirred for about 5 min then the reaction is quenched with diluted ammonium hydroxide solution (ca. 7% aqueous). Evaporation of all volatiles provides the crude material, which is generally purified by RP-HPLC to provide the target compounds in typical yields of 30-65% (2-steps). Additionaly, alternative complexes of sulfur trioxide could be employed (i.e. sulfur trioxide dioxane, etc.), with non-limiting examples discussed in Gilbert (Chem. Rev. (1962) 62, 549-89). The reduced nitro compound can also be functionalized as the sulfamic acid by the action of chlorosulfonic acid in the presence of an appropriate base (see, e.g., Sureau, R. F. M, et. al; Preparation of Sulphamic Acids., U.S. Pat. No. 2,789,132) and also by the action of O-Trimethylsilyl chlorosulfonic acid and an appropriate base.

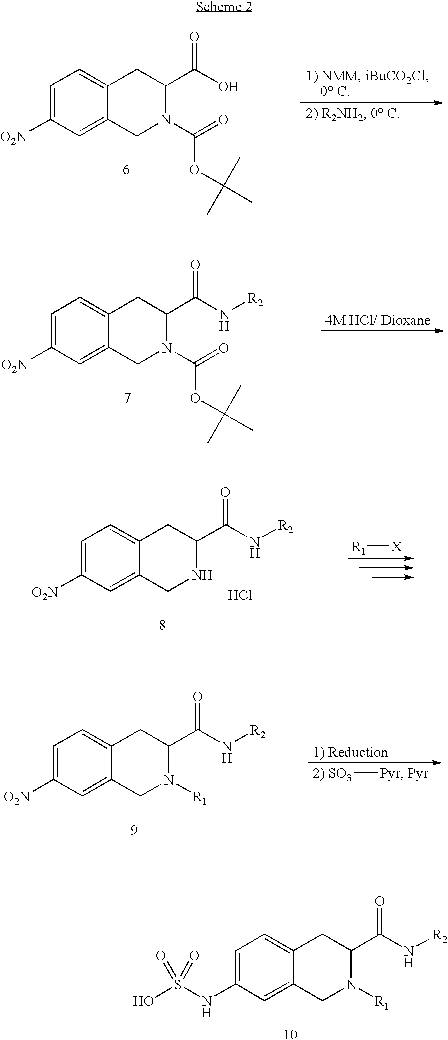

In reference to Scheme 2, starting compound t-butoxycarbonyl (Boc) protected 7-nitro-1,2,3,4-tetrahydro-isoquinoline derivative (6) is subjected to methyl amide formation to yield compound (7) through the formation of the unsymmetrical isobutyl carbonic anhydride and displacement with methylamine. Alternatively, methods presented above Scheme 1 involving the use of condensation reagents for the formation of amides may also be employed. Additionally, numerous amine components could be introduced at this step to add variability to the analog synthesis. Removal of the Boc protecting group is carried out under acidic conditions (see, e.g., Greene and Wuts, pp 518-525) such as using 4 M hydrogen chloride in 1,4-dioxane to provide the hydrochloride (8) in high yield and purity.

Intermediate (8) is a substrate for a number of subsequent functionalization reactions whereby electrophilic reagents (R—X) are introduced under the appropriate conditions to provide intermediates of general structure (9). Lastly, as in Scheme 1, intermediate 9 is then carried on directly to yield the final aryl sulfamic acid compounds of formula (10) through a two-step process, which involves aryl nitro group reduction followed by sulfamic acid formation; and optionally functionalizing thereafter.

A variety of compounds can be generated in a similar fashion, using the guidance of the schemes above.

These steps may be varied to increase yield of desired product. The skilled artisan will recognize the judicious choice of reactants, solvents, and temperatures is an important component in any successful synthesis. Determination of optimal conditions, etc. is routine. Thus the skilled artisan can make a variety of compounds using the guidance of the schemes above.

It is recognized that the skilled artisan in the art of organic chemistry can readily carry out standard manipulations of organic compounds without further direction; that is, it is well within the scope and practice of the skilled artisan to carry out such manipulations. These include, but are not limited to, reduction of carbonyl compounds to their corresponding alcohols, oxidations of hydroxyls and the like, acylations, aromatic substitutions, both electrophilic and nucleophilic, etherifications, esterification and saponification and the like. Examples of these manipulations are discussed in standard texts such as March, Advanced Organic Chemistry (Wiley), Carey and Sundberg, Advanced Organic Chemistry (2 Volumes) and other art that the skilled artisan is aware of.

The skilled artisan will also readily appreciate that certain reactions are best carried out when another potentially reactive functionality on the molecule is masked or protected, thus avoiding any undesirable side reactions and/or increasing the yield of the reaction. Often the skilled artisan utilizes protecting groups to accomplish such increased yields or to avoid the undesired reactions. These reactions are found in the literature and are also well within the scope of the skilled artisan. Examples of many of these manipulations can be found for example in T. Greene, Protecting Groups in Organic Synthesis. Of course, amino acids used as starting materials with reactive side chains are preferably blocked to prevent undesired side reactions.

The compounds of the invention may have one or more chiral centers. As a result, one may selectively prepare one optical isomer, including diastereomer and enantiomer, over another, for example by chiral starting materials, catalysts or solvents, or may prepare both stereoisomers or both optical isomers, including diastereomers and enantiomers at once (a racemic mixture). Since the compounds of the invention may exist as racemic mixtures, mixtures of optical isomers, including diastereomers and enantiomers, or stereoisomers may be separated using known methods, such as chiral salts, chiral chromatography and the like.

In addition, it is recognized that one optical isomer, including diastereomer and enantiomer, or stereoisomer may have favorable properties over the other. Thus when disclosing and claiming the invention, when one racemic mixture is disclosed, it is clearly contemplated that both optical isomers, including diastereomers and enantiomers, or stereoisomers substantially free of the other are disclosed and claimed as well.

IV. Methods of Treating PTPase Mediated Disorders.

The above-identified compounds of the present invention may be used in a method for the treatment of a PTPase mediated disorder. As used herein, a “PTPase mediated disorder” is one that involves unwanted or elevated PTPase activity in the biological manifestation of the disease, disorder, and/or condition; in the biological cascade leading to the disorder; or as a symptom of the disorder. This “involvement” of PTPase in a PTPase mediated disorder includes, but is not limited to, the following: (1) The unwanted or elevated PTPase activity as a “cause” of the disorder or biological manifestation, whether the PTPase is elevated genetically, by infection, by autoimmunity, trauma, biomechanical causes, lifestyle, or by some other causes. (2) The unwanted or elevated PTPase activity is part of the observable manifestation of the disease or disorder. That is, the disease or disorder is measurable in terms of the increased PTPase activity. From a clinical standpoint, unwanted or elevated PTPase activity indicate the disease, however, PTPase activity need not be the “hallmark” of the disease or disorder. (3) The unwanted or elevated PTPase activity is part of the biochemical or cellular cascade that results in the disease or disorder. In this respect, inhibition of PTPase interrupts the cascade, and thus controls the disease. Non-limiting examples of PTPase mediated disorders that may be treated by the present invention include insulin receptor tyrosine mediated disorder and bone disorder.

As used herein, “PTPase” means enzymes with the capacity to dephosphorylate pTyr-containing proteins or glycoproteins or motifs generally. Non-limiting examples of PTPases include: intracellular PTPases (e.g., PTP1B, TC-PTP, PTP1C, PTPID, PTPD1, PTPD2); receptor-type PTPases (e.g., PTPα, PTPε, PTPβ, PTPγ, CD45, PTPκ, PTPμ); dual specificity phosphatases (VH1, VHR, cdc25), LMW-PTPases; and acid phosphatases.

All known intracellular type PTPases contain a single conserved catalytic phosphatase domain consisting of 220-240 amino residues. Non-limiting examples of intracellular type PTPases include: PTPI B (Tonks et al., J. Biol. Chem. 263: 6722-6730 (1988)); PTP1 (Charbonneau et al., Proc. Natl. Acad. Sci. USA 86: 5252-5256 (1989); Chemoff et al., Proc. Natl. Acad. Sci. USA 87: 2735-2789 (1989)); T-cell PTPase (Cool et al. Proc. Natl. Acad. Sci. USA 86: 5257-5261 (1989)); rat brain PTPase (Guan et al., Proc. Natl. Acad. Sci. USA 87: 1501-1502(1990)); neuronal phosphatase STEP (Lombroso et al., Proc. Natl. Acad. Sci. USA 88: 7242-7246 (1991)); ezrin-domain containing PTPases; PTPMEG1 (Gu et al., Proc. Natl. Acad. Sci. USA 88: 5867-57871 (1991)), PTPH I Yang and Tonks, Proc. Natl. Acad. Sci. USA 88: 5949-5953 (1991), PTPD1 and PTPD2 (Moller et al., Proc. Natl. Acad. Sci. USA 91: 7477-7481 (1994)); FAP-1/BAS (Sato et al., Science 268: 411-415 (1995); Banville et al., J. Biol. Chem. 269: 22320-22327 (1994); Maekawa et al., FEBS Letters 337: 200-206 (1994)); and SH2 domain containing PTPases: PTP1C/SH-PTPI (Plutzky et al., Proc. Natl. Acad. Sci. USA 89: 1123-1127 (1992); Shen et al., Nature Lond. 352: 736-739 (1991)) and PTP1D/Syp/SH-PTP2 (Vogel et al., Science 259: 1611-1614 (1993); Feng et al., Science 259: 1607-1611 (1993); Bastein et al., Biochem, Biophys. Res. Comm. 196: 124-133 (1993)).

Most receptor-type PTPases consist of a) a putative ligand-binding extracellular domain, b) a transmembrane segment, and c) an intracellular catalytic region. Non-limiting examples include CD45/LCA (Ralph, S. J., EMBO J. 6: 1251-1257 (1987)); LAR (Streuli et al., J. Exp. Med. 168: 1523-1530 (1988); Charbonneau et al., Proc. Natl. Acad. Sci. USA 86: 5252-5256 (1989)); CD45 (Trowbridge and Thomas, Ann. Rev. Immunol. 12: 85-116 (1994)); PTPα Krueger et al., EMBO J. 9: 3241-3252 (1990)); PTPβ (Krueger supra); PTPδ (Krueger supra); PTPε (Krueger supra); PTPζ (Krueger supra). Other examples of receptor type PTPases include PTPγ (Bamea et al., Mol. Cell. Biol. 13: 1497-1506 (1995); PTPμ (Gebbink et al., FEBS Letters 290: 123-130 (1991); PTPK (Jiang et al., Mol. Cell. Biol. 13: 2942-2951 (1993); SAP-1 (Matozaki et al., J. Biol. Chem. 269: 2075-2081(1994)); and PTP-U2/GLEPP1 (Seimiya et al., Oncogene 10: 1731-1738 (1995); (Thomas et al., J. Biol. Chem. 269: 19953-19962 (1994)). Novel PTPases are continuously identified, and it is anticipated that more than 500 different species will be found in the human genome, i.e., close to the predicted size of the protein tyrosine kinase superfamily (Hanks and Hunter, FASEB J. 9: 576-596 (1995)).

Dual specificity protein tyrosine phosphatases (dsPTPases) define a subclass within the PTPases family that can hydrolyze phosphate from phosphotyrosine as well as from phospho-serine/threonine. dsPTPases contain the signature sequence of PTPases: His-Cys-Xxx-Xxx-Gly-Xxx-Xxx-Arg. At least three dsPTPases have been shown to dephosphorylate and inactivate extracellular signal-regulated kinase (ERKs)/mitogen-activated protein kinase (MAPK): MAPK phosphatase (CL100, 3CH134) (Charles et al., Proc. Natl. Acad. Sci. USA 90: 5292-5296 (1993)); PAC-1 (Ward et al., Nature 367: 651-654 (1994)); rVH6 (Mourey et al., J. Biol. Chem. 271: 3795-3802 (1996)). Transcripton of dsPTPases are induced by different stimuli, e.g., oxidative stress or heat shock (Ishibashi et al., J. Biol. Chem. 269: 29897-29902 (1994); Keyse and Emslie, Nature 359: 644-647 (1992)). Further, they may be involved in regulation of the cell cycle: cdc25 (Millar and Russell, Cell 68: 407-410 (1992)); KAP (Hannon et al. Proc. Natl. Acad. Sci. USA 91: 1731-1735 (1994); review by Walton and Dixon, Annu. Rev. Biochem. 62: 101-120 (1993)).

Low molecular weight phosphotyrosine-protein phosphatase (LMW-PTPase) shows little sequence identity to the intracellular PTPases described above. However, this enzyme belongs to the PTPase family due to at least possessing the PTPase active site motif (Cirri et al., Eur. J. Biochem. 214: 647.657 (1993). For further rationales, see Chiarugi et al., FEBS Lett. 310: 9-12 (1992) and Su et al., Nature 370: 575-578 (1994).

To determine and assess the PTPase inhibition activity testing of the subject compounds is carried using various assays known to those skilled in the art. For example, a DiFMUP Phosphatase Assay is described. DIFMUP (“6,8-difluoro-4-methylumbelliferyl phosphate”) (Molecular Probes) (10 MM) is incubated for 15 minutes with nM concentrations of phosphatase in buffer containing 50 mM Tris (pH 7), 150 mM NaCl, 5 mM DTT, 1 mM EDTA, 0.01% BSA. The resulting phosphatase product is measured at 355/460 nm (ex/em) using a Victor V plate reader (Wallac). Inhibitors (0.002-40 mM) are pre-incubated with phosphatase for 10 minutes prior to addition of DiFMUP substrate. IC₅₀ curves are generated using Excel-Fit®.

C. Methods of Treatment

The compounds of the present invention may be useful in a method of treating a PTPase mediated disorder in a subject in need of such treatment comprising administering of a compound of the present invention.

The term “treatment” is used herein to mean that, at a minimum, administration of a compound of the present invention mitigates a disease associated with a PTPase mediated disorder in a subject, preferably in a mammalian subject, more preferably in humans. Thus, the term “treatment” includes: preventing an PTPase mediated disorder in a subject, particularly when the subject is predisposed to acquiring the disease, but has not yet been diagnosed with the disease; inhibiting the PTPase mediated disorder; and/or alleviating or reversing the PTPase mediated disorder. Insofar as the methods of the present invention are directed to preventing PTPase mediated disorder, it is understood that the term “prevent” does not require that the disease state be completely thwarted. (See Webster's Ninth Collegiate Dictionary.) Rather, as used herein, the term preventing refers to the ability of the skilled artisan to identify a population that is susceptible to PTPase mediated disorder, such that administration of the compounds of the present invention may occur prior to onset of PTPase mediated disorder. The term does not imply that the disease state be completely avoided. The population that is at risk of a PTPase mediated disorder, for example as diabetes type I, are those who have a genetic predisposition to diabetes as indicated by family history of the disease. Other risk factors include obesity or diet.

Different embodiments of PTPase mediated disorders of the present invention herein follow.

1. Insulin Receptor Mediated Disorder.

In one aspect of the invention, the PTPase mediated disorder is an insulin receptor tyrosine kinase mediated disorder. As used herein, “insulin receptor tyrosine mediated disorder” is a disease or disorder that involves defects in insulin receptor tyrosine signaling thereby resulting in the biological manifestation of the disorder; in the biological cascade leading to the disorder; or as a symptom of the disorder. In one embodiment, the insulin receptor tyrosine kinase mediated disorder is selected from the group consisting of type I diabetes, type II diabetes, impaired glucose tolerance, insulin resistance and obesity. In another embodiment, the disorder is type II diabetes.

In order to determine and assess the pharmacological activity against an insulin receptor tyrosine kinase mediated disorder, testing of the subject compounds in animals is carried using various assays known to those skilled in the art. For example, the activity of the subject compounds against diabetes can be measured using an assay designed to measure blood sugar levels in mice with diabetes experimentally induced by alloxan.

2. Bone Disorders.

In one aspect of the invention, the PTPase mediated disorder is a bone disorder. As used herein, “bone disorder” is a disease or disorder that involves defects in osteoblast proliferation thereby resulting in the biological manifestation of the disorder; in the biological cascade leading to the disorder; or as a symptom of the disorder. In one embodiment, the bone disorder is selected from the group consisting of osteoporosis and Paget's disease.

In order to determine and assess the pharmacological activity against a bone disorder, testing of the subject compounds in animals is carried out using various assays known to those skilled in the art. For example, the activity of the subject compounds against a bone disorder can be conveniently demonstrated using an assay designed to test the ability of the subject compounds to increase bone volume, mass, or density. An example of such an assay is the ovariectomized rat assay. In the overiectomized rat assay, six-month old rats are ovariecotmized, aged 2 months, and the dosed once a day subcutaneously with a test compound. Upon completion of the study, bone mass and/or density can be measured by dual energy X-ray absorptometry (DXA) or peripheral quantitative computed tomography (pQCT), or micro computed tomography (mCT). Alternatively, static and dynamic histomorphometry can be used to measure the increase in bone volume or formation.

3. Angiogenesis-Mediated Disorders

In one aspect of the invention, the PTPase mediated disorder is an angiogenesis mediated disorder. As used herein, “angiogenesis” means the formation of new blood vessels from pre-existing vasculature. As used herein, “angiogenesis mediated disorders” include: (1) those disorders, diseases and/or unwanted conditions which are characterized by unwanted or elevated angiogenesis referred to herein collectively as “angiogenesis elevated disorders;” or (2) those disorders, diseases and/or unwanted conditions which are characterized by wanted or reduced angiogenesis referred to herein collectively as “angiogenesis reduced disorders.”

a. Angiogenesis Elevated Disorder

As used herein, an “angiogenesis elevated disorder” is one that involves unwanted or elevated angiogenesis in the biological manifestation of the disease, disorder, and/or condition; in the biological cascade leading to the disorder; or as a symptom of the disorder. This “involvement” of angiogenesis in an angiogenesis elevated disorder includes, but is not limited to, the following: (1) The unwanted or elevated angiogenesis as a “cause” of the disorder or biological manifestation, whether the level of angiogenesis is elevated genetically, by infection, by autoimmunity, trauma, biomechanical causes, lifestyle, or by some other causes. (2) The angiogenesis as part of the observable manifestation of the disease or disorder. That is, the disease or disorder is measurable in terms of the increased angiogenesis. From a clinical standpoint, unwanted or elevated angiogenesis indicate the disease, however, angiogenesis need not be the “hallmark” of the disease or disorder. (3) The unwanted or elevated angiogenesis is part of the biochemical or cellular cascade that results to the disease or disorder. In this respect, inhibition of angiogenesis interrupts the cascade, and thus controls the disease. Non-limiting examples of angiogenesis reduced disorders that may be treated by the present invention are herein described below.

The compounds of the present invention may be used to treat diseases associated with retinal/choroidal neovascularization that include, but are not limited to, diabetic retinopathy, macular degeneration, sickle cell anemia, sarcoid, syphilis, pseudoxanthoma elasticum, Paget's disease, vein occlusion, artery occlusion, carotid obstructive disease, chronic uveitis/vitritis, mycobacterial infections, Lyme's disease, systemic lupus erythematosis, retinopathy of prematurity, Eales' disease, Behcet's disease, infections causing a retinitis or choroiditis, presumed ocular histoplasmosis, Best's disease, myopia, optic pits, Stargardt's disease, pars planitis, chronic retinal detachment, hyperviscosity syndromes, toxoplasmosis, trauma and post-laser complications. Other diseases include, but are not limited to, diseases associated with rubeosis (neovasculariation of the angle) and diseases caused by the abnormal proliferation of fibrovascular or fibrous tissue including all forms of proliferative vitreoretinopathy, whether or not associated with diabetes.

Compounds of the present invention can treat diseases associated with chronic inflammation. Diseases with symptoms of chronic inflammation include inflammatory bowel diseases such as Crohn's disease and ulcerative colitis, psoriasis, sarcoidosis and rheumatoid arthritis. Angiogenesis is a key element that these chronic inflammatory diseases have in common. The chronic inflammation depends on continuous formation of capillary sprouts to maintain an influx of inflammatory cells. The influx and presence of the inflammatory cells produce granulomas and thus, maintains the chronic inflammatory state. Inhibition of angiogenesis by the compositions and methods of the present invention would prevent the formation of the granulomas and alleviate the disease.

Compounds may be used to treat patients with inflammatory bowel diseases such as Crohn's disease and ulcerative colitis. Both Crohn's disease and ulcerative colitis are characterized by chronic inflammation and angiogenesis at various sites in the gastrointestinal tract. Crohn's disease is characterized by chronic granulomatous inflammation throughout the gastrointestinal tract consisting of new capillary sprouts surrounded by a cylinder of inflammatory cells. Prevention of angiogenesis by the compounds of the present invention inhibits the formation of the sprouts and prevents the formation of granulomas. Crohn's disease occurs as a chronic transmural inflammatory disease that most commonly affects the distal ileum and colon but may also occur in any part of the gastrointestinal tract from the mouth to the anus and perianal area. Patients with Crohn's disease generally have chronic diarrhea associated with abdominal pain, fever, anorexia, weight loss and abdominal swelling. Ulcerative colitis is also a chronic, nonspecific, inflammatory and ulcerative disease arising in the colonic mucosa and is characterized by the presence of bloody diarrhea.

The inflammatory bowel diseases also show extraintestinal manifestations such as skin lesions. Such lesions are characterized by inflammation and angiogenesis and can occur at many sites other than the gastrointestinal tract. The compounds of the present invention may be capable of treating these lesions by preventing the angiogenesis, thus reducing the influx of inflammatory cells and the lesion formation.

Sarcoidosis is another chronic inflammatory disease that is characterized as a multisystem granulomatous disorder. The granulomas of this disease may form anywhere in the body and thus the symptoms depend on the site of the granulomas and whether the disease active. The granulomas are created by the angiogenic capillary sprouts providing a constant supply of inflammatory cells.

Compounds of the present invention can also treat the chronic inflammatory conditions associated with psoriasis. Psoriasis, a skin disease, is another chronic and recurrent disease that is characterized by papules and plaques of various sizes. Prevention of the formation of the new blood vessels necessary to maintain the characteristic lesions leads to relief from the symptoms.

Another disease that may be treated according to the present invention, is rheumatoid arthritis. Rheumatoid arthritis is a chronic inflammatory disease characterized by nonspecific inflammation of the peripheral joints. It is believed that the blood vessels in the synovial lining of the joints undergo angiogenesis. In addition to forming new vascular networks, the endothelial cells release factors and reactive oxygen species that lead to pannus growth and cartilage destruction. The factors involved in angiogenesis may actively contribute to, and help maintain, the chronically inflamed state of rheumatoid arthritis. Other diseases that can be treated according to the present invention are hemangiomas, Osler-Weber-Rendu disease, or hereditary hemorrhagic telangiectasia, solid or blood borne tumors and acquired immune deficiency syndrome.

b. Angiogenesis Reduced Disorder

As used herein, an “angiogenesis reduced disorder” is one that involves wanted or stimulated angiogenesis to treat a disease, disorder, and/or condition. The disorder is one characterized by tissue that is suffering from or be at risk of suffering from ischemic damage, infection, and/or poor healing, which results when the tissue is deprived of an adequate supply of oxygenated blood due to inadequate circulation. As used herein, “tissue” is used in the broadest sense, to include, but not limited to, the following: cardiac tissue, such as myocardium and cardiac ventricles; erectile tissue; skeletal muscle; neurological tissue, such as from the cerebellum; internal organs, such as the brain, heart, pancreas, liver, spleen, and lung; or generalized area of the body such as entire limbs, a foot, or distal appendages such as fingers or toes.

i. Methods of Vascularizing Ischemic Tissue

In one aspect in the method for the treatment of an angiogenesis reduced disorders, a compound of the invention may be used in a method of vascularizing ischemic tissue. As used herein, “ischemic tissue,” means tissue that is deprived of adequate blood flow. Examples of ischemic tissue include, but are not limited to, tissue that lack adequate blood supply resulting from mycocardial and cerebral infarctions, mesenteric or limb ischemia, or the result of a vascular occlusion or stenosis. In one example, the interruption of the supply of oxygenated blood may be caused by a vascular occlusion. Such vascular occlusion can be caused by arteriosclerosis, trauma, surgical procedures, disease, and/or other indications. There are many ways to determine if a tissue is at risk of suffering ischemic damage from undesirable vascular occlusion. Such methods are well known to physicians who treat such conditions. For example, in myocardial disease these methods include a variety of imaging techniques (e.g., radiotracer methodologies, x-ray, and MRI) and physiological tests. Therefore, induction of angiogenesis in tissue affected by or at risk of being affected by a vascular occlusion is an effective means of preventing and/or attenuating ischemia in such tissue. Thus, the treatment of skeletal muscle and myocardial ischemia, stroke, coronary artery disease, peripheral vascular disease, coronary artery disease are fully contemplated.

Any person skilled in the art of using standard techniques can measure the vascularization of tissue. Non-limiting examples of measuring vascularization in a subject include: SPECT (single photon emission computed tomography); PET (positron emission tomography); MRI (magnetic resonance imaging); and combination thereof, by measuring blood flow to tissue before and after treatment. Angiography can be used as an assessment of macroscopic vascularity. Histologic evaluation can be used to quantify vascularity at the small vessel level. These and other techniques are discussed in Simons, et al., “Clinical trials in coronary angiogenesis,” Circulation, 102, 73-86 (2000).

ii. Methods of Repairing Tissue

In one aspect in the method for the treatment of an angiogenesis reduced disorders, a compound of the present invention may be used in a method of repairing tissue. As used herein, “repairing tissue” means promoting tissue repair, regeneration, growth, and/or maintenance including, but not limited to, wound repair or tissue engineering. One skilled in the art readily appreciates that new blood vessel formation is required for tissue repair. In turn, tissue may be damaged by, including, but not limited to, traumatic injuries or conditions including arthritis, osteoporosis and other skeletal disorders, and burns. Tissue may also be damaged by results from injuries due to surgical procedures, irradiation, laceration, toxic chemicals, viral infection bacterial infection or burns. Tissue in need of repair also includes non-healing wounds. Non-limiting examples of non-healing wounds include: non-healing skin ulcers resulting from diabetic pathology; or fractures that do not heal readily.

Compounds of the invention may also be used in a method to aid in tissue repair in the context of guided tissue regeneration (GTR) procedures. Such procedures are currently used by those skilled in the medical arts to accelerate wound healing following invasive surgical procedures.

Compounds of the invention may be used in a method of promoting tissue repair characterized by enhanced tissue growth during the process of tissue engineering. As used herein, “tissue engineering” is defined as the creation, design, and fabrication of biological prosthetic devices, in combination with synthetic or natural materials, for the augmentation or replacement of body tissues and organs. Thus, the present method can be used to augment the design and growth of human tissues outside the body for later implantation in the repair or replacement of diseased tissues. For example, compounds of the invention may be useful in promoting the growth of skin graft replacements that are used as a therapy in the treatment of burns.

In another aspect of tissue engineering, compounds of the present invention may be included in cell-containing or cell-free devices that induce the regeneration of functional human tissues when implanted at a site that requires regeneration. As previously discussed, biomaterial-guided tissue regeneration can be used to promote bone regrowth in, for example, periodontal disease. Thus, an AMP may be used to promote the growth of reconstituted tissues assembled into three-dimensional configurations at the site of a wound or other tissue in need of such repair.

In another aspect of tissue engineering, compounds of the invention can be included in external or internal devices containing human tissues designed to replace the function of diseased internal tissues. This approach involves isolating cells from the body, placing them on or within structural matrices, and implanting the new system inside the body or using the system outside the body. The method of the invention can be included in such matrices to promote the growth of tissues contained in the matrices. For example, a compound can be included in a cell-lined vascular graft to promote the growth of the cells contained in the graft. It is envisioned that the method of the invention can be used to augment tissue repair, regeneration and engineering in products such as cartilage and bone, central nervous system tissues, muscle, liver, and pancreatic islet (insulin-producing) cells.

4. Vascular Tone Mediated Disorders

In one aspect of the invention, the PTPase mediated disorder is a vascular tone mediated disorder. As used herein, “vascular tone mediated disorder” is a disease or disorder that involves defects in endothelial PTK signaling thereby resulting in the biological manifestation of the disorder; in the biological cascade leading to the disorder; or as a symptom of the disorder. In one embodiment, the vascular tone mediated disorder is selected from the group consisting of primary essential hypertension, secondary hypertension, pulmonary hypertension and portal hypertension.

5. Vascular Permeability Mediated Disorders

In one aspect of the invention, the PTPase mediated disorder is a vascular permeability mediated disorder. As used herein, “vascular tone mediated disorder” is a disease or disorder that involves defects in VEGF induced vascular permeability thereby resulting in the biological manifestation of the disorder; in the biological cascade leading to the disorder; or as a symptom of the disorder. In one embodiment, the vascular permeability mediated disorder is selected from the group consisting of stroke, septic shock, burns, respiratory distress syndrome and congestive heart failure.

6. VEGF Mediated Disorders

In one aspect of the invention, the PTPase mediated disorder is a VEGF mediated disorder. As used herein, “VEGF mediated disorder” is a disease or disorder that involves defects in VEGF signaling thereby resulting in the biological manifestation of the disorder; in the biological cascade leading to the disorder; or as a symptom of the disorder. In one embodiment, the VEGF mediated disorder is selected from the group consisting of heart failure, myocardial infarction (MI), diabetic and ischemic neuropathy, osteoporosis, bone fracture healing, wound healing and hair loss.

A suitable MI cardiac pharmacological model is described in Mukherjee, R. et al., J. Cardiac Failure; 7 Suppl 2: 7 (2001). Briefly, pigs are prepared for the induction of myocardial infarction by implantation of an occlusion device on the circumflex coronary artery, and radiopaque markers are placed in the region destined to be infarcted to measure infarct expansion (see below). Measurements of left ventricular (hereinafter “LV”) volumes and distances between marker beads are made prior to and at various times after the induction of MI induced by activating the occlusion device.

The effects of compounds of the present invention effective in the treatment of MI may be studied in a pig model of MI induced by ligation of the circumflex coronary artery. Animals are assigned to one of the following treatment groups: (1) 1 or 10 mg/kg three times a day of a compound of Formula (I) by oral administration starting 3 days prior to myocardial infarction; (2) 10 mg/kg three times a day of said compound by oral administration starting 3 days after MI; (3) MI with no active treatment; or (4) no myocardial infarction or drug treatment. At 10 days post-MI, LV end-diastolic volume (hereinafter “LVEDV”) is measured by ventriculography. LVEDV is increased in all MI groups. An attenuated increase in LVEDV by a compound of Formula (I) indicates that the compound may be effective in the prevention or treatment of progressive ventricular dilation, and thus the subsequent development of CHF.

V. Compositions

The subject compounds can be administered as a composition that comprise: (a) a safe and effective amount of a compound of the invention; and (b) a pharmaceutically-acceptable carrier. The subject compositions may be useful for the treatment of PTPase mediated disorders.

A “safe and effective amount” of a subject compound is an amount that is effective, to treat a PTPase mediated disorder, without undue adverse side effects (such as toxicity, irritation, or allergic response), commensurate with a reasonable benefit/risk ratio when used in the manner of this invention. The specific “safe and effective amount” will vary with such factors as the particular condition being treated, the physical condition of the patient, the duration of treatment, the nature of concurrent therapy (if any), the specific dosage form to be used, the excipient employed, the solubility of the subject compound therein, and the dosage regimen desired for the composition. The term “pharmaceutically-acceptable carrier”, as used herein, means one or more compatible solid or liquid filler diluents or encapsulating substances which are suitable for administration to an animal, preferably a mammal, more preferably a human. The term “compatible”, as used herein, means that the components of the composition are capable of being commingled with the subject compound, and with each other, in a manner such that there is no interaction that would substantially reduce the pharmaceutical efficacy of the composition under ordinary use situations. Pharmaceutically-acceptable carriers must, of course, be of sufficiently high purity and sufficiently low toxicity to render them suitable for administration to the subject, preferably a mammal, more preferably a human being treated.

Some examples of substances which can serve as pharmaceutically-acceptable carriers or components thereof are: sugars, such as lactose, glucose and sucrose; starches; cellulose, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the Tweens®; wetting agents, such sodium lauryl sulfate; coloring agents; flavoring agents; tableting agents, stabilizers; antioxidants; preservatives; pyrogen-free water; isotonic saline; and phosphate buffer solutions.

The choice of a pharmaceutically-acceptable carrier to be used in conjunction with the subject compound is basically determined by the way the compound is to be administered.

In particular, pharmaceutically-acceptable carriers for systemic administration include sugars, starches, cellulose and its derivatives, malt, gelatin, talc, calcium sulfate, vegetable oils, synthetic oils, polyols, alginic acid, phosphate buffer solutions, emulsifiers, isotonic saline, and pyrogen-free water. Preferred carriers for parenteral administration include propylene glycol, ethyl oleate, pyrrolidone, ethanol, and sesame oil. Preferably, the pharmaceutically-acceptable carrier, in compositions for parenteral administration, comprises at least about 90% by weight of the total composition.

The compositions of this invention are preferably provided in unit dosage form. As used herein, a “unit dosage form” is a composition of this invention containing an amount of a subject compound that is suitable for administration to a subject according to good medical practice. These compositions preferably contain from about 5 mg (milligrams) to about 1000 mg, more preferably from about 10 mg to about 500 mg, more preferably from about 10 mg to about 300 mg, of a subject compound.

The compositions of this invention may be in any of a variety of forms, suitable, for example, for oral, rectal, topical, nasal, ocular or parenteral administration. Depending upon the particular route of administration desired, a variety of pharmaceutically-acceptable carriers well-known in the art may be used. These include solid or liquid fillers, diluents, hydrotropes, surface-active agents, and encapsulating substances. Optional pharmaceutically-active materials may be included, which do not substantially interfere with the inhibitory activity of the subject compound. The amount of carrier employed in conjunction with the subject compound is sufficient to provide a practical quantity of material for administration per unit dose of the subject compound. Techniques and compositions for making dosage forms useful in the methods of this invention are described in the following references, all incorporated by reference herein: Modern Pharmaceutics, Chapters 9 and 10 (Banker & Rhodes, editors, 1979); Lieberman et al., Pharmaceutical Dosage Forms: Tablets (1981); and Ansel, Introduction to Pharmaceutical Dosage Forms 2d Edition (1976).

Various oral dosage forms can be used, including such solid forms as tablets, capsules, granules and bulk powders. These oral forms comprise a safe and effective amount, usually at least about 5%, and preferably from about 25% to about 50%, of the Formula (I) compound. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, and containing suitable solvents, preservatives, emulsifying agents, suspending agents, diluents, sweeteners, melting agents, coloring agents and flavoring agents.

The pharmaceutically-acceptable carrier suitable for the preparation of unit dosage forms for peroral administration are well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical for the purposes of the subject invention, and can be readily made by a person skilled in the art.

Peroral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, Avicel® RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit® coatings, waxes and shellac.

Other compositions useful for attaining systemic delivery of the subject compounds include sublingual, buccal, suppository, and nasal dosage forms.

The compositions of this invention can also be administered topically to a subject, e.g., by the direct laying on or spreading of the composition on the epidermal or epithelial tissue of the subject, or transdermally via a “patch”. Such compositions include, for example, lotions, creams, solutions, gels and solids. These topical compositions preferably comprise a safe and effective amount, usually at least about 0.1%, and preferably from about 1% to about 5%, of the Formula (I) compound. Suitable carriers for topical administration preferably remain in place on the skin as a continuous film, and resist being removed by perspiration or immersion in water. Generally, the carrier is organic in nature and capable of having dispersed or dissolved therein the Formula (I) compound. The carrier may include pharmaceutically-acceptable emollients, emulsifiers, thickening agents, solvents and the like.

The specific dosage of subject compound or composition to be administered, as well as the duration of treatment, are mutually dependent. The dosage and treatment regimen will also depend upon such factors as the specific subject compounds used, the specific PTPase mediated disorder, the ability of the subject compound to reach minimum inhibitory concentrations at the site of the disorder, the nature and extent of other disorder (if any), the personal attributes of the subject (such as weight), compliance with the treatment regimen, the age and health status of the patient, and the presence and severity of any side effects of the treatment.

Typically, for a human adult (weighing approximately 70 kilograms), from about 75 mg, more preferably from about 200 mg, most preferably from about 500 mg to about 30,000 mg, more preferably to about 10,000 mg, most preferably to about 3,500 mg, of a subject compound is administered per day. Treatment regimens preferably extend from about 1, preferably from about 3 to about 56 days, preferably to about 20 days, in duration. Prophylactic regimens (such as prevention of osteoporosis) may extend 6 months, or longer, according to good medical practice.

### VI. EXAMPLES

The R groups used to illustrate the compound examples of this section VI may not correlate to the respective R group used to describe the various moieties of Formula (J).

### Examples 1-27

The following chemical formula along with Table 1 shows the structure of compounds made according to the description in Examples 1-27 described below.

TABLE 1

| | |
|---|---|
| | Formula (III) |
| 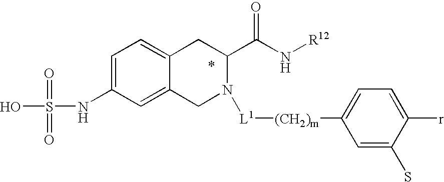 | |

| EXAMPLE | * | L¹ | m | R¹² | s | r |
|---|---|---|---|---|---|---|
| 1 | S | —CO₂— | 1 | —CH₃ | Nil | Nil |
| 2 | S | —CO— | 2 | —CH₃ | Nil | Nil |
| 3 | S | —CONH— | 1 | —CH₃ | Nil | Nil |
| 4 | S | —SO₂— | 1 | —CH₃ | Nil | Nil |
| 5 | S | —CO— | 2 | —CH₃ | Nil | —CF₃ |
| 6 | S | —CO— | 3 | —CH₃ | Nil | Nil |
| 7 | S | —CO— | 4 | —CH₃ | Nil | Nil |
| 8 | S | —CO— | 2 | —CH₃ | —NH—SO₂—OH | Nil |
| 9 | S | —CO— | 2 | —CH₃ | Nil | —CH₃ |
| 10 | S | —CO— | 2 | —CH₃ | —OH | Nil |
| 11 | S | —CO— | 2 | —CH₃ | Nil | —O—CH₃ |
| 12 | S | —CO— | 0 | 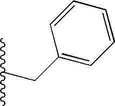 | Nil | 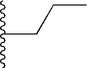 |
| 13 | S | —CO— | 2 | 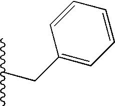 | Nil | Nil |
| 14 | S | —CO₂— | 1 | 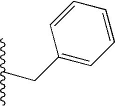 | Nil | Nil |
| 15 | S | —CO— | 2 | —CH₃ | —SO₂—NH₂ | Nil |
| 16 | S | —CO— | 2 | —CH₃ | 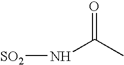 | Nil |
| 17 | S | —CO— | 2 | —CH₃ | 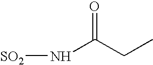 | Nil |
| 18 | S | —CO— | 2 | —CH₃ | 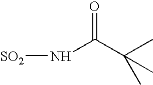 | Nil |
| 19 | S | —CO— | 2 | —CH₃ | 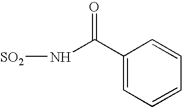 | Nil |
| 20 | S | —CO— | 2 | —CH₃ | Nil | 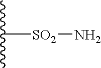 |
| 21 | S | —CO— | 2 | —CH₃ | Nil | 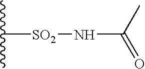 |
| 22 | R | —CO— | 2 | —CH₃ | 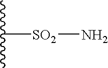 | Nil |
| 23 | R | —CO— | 2 | —CH₃ | 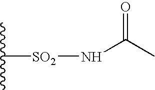 | Nil |
| 24 | S | —CO— | 2 | —CH₃ | 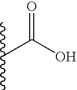 | Nil |
| 25 | S | —CO— | 2 | 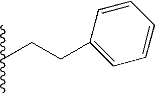 | 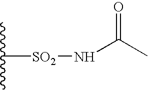 | Nil |
| 26 | S | —CO— | 0 | —CH₃ | Nil | Nil |
| 27 | S | —CO— | 2 | —CH₃ | —Cl | Nil |

### Example 1

### (S)-3-Methylcarbamoyl-7-sulfoamino-3,4-dihydro-1H-isoquinoline-2-carboxylic Acid Tert-Butyl Ester

(S)-7-Nitro-3,4-dihydro-1H-isoquinoline-2,3-dicarboxylic acid 2-tert-butyl ester (10.0 g, 31.1 mmol) is slurried in tetrahydrofuran (THF) (20 mL). N-Methylmorpholine (3.75 mL, 34.2 mmol) is added to the solution and stirred 10 min at which point the solution turned homogeneous. The dark brown solution is cooled to 0° C. and isobutyl chloroformate (4.65 mL, 34.2 mmol) is slowly added over a period of 10 min and the reaction is stirred at 0° C. for 30 min. Methylamine in THF (2M, 78.0 mL, 155.3 mmol) is slowly added over a period of 30 min, stirred at 0° C. for 6 h and allowed to warm to room temperature gradually over 18 h. The solvent is evaporated, ethyl acetate (500 mL) is added and the organic layer is washed with water (5×200 mL). The organic layer is dried over magnesium sulfate, filtered and evaporated to dryness to give a brown-orange oil. The crude product is purified by flash column chromatography (19:1 chloroform/methanol) to give an off white solid. Yield 8.03 g (77%). ¹H NMR (CDCl₃) δ 7.99 (m, 2H), 7.93 (s, 1H), 4.72 (m, 2H), 4.42 (s, 1H), 3.20 (m, 2H), 2.68 (m, 3H), 1.42 (s, 9H). ¹³C {¹H} NMR (CDCl₃) δ 175.7, 156.1, 147.0, 141.9, 134.6, 129.8, 122.7, 121.6, 82.3, 77.7, 44.8, 30.7, 28.7, 26.7. ESI-MS (m/z): 334[M−H]⁻.

### (S)-3-Methylcarbamoyl-7-nitro-1,2,3,4-tetrahydro-isoquinoline HCl

(S)-3-Methylcarbamoyl-7-nitro-3,4-dihydro-1H-isoquinoline-2-carboxylic acid tert-butyl ester (7.0 g, 20.9 mmol) is dissolved in 4M HCl in dioxane (20 mL) and stirred for 30 min. The solvent is evaporated and 4M HCl in Dioxane (20 mL) is added again and stirred for 30 min. The solvent is evaporated to dryness to give a brown-orange powder. Yield 5.21 g (92%). ¹H NMR (D₂O) δ 8.08 (m, 2H), 7.43 (s, 1H), 4.53 (s, 2H), 4.30 (m, 1H), 3.34 (m, 2H), 2.73 (s, 3H). ¹³C {¹H} NMR (D2O) δ 175.7, 147.1, 138.4, 130.5, 129.2, 123.4, 122.4, 66.9, 44.2, 29.6, 26.3. ESI-MS (m/z): 236[M+H]⁺.

### (S)-3-Methylcarbamoyl-7-sulfoamino-3,4-dihydro-1H-isoquinoline-2-carboxylic Acid Benzyl Ester

(S)-3-Methylcarbamoyl-7-nitro-1,2,3,4-tetrahydro-isoquinoline hydrochloride (0.200 g, 0.74 mmol) is dissolved in 1,4-dioxane (3 mL). A solution of sodium carbonate (0.157 g, 1.5 mmol) in water (2 mL) is added to the 1,4-dioxane solution and the mixture is cooled to 0° C. Benzylchloroformate (0.10 mL, 0.7 mmol) is added and the reaction mixture is taken out of the cooling bath and stirred for 1 h. Ethyl acetate is added (50 mL) and the organic layer is washed with water (3×25 mL). The organic layer is dried over magnesium sulfate, filtered and evaporated to give a white solid. Yield 0.26 g (95%). ESI-MS (m/z): 368[M−H]⁻. The product is dissolved in 1:1 ethyl acetate: ethanol (5 mL) and tin(II) chloride dihydrate (1.59 g, 7.0 mmol) is added and the reaction mixture is heated to 50° C. for 3 h. Ethyl acetate (30 mL) is added and the solution is washed with 1N sodium hydroxide (3×20 mL). The organic layer is dried over magnesium sulfate, filtered and evaporated to give a yellow solid. Yield 0.13 g (54%). ESI-MS (m/z): 338[M−H]⁻. The product is dissolved in pyridine (3 mL) and sulfur trioxide pyridine complex (0.189 g, 1.2 mmol) is added to the stirring solution. The resulting heterogeneous solution is stirred for 5 min. then quenched with 7% ammonium hydroxide (aq) (25 mL) and stirred for an additional 5 min. The solvents are evaporated to dryness and the residue is dissolved in 7% ammonium hydroxide (aq) (25 mL) and evaporated to dryness. The resulting solid is HPLC purified to give 0.090 g (54%) (29% over 3 steps) of an off-white solid.

Mixture of rotational isomers. ¹H NMR (D₂O) δ 7.25 (m, 5H), 6.95 (m, 3H), 5.10 (m, 2H), 4.40 (m, 3H), 3.0 (m, 2H), 2.27 (d, 3H). ¹³C {¹H} NMR (D₂O) δ 175.37, 174.82, 157.77, 157.25, 139.20, 136.45, 136.31, 135.06, 134.65, 129.20, 129.10, 128.91, 128.83, 128.78, 128.55, 128.34, 128.24, 128.08, 118.88, 118.78, 117.44, 117.36, 68.50, 68.34, 56.96, 56.70, 45.28, 45.19, 31.37, 31.11, 26.10, 26.01. ESI-MS (m/z): 418[M−H]⁻. Anal. Calcd. for C₁₉H₂₁N₃O₆S.1.33H₂O 2/3 NH₃: C, 50.17; H, 5.60; N, 11.59. Found: C, 50.17; H, 5.69; N, 11.29.

### Example 2

### [(S)-3-Methylcarbamoyl-2-(3-phenyl-propionyl)-1,2,3,4-tetrahydro-isoquinolin-7-yl]-sulfamic Acid

(S)-3-Methylcarbamoyl-7-nitro-1,2,3,4-tetrahydro-isoquinoline hydrogen chloride (0.30 g, 1.1 mmol) is slurried in dichloromethane (3 mL) and diisopropylethylamine (0.290 mL, 1.7 mmol) is added to the stirring solution. Hydrocinnamoyl chloride (0.116 mL, 1.3 mmol) is added and the reaction is stirred for 2 h. Ethyl acetate (20 mL) is added and the solution is washed with 1N sodium hydroxide (3×10 mL), water (10 mL), 0.1N hydrochloric acid (3×10 mL) and brine (10 mL). The organic layer is dried over magnesium sulfate, filtered and evaporated to give a yellow solid. Yield 0.365 g (90%). ESI-MS (m/z): 366[M−H]⁻. The product is dissolved in ethanol (3 mL) and degassed with argon for 2 min. 10% Palladium on carbon (0.08 g) is added and hydrogen is bubbled through the stirring solution for 3 h. The reaction mixture is filtered through celite and the solvent is evaporated to give a yellow solid. Yield 0.283 g (85%). ESI-MS (m/z): 336[M−H]⁻. The product is dissolved in pyridine (3 mL) and sulfur trioxide pyridine complex (0.368 g, 2.3 mmol) is added to the stirring solution. The resulting heterogeneous solution is stirred for 5 min. then quenched with 7% ammonium hydroxide (aq) (25 mL) and the solution is stirred for 5 min. The solvents are evaporated to dryness and the residue is redissolved in 7% ammonium hydroxide (aq) (25 mL) and evaporated to dryness. The resulting solid is HPLC purified to give 0.135 g (37%) (28% over 3 steps) of an off-white solid.

Mixture of rotational isomers. ¹H NMR (D₂O) δ 7.20 (m, 6H), 6.95 (m, 2H), 4.40 (m, 2H), 4.20 (m, 1H), 2.80 (m, 6H), 2.43 (d, 3H). ¹³C {¹H} NMR (D₂O) δ 177.64, 177.55, 175.74, 175.13, 142.41, 142.21, 140.75, 140.64, 135.53, 135.43, 130.60, 130.52, 130.25, 130.06, 129.58, 128.95, 128.43, 128.31, 120.23, 120.06, 118.88, 118.65, 58.83, 57.22, 48.10, 46.00, 36.85, 36.62, 33.00, 32.84, 32.49, 32.22, 27.68, 27.56. ESI-MS (m/z): 415[M−H]⁻. Anal. Calcd. for C₂₀H₂₆N₄O₅S.1/2H₂O: C, 54.16; H, 6.14; N, 12.63. Found: C, 54.18; H, 6.02; N, 12.68.

### Example 3

### [(S)-2-Benzylcarbamoyl-3-methylcarbamoyl-1,2,314-tetrahydro-isoquinolin-7-yl]-sulfamic Acid

(S)-3-Methylcarbamoyl-7-nitro-1,2,3,4-tetrahydro-isoquinolin hydrochloride (0.20 g, 0.74 mmol) is slurried in dichloromethane: pyridine 1:1 (3 mL) and benzyl isocyanate (0.10 mL, 0.7 mmol) is added and the reaction is stirred for 2 h. Ethyl acetate (20 mL) is added and the organic layer is washed with water (3×10 mL) and brine (10 mL). The organic layer is dried over magnesium sulfate, filtered and evaporated to give a yellow solid. Yield 0.260 g (95%). ESI-MS (m/z): 368[M−H]⁻. The product is dissolved in ethanol (3 mL) and degassed with argon for 2 min. 10% Palladium on carbon (0.08 g) is added and hydrogen is bubbled through the stirring solution for 3 h. The reaction mixture is filtered through celite and the solvent is evaporated to dryness to give yellow oil. Yield 0.230 g (96%). ESI-MS (m/z): 338[M−H]⁻. The product is dissolved in pyridine (3 mL) and sulfur trioxide pyridine complex (0.325 g, 2.1 mmol) is added to the stirring solution. The resulting heterogeneous solution is stirred for 5 min then quenched with 7% ammonium hydroxide (aq) (25 mL) and stirred for an additional 5 min. The solvents are evaporated to dryness and the residue is re-dissolved in 7% ammonium hydroxide (aq) (25 mL) and evaporated to dryness. The resulting solid is HPLC purified to give 0.090 g (30%) (28% over 3 steps) of an off-white solid. ¹H NMR (D₂O) δ 7.28 (m, 6H), 7.00 (m, 2H), 4.50 (m, 1H), 4.35 (m, 4H), 3.05 (m, 2H), 2.39 (s, 3H). ¹³C {¹H} NMR (D₂O) δ 175.43, 159.78, 139.92, 139.12, 134.07, 129.04, 128.77, 128.19, 127.52, 127.32, 118.81, 117.44, 55.77, 45.24, 44.31, 31.36, 26.06. ESI-MS (m/z): 418[M−H]⁻. Anal. Calcd. for C₁₉H₂₅N₅O₅S.2/3H₂O: C, 50.99; H, 5.93; N, 15.65. Found: C, 50.96; H, 5.80; N, 15.52.

### Example 4

### (S)-(3-Methylcarbamoyl-2-phenylmethanesulfonyl-1,2,3,4-tetrahydro-isoquinolin-7-yl)-sulfamic Acid

(S)-3-Methylcarbamoyl-7-nitro-1,2,3,4-tetrahydro-isoquinoline hydrochloride (0.20 g, 0.7 mmol) is slurried in dichloromethane (3 mL) and diisopropyl ethylamine (0.400 mL, 2.1 mmol) is added to the stirring solution. α-Toluenesufonyl chloride (0.161 mL, 0.8 mmol) is added and the reaction is stirred for 30 min. Ethyl acetate (20 mL) is added and the solution is washed with 1N sodium hydroxide (3×10 mL), water (10 mL), 0.1N hydrochloric acid (3×10 mL) and brine (10 mL). The organic layer is dried over magnesium sulfate, filtered and evaporated to give a yellow solid. Yield 0.250 g (87%). ESI-MS (m/z): 390[M+H]⁺. The product is dissolved in ethanol (3 mL) and degassed with argon for 2 min. 10% Palladium on carbon (0.08 g) is then added and hydrogen is bubbled through the stirring solution for 3 h. The reaction mixture is filtered through celite and the solvent is evaporated to give a yellow solid. Yield 0.217 g (82%). ESI-MS (m/z): 358[M−H]⁻. The product is dissolved in pyridine (3 mL) and sulfur trioxide pyridine complex (0.288 g, 1.8 mmol) is added to the stirring solution. The resulting heterogeneous solution is stirred for 5 min then quenched with 7% ammonium hydroxide (aq) (25 mL) and stirred for an additional 5 min. The solvents are evaporated to dryness and the residue is redissolved in 7% ammonium hydroxide (aq) (25 mL) and evaporated to dryness. The resulting solid is HPLC purified to give 0.170 g (64%) (52% over 3 steps) of an off-white solid.

¹H NMR (D₂O) δ 7.30-6.91(m, 8H), 4.37-4.17(m, 4H), 3.92 (t, 1H, J=6 Hz), 2.77 (m, 1H), 2.45 (s, 3H). ¹³C {(H} NMR (D₂O) δ 174.41, 139.38, 134.44, 131.07, 129.38, 129.29, 129.10, 127.95, 127.62, 118.86, 116.93, 57.59, 56.24, 46.14, 30.83, 26.19. ESI-MS (m/z): 438[M−H]⁻. Anal. Calcd. for C₁₈H₂₄N₄O₆S₂.2/3H₂O: C, 46.14; H, 5.45; N, 11.96. Found: C, 46.32; H, 5.04; N, 11.76.

### Example 5

### (S)-{3-Methylcarbamoyl-2-[3-(4-trifluoromethyl-phenyl)-propionyl]-1,2,3,4-tetrahydro-isoquinolin-7-yl}-sulfamic Acid

4-(trifluoromethyl)hydrocinnamic acid (0.251 g, 1.2 mmol) is dissolved in dichloromethane (3 mL). EDC (0.221, 1.2 mmol) is added and the reaction is stirred for 30 min. Diisopropyl ethylamine (0.400 mL, 2.2 mmol) is added to the solution followed by (S)-3-methylcarbamoyl-7-nitro-1,2,3,4-tetrahydro-isoquinoline hydrochloride (0.20 g, 0.74 mmol) and the reaction is stirred for 1 h. Ethyl acetate (20 mL) is added and the solution is washed with 1N sodium hydroxide (3×10 mL), water (10 mL), 0.1N hydrochloric acid (3×10 mL) and brine (10 mL). The organic layer is dried over magnesium sulfate, filtered and evaporated to give a yellow solid. Yield 0.259 g (80%). ESI-MS (m/z): 434[M−H]⁻. The product is dissolved in ethanol (3 mL) and degassed with argon for 2 min. 10% Palladium on carbon (0.08 g) is added and hydrogen is bubbled through the stirring solution for 3 h. The reaction mixture is filtered through celite and the solvent is evaporated to give a brown solid. Yield 0.232 g (96%). ESI-MS (m/z): 404[M−H]⁻. The product is dissolved in pyridine (3 mL) and sulfur trioxide pyridine complex (0.273 g, 1.8 mmol) is added to the stirring solution. The resulting heterogeneous solution is stirred for 5 min then quenched with 7% ammonium hydroxide (aq) (25 mL) and the solution is stirred for an additional 5 min. The solvents are evaporated to dryness and the residue is re-dissolved in 7% ammonium hydroxide (aq) (25 mL) and evaporated to dryness. The resulting solid is HPLC purified to give 0.155 g (54%) (42% over 3 steps) of an off-white solid.

Mixture of rotational isomers. ¹H NMR (D₂O) δ 7.20 (m, 2H), 7.05 (m, 2H), 6.89 (m, 3H), 4.40 (m, 2H), 4.07 (m, 1H), 2.87 (m, 2H), 2.79-2.52 (m, 4H), 2.35 (s, 3H). ³C{¹H} NMR (D₂O) δ 175.23, 174.91, 174.04, 172.97, 145.31, 145.07, 139.35, 133.62, 129.08, 128.78, 128.68, 128.33, 128.20127.92, 127.72, 126.95, 126.40, 125.42, 122.80, 118.57, 118.41, 117.18, 117.06, 56.94, 55.36, 46.35, 44.31, 34.68, 31.18, 30.98, 30.70, 30.58, 26.19, 26.06. ESI-MS (m/z): 484[M−H]⁻. Anal. Calcd. For C₂₁H₂₅N₄O₅S.1/2H₂O: C, 49.31; H, 5.12; N, 10.95. Found: C, 49.02; H, 4.76; N, 10.73.

### Example 6

### (S)-[3-Methylcarbamoyl-2-(4-phenyl-butyryl)-1,2,3,4-tetrahydro-isoquinolin-7-yl]-sulfamic Acid

4-Phenylbutyric acid (0.189 g, 1.2 mmol) is dissolved in dichloromethane (3 mL) and EDC (0.221, 1.2 mmol) is added. The reaction is stirred for 30 min. Diisopropyl ethylamine (0.400 mL, 2.2 mmol) is added to the stirring solution followed by (S)-3-methylcarbamoyl-7-nitro-1,2,3,4-tetrahydro-isoquinoline hydrochloride (0.20 g, 0.74 mmol) and the reaction is stirred for 1 h. Ethyl acetate (20 mL) is added and the solution is washed with 1N sodium hydroxide (3×10 mL), water (10 mL), 0.1N hydrochloric acid (3×10 mL) and brine (10 mL). The organic layer is dried over magnesium sulfate, filtered and evaporated to give a yellow solid. Yield 0.258 g (92%). ESI-MS (m/z): 380[M−H]⁻. The product is dissolved in ethanol (3 mL) and degassed with argon for 2 min. 10% Palladium on carbon (0.08 g) is added and hydrogen is bubbled through the stirring solution for 3 h. The reaction mixture is filtered through celite and the solvent is evaporated to give a brown solid. Yield 0.232 g (98%). ESI-MS (m/z): 350[M−H]⁻. The product is dissolved in pyridine (3 mL) and sulfur trioxide pyridine complex (0.328 g, 2.1 mmol) is added to the stirring solution. The resulting heterogeneous solution is stirred for 5 min then quenched with 7% ammonium hydroxide (aq) (25 mL) and the solution is stirred for an additional 5 min. The solvents are evaporated to dryness and the residue is re-dissolved in 7% ammonium hydroxide (aq) (25 mL) and evaporated to dryness. The resulting solid is HPLC purified to give 0.175 g (53%) (47% over 3 steps) of an off-white solid.

Mixture of rotational isomers ¹H NMR (D₂O) δ 7.40-7.01 (m, 8H), 4.74-4.40 (m, 3H), 3.05 (m, 2H), 2.75-2.43 (m, 7H), 1.93 (m, 2H). ¹³C {¹H} NMR (D₂O) δ 176.85, 174.55, 173.80, 142.27, 142.02, 139.27, 139.18, 134.38, 134.10, 128.99, 128.57, 128.41, 127.66, 126.52, 118.90, 118.65, 117.52, 117.25, 57.36, 55.94, 46.62, 44.62, 34.64, 34.48, 32.89, 31.78, 30.93, 26.39, 26.13, 26.04. ESI-MS (m/z): 484[M−H]⁻. Anal. Calcd. for C₂₁H₂₈N₄O₅S_{5/9}H₂O: C, 55.01; H, 6.40; N, 12.22. Found: C, 54.67; H, 5.83; N, 11.94.

### Example 7

### (S)-[3-Methylcarbamoyl-2-(5-phenyl-pentanoyl)-1,2,3,4-tetrahydro-isoquinolin-7-yl]-sulfamic Acid

5-Phenylvaleric acid (0.205 g, 1.2 mmol) is dissolved in dichloromethane (3 mL) and EDC (0.221, 1.2 mmol) is added. The reaction is stirred for 30 min. Diisopropyl ethylamine (0.400 mL, 2.2 mmol) is added to the stirring solution followed by (S)-3-methylcarbamoyl-7-nitro-1,2,3,4-tetrahydro-isoquinoline hydrochloride (0.20 g, 0.74 mmol) and the reaction is stirred for 1 h. Ethyl acetate (20 mL) is added and the solution is washed with 1N sodium hydroxide (3×10 mL), water (10 mL), 0.1N hydrochloric acid (3×10 mL) and brine (10 mL). The organic layer is dried over magnesium sulfate, filtered and evaporated to give a yellow solid. Yield 0.274 g (94%). ESI-MS (m/z): 394[M−H]⁻. The product is dissolved in ethanol (3 mL) and degassed with argon for 2 min. 10% Palladium on carbon (0.08 g) is then added and hydrogen is bubbled through the stirring solution for 3 h. The reaction mixture is filtered through celite and the solvent is evaporated to give a yellow solid. Yield 0.242 g (96%). ESI-MS (m/z): 364[M−H]⁻. The product is dissolved in pyridine (3 mL) and sulfur trioxide pyridine complex (0.292 g, 2.1 mmol) is added to the stirring solution. The resulting heterogeneous solution is stirred for 5 min then quenched with 7% ammonium hydroxide (aq) (25 mL) and the solution is stirred for an additional 5 min. The solvents are evaporated to dryness and the residue is re-dissolved in 7% ammonium hydroxide (aq) (25 mL) and evaporated to dryness. The resulting solid is HPLC purified to give 0.129 g (46%) (39% over 3 steps) of an off-white solid.

Mixture of rotational isomers. ¹H NMR (D₂O) δ 7.30-6.89 (m, 8H), 4.46 (m, 3H), 2.97 (m, 2H), 2.59-2.35 (m, 7H), 1.52 (m, 4H). ESI-MS (m/z): 444[M−H]⁻. Anal. Calcd. for C₂₂H₃₀N₄O₅S.2/3H₂O: C, 56.03; H, 6.63; N, 11.88. Found: C, 55.72; H, 6.76; 11.86.

### Example 8

### (S)-{3-Methylcarbamoyl-2-[3-(3-sulfoamino-phenyl)-propionyl]-1,2,3,4-tetrahydro-isoquinolin-7-yl}-sulfamic Acid

3-(3-Nitrophenyl)propionic acid (0.225 g, 1.2 mmol) is dissolved in dichloromethane (3 mL) and EDC (0.221, 1.2 mmol) is added. The reaction is stirred for 30 min. Diisopropyl ethylamine (0.400 mL, 2.2 mmol) is added to the stirring solution followed by (S)-3-methylcarbamoyl-7-nitro-1,2,3,4-tetrahydro-isoquinoline hydrochloride (0.20 g, 0.74 mmol) and the reaction is stirred for 1 h. Ethyl acetate (20 mL) is added and the solution is washed with 1N sodium hydroxide (3×10 mL), water (10 mL), 0.1N hydrochloric acid (3×10 mL) and brine (10 mL). The organic layer is dried over magnesium sulfate, filtered and evaporated to give a yellow solid. Yield 0.252 g (83%). ESI-MS (m/z): 411[M−H]⁻. The product is dissolved in ethanol (3 mL) and degassed with argon for 2 min. 10% Palladium on carbon (0.08 g) is added and hydrogen is bubbled through the stirring solution for 3 h. The reaction mixture is filtered through celite and the solvent is evaporated to give a yellow solid. Yield 0.210 g (97%). ESI-MS (m/z): 351[M−H]⁻. The product is dissolved in pyridine (3 mL) and sulfur trioxide pyridine complex (0.607 g, 3.2 mmol) is added to the stirring solution. The resulting heterogeneous solution is stirred for 5 min then quenched with 7% ammonium hydroxide (aq) (25 mL) and the solution is stirred for an additional 5 min. The solvents are evaporated to dryness and the residue is re-dissolved in 7% ammonium hydroxide (aq) (25 mL) and evaporated to dryness. The resulting solid is HPLC purified to give 0.090 g (28%) (22% over 3 steps) of an off-white solid.

Mixture of rotational isomers. ¹H NMR (D₂O) δ 7.12-6.69 (m, 7H), 4.50-4.18 (m, 3H), 2.91-2.68 (m, 6H), 2.35 (d, 3H). 13C {¹H} NMR (D₂O) δ 176.35, 176.26, 174.40, 173.81, 142.17, 141.92, 140.58, 140.49, 139.18, 139.05, 134.14, 130.00, 129.87, 128.56, 128.39, 127.76, 123.28, 123.20, 119.13, 118.86, 118.70, 117.56, 117.43, 117.28, 57.45, 55.90, 46.72, 44.68, 35.39, 35.04, 31.62, 31.13, 30.81, 26.21, 26.08. ESI-MS (m/z): 511[M−H]⁻. Anal. Calcd. for C₂₀H₂₇N₅O₈S₂.1H₂O: C, 42.54; H, 5.71; N, 14.88. Found: C, 42.28; H, 5.77; N, 14.90.

### Example 9

### (S)-[3-Methylcarbamoyl-2-(3-p-tolyl-propionyl)-1,2,3,4-tetrahydro-isoquinolin-7-yl]-sulfamic Acid

3-(p-Tolyl)propionic acid (0.189 g, 1.2 mmol) is dissolved in dichloromethane (3 mL) and EDC (0.221, 1.2 mmol) is added. The reaction is stirred for 30 min. Diisopropyl ethylamine (0.400 mL, 2.2 mmol) and (S)-3-methylcarbamoyl-7-nitro-1,2,3,4-tetrahydro-isoquinoline hydrochloride (0.20 g, 0.74 mmol) are added and the reaction is stirred for 1 h. Ethyl acetate (20 mL) is added and the solution is washed with 1N sodium hydroxide (3×10 mL), water (10 mL), 0.1N hydrochloric acid (3×10 mL) and brine (10 mL). The organic layer is dried over magnesium sulfate, filtered and evaporated to give a yellow solid. Yield 0.205 g (73%). ESI-MS (m/z): 380[M−H]⁻. The product is dissolved in ethanol (3 mL) and degassed with argon for 2 min. 10% Palladium on carbon (0.08 g) is then added and hydrogen is bubbled through the stirring solution for 3 h. The reaction mixture is filtered through celite and the solvent is evaporated to give a yellow solid. Yield 0.185 g (98%). ESI-MS (m/z): 350[M−H]⁻. The product is dissolved in pyridine (3 mL) and sulfur trioxide pyridine complex (0.261 g, 1.5 mmol) is added to the stirring solution. The resulting heterogeneous solution is stirred for 5 min then quenched with 7% ammonium hydroxide (aq) (25 mL) and the solution is stirred for an additional 5 min. The solvents are evaporated to dryness and the residue is re-dissolved in 7% ammonium hydroxide (aq) (25 mL) and evaporated to dryness. The resulting solid is HPLC purified to give 0.095 g (40%) (39% over 3 steps) of an off-white solid.

Mixture of rotational isomers. ¹H NMR (D₂O) δ 7.16-6.92 (m, 7H), 4.564.28 (m, 3H), 3.07-2.74 (m, 6H), 2.50 (d, 3H), 2.21 (d, 3H). ¹³C {¹H} NMR (D₂O) δ 176.37, 174.20, 173.81, 139.22, 139.07, 137.60, 137.46, 136.88, 134.09, 133.94, 129.68, 129.60, 128.73, 128.56, 128.05, 127.71, 118.76, 118.68, 117.46, 117.17, 57.47, 55.62, 46.64, 44.47, 35.54, 35.19, 31.48, 31.12, 30.80, 30.61, 26.19, 26.07, 20.36. ESI-MS (m/z): 430[M−H]⁻. Anal. Calcd. for C₂₁H₂₈N₄O₅S.2/3H₂O: C, 54.77; H, 6.42; N, 12.17. Found: C, 54.72; H, 6.33; N, 12.21.

### Example 10

### (S)-{2-[3-(3-Hydroxy-phenyl)-propionyl]-3-methylcarbamoyl-1,2,3,4-tetrahydro-isoquinolin-7-yl}-sulfamic Acid

3-(3-Hydroxyphenyl)propionic acid (0.249 g, 1.2 mmol) is dissolved in dichloromethane (3 mL). EDC (0.221, 1.2 mmol) is added and the reaction is stirred for 30 min. Diisopropyl ethylamine (0.400 mL, 2.2 mmol) is added to the stirring solution followed by (S)-3-methylcarbamoyl-7-nitro-1,2,3,4-tetrahydro-isoquinoline hydrochloride (0.20 g, 0.74 mmol) and the reaction is stirred for 1 h. Ethyl acetate (20 mL) is added and the solution is washed with 1N sodium hydroxide (3×10 mL), water (10 mL), 0.1N hydrochloric acid (3×10 mL) and brine (10 mL). The organic layer is dried over magnesium sulfate, filtered and evaporated to give a yellow solid. Yield 0.191 g (67%). ESI-MS (m/z): 382[M−H]⁻. The product is dissolved in ethanol (3 mL) and degassed with argon for 2 min. 10% Palladium on carbon (0.08 g) is added and hydrogen is bubbled through the stirring solution for 3 h. The reaction mixture is filtered through celite and the solvent is evaporated to give a yellow solid. Yield 0.140 g (80%). ESI-MS (m/z): 352[M−H]⁻. The product is dissolved in pyridine (3 mL) and sulfur trioxide pyridine complex (0.189 g, 1.2 mmol) is added to the stirring solution. The resulting heterogeneous solution is stirred for 5 min then quenched with 7% ammonium hydroxide (aq) (25 mL) and the solution is stirred for an additional 5 min. The solvents are evaporated to dryness and the residue is re-dissolved in 7% ammonium hydroxide (aq) (25 mL) and evaporated to dryness. The resulting solid is HPLC purified to give 0.040 g (24%) (12% over 3 steps) of an off-white solid.

Mixture of rotational isomers. ¹H NMR (D₂O) δ 7.10-6.49 (m, 7H), 4.494.17 (m, 3H), 2.97-2.68 (m, 6H), 2.40 (d, 3H). ¹³C {¹H} NMR (D₂O) δ 176.32, 174.20, 173.77, 155.85, 142.76, 139.22, 139.01, 133.94, 130.55, 130.45, 128.58, 128.12, 127.64, 120.91, 118.82, 118.69, 117.50, 117.25, 115.51, 113.84, 113.66, 57.52, 55.64, 46.68, 44.60, 35.30, 34.96, 31.53, 31.42, 31.23, 30.62, 26.19, 26.09. ESI-MS (m/z): 432[M−H]⁻. Anal. Calcd. for C₂₀H₂₆N₄O₆S.1/2H₂O: C, 52.28; H, 5.92; N, 12.19. Found: C, 52.35; H, 5.59; N, 12.08.

### Example 11

### (S)-{2-[3-(4-Methoxy-phenyl)-propionyl]-3-methylcarbamoyl-1,2,3,4-tetrahydro-isoquinolin-7-yl}-sulfamic Acid

3-(4-Methoxyphenyl)propionic acid (0.207 g, 1.2 mmol) is dissolved in dichloromethane (3 mL). EDC (0.221, 1.2 mmol) is added and the reaction is stirred for 30 min. Diisopropyl ethylamine (0.400 mL, 2.2 mmol) is added to the stirring solution followed by (S)-3-methylcarbamoyl-7-nitro-1,2,3,4-tetrahydro-isoquinoline hydrochloride (0.20 g, 0.74 mmol) and the reaction is stirred for 1 h. Ethyl acetate (20 mL) is added and the solution is washed with 1N sodium hydroxide (3×10 mL), water (10 mL), 0.1N hydrochloric acid (3×10 mL) and brine (10 mL). The organic layer is dried over magnesium sulfate, filtered and evaporated to give a yellow solid. Yield 0.201 g (68%). ESI-MS (m/z): 396[M−H]⁻. The product is dissolved in ethanol (3 mL) and degassed with argon for 2 min. 10% Palladium on carbon (0.08 g) is then added and hydrogen is bubbled through the stirring solution for 3 h. The reaction mixture is filtered through celite and the solvent is evaporated to give a yellow solid. Yield 0.175 g (95%). ESI-MS (m/z): 366[M−H]⁻. The product is dissolved in pyridine (3 mL) and sulfur trioxide pyridine complex (0.227 g, 1.2 mmol) is added to the stirring solution. The resulting heterogeneous solution is stirred for 5 min then quenched with 7% ammonium hydroxide (aq) (25 mL) and the solution is stirred for an additional 5 min. The solvents are evaporated to dryness and the residue is re-dissolved in 7% ammonium hydroxide (aq) (25 mL) and evaporated to dryness. The resulting solid is HPLC purified to give 0.103 g (46%) (30% over 3 steps) of an off-white solid.

Mixture of rotational isomers. ¹H NMR (D₂O) δ 7.28-6.88 (m, 7H), 4.60-4.32 (m, 3H), 3.78 (s, 3H), 3.13-2.79 (m, 6H), 2.58 (d, 3H). ¹³C {¹H} NMR (D₂O) δ 176.34, 174.08, 173.76, 157.70, 157.60, 139.23, 139.04, 134.15, 133.82, 133.20, 129.95, 128.60, 127.86, 127.65, 118.68, 117.40, 117.05, 114.51, 114.39, 112.54, 57.49, 55.69, 55.60, 55.39, 46.61, 44.43, 35.58, 35.16, 31.46, 30.76, 30.49, 26.20, 26.10. ESI-MS (m/z): 446[M−H]⁻. Anal. Calcd. for C₂₁H₂₈N₄O₆S.1/3H₂O: C, 53.60; H, 6.14; N, 11.91. Found: C, 53.84; H, 6.24; N, 11.91.

### Example 12

### (S)-[3-Benzylcarbamoyl-2-(4-propyl-benzoyl)-1,2,3,4-tetrahydro-isoquinolin-7-yl]-sulfamic Acid

(S)-7-Nitro-3,4-dihydro-1H-isoquinoline-2,3-dicarboxylic acid 2-tert-butyl ester (5.0g, 15.5 mmol) is dissolved in dichloromethane (20 mL) and cooled to 0C. EDC (3.58 g, 18.6 mmol) is added and the reaction is stirred for 30 min. Benzylamine (2.03 mL, 18.6 mmol) is added slowly and reaction is allowed to warm slowly to room temperature over a period of 18 h. The solvent is evaporated and the brown oil is purified by flash column chromatography (19:1 chloroform:methanol) to give an orange solid. Yield 4.73 g (75%). ESI-MS (m/z): 334[M−H]⁻.

### (S)-7-Nitro-1,2,3,4-tetrahydro-isoquinoline-3-carboxylic Acid Benzylamide Triflouroacetate

(S)-3-Methylcarbamoyl-7-nitro-3,4-dihydro-1H-isoquinoline-2-carboxylic acid tert-butyl ester (2.5 g, 6.1 mmol) is dissolved in 95% TFA/H₂O (20 mL) and stirred for 1.5 h. The solvent is evaporated to dryness to give a brown orange powder. Yield 1.68 g (65%). ¹H NMR (CDCl₃) δ 8.30-8.18 (m, 5H), 7.60 (m, 2H), 7.35 (m, 1H), 4.40 (m, 3H), 3.60 (m, 2H), 3.38 (m, 2H). ¹³C {¹H} NMR (CDCl₃) δ 170.0, 149.0, 139.8, 131.9, 131.4, 130.1, 129.2, 129.0, 124.3, 123.5, 56.2, 45.5, 44.8, 31.3. ESI-MS (m/z): 312[M+H]⁺.

### (S)-[3-Benzylcarbamoyl-2-(4-propyl-benzoyl)-1,2,3,4-tetrahydro-isoquinolin-7-yl]-sulfamic Acid

(S)-7-Nitro-1,2,3,4-tetrahydro-isoquinoline-3-carboxylic acid benzylamide triflouroacetate (0.20 g, 0.47 mmol) is slurried in dichloromethane (3 mL) and diisopropylethylamine (0.168 mL, 1.0 mmol) is added to the stirring solution. 4-Propylbenzoyl chloride (0.129 mL, 0.7 mmol) is added and the reaction is stirred for 2 h. Ethyl acetate (20 mL) is added and the solution is washed with 1N sodium hydroxide (3×10 mL) and brine (10 mL). The organic layer is dried over magnesium sulfate, filtered and evaporated to give a yellow solid. Yield 0.192 g (89%). ESI-MS (m/z): 456[M−H]⁻. The product is dissolved in ethanol (3 mL) and degassed with argon for 2 min. 10% Palladium on carbon (0.08 g) is added and hydrogen is bubbled through the stirring solution for 3 h. The reaction mixture is filtered through celite and the solvent is evaporated to give a yellow solid. Yield 0.132 g (78%) ESI-MS (m/z): 426[M−H]⁻. The product is dissolved in pyridine (3 mL) and sulfur trioxide pyridine complex (0.147 g, 0.9 mmol) is added to the stirring solution. The resulting heterogeneous solution is stirred for 5 min. then quenched with 7% ammonium hydroxide (aq) (25 mL) and the solution is stirred for an additional 5 min. The solvents are evaporated to dryness and the residue is re-dissolved in 7% ammonium hydroxide (aq) (25 mL) and evaporated to dryness. The resulting solid is HPLC purified to give 0.097 g (60%) (29% over 3 steps) of an off-white solid.

Mixture of rotational isomers. ¹HNMR (D₂O) δ 7.33-7.10 (m, 5H), 6.99-6.85 (m, 3H), 6.80-6.50 (m, 4H), 4.50-4.35 (m, 2H), 4.28-3.85 (m, 3H), 3.20-2.85 (m, 2H), 2.58-2.39 (m, 2H), 1.59-1.48 (m, 2H), 0.85-0.67 (m, 3H). ¹³C {(H} NMR (D₂O) δ 174.63, 173.93, 173.66, 172.71, 146.51, 145.81, 139.47, 139.23, 137.96, 137.68, 134.53, 133.25, 132.45, 131.90, 129.10, 128.92, 128.79, 128.58, 128.23, 127.39, 126.88, 126.74, 126.26, 118.73, 118.25, 117.16, 116.90, 59.02, 56.84, 49.10, 45.20, 42.83, 37.40, 37.31, 31.78, 30.99, 24.14, 24.05, 13.24. ESI-MS (m/z): 506[M−H]⁻. Anal. Calcd. for C₂₇H₃₂N₄O₅S.1 1/2H₂O: C, 58.78; H, 6.39; N, 10.16. Found: C, 58.48; H, 6.04; N, 9.99.

### Example 13

### (S)-[3-Benzylcarbamoyl-2-(3-phenyl-propionyl)-1,2,3,4-tetrahydro-isoquinolin-7yl]-sulfamic Acid

(S)-7-Nitro-1,2,3,4-tetrahydro-isoquinoline-3-carboxylic acid benzylamide trifluoroacetate (0.20 g, 0.47 mmol) is slurried in dichloromethane (3 mL) and diisopropylethylamine (0.168 mL, 1.0 mmol) is added to the stirring solution. Hydrocinnamoyl chloride (0.067 mL, 0.7 mmol) is added and the reaction is stirred for 2 h. Ethyl acetate (20 mL) is added and the solution is washed with 1N sodium hydroxide (3×10 mL) and brine (10 mL). The organic layer is dried over magnesium sulfate, filtered and evaporated to give a yellow solid. Yield 0.130 g (62%). ESI-MS (m/z): 442[M−H]⁻. The product is dissolved in ethanol (3 mL) and degassed with argon for 2 min. 10% Palladium on carbon (0.08 g) is then added and hydrogen is bubbled through the stirring solution for 3 h. The reaction mixture is filtered through celite and the solvent is evaporated to give a yellow solid. Yield 0.084 g (70%). ESI-MS (m/z): 412[M−H]⁻. The product is dissolved in pyridine (3 mL) and sulfur trioxide pyridine complex (0.098 g, 0.6 mmol) is added to the stirring solution. The resulting heterogeneous solution is stirred for 5 min then quenched with 7% ammonium hydroxide (aq) (25 mL) and the solution is stirred for an additional 5 min. The solvents are evaporated to dryness and the residue is re-dissolved in 7% ammonium hydroxide (aq) (25 mL) and evaporated to dryness. The resulting solid is HPLC purified to give 0.041 g (40%) (17% over 3 steps) of an off-white solid.

Mixture of rotational isomers. ¹H NMR (D₂O) δ 7.22-7.03 (m, 8H), 6.95 (m, 3H), 6.52-6.42 (m, 2H), 4.594.12 (m, 4H), 3.90-3.82 (m, 1H), 3.05-2.79 (m, 6H). ¹³C {¹H} NMR (D₂O) δ 176.57, 176.13, 173.64, 173.22, 140.80, 140.69, 139.32, 137.67, 137.56, 133.76, 129.02, 128.94, 128.79, 128.66, 128.58, 127.93, 127.23, 126.78, 126.38, 126.30, 118.61, 118.41, 117.30, 117.09, 57.52, 55.91, 46.73, 44.88, 42.91, 42.61, 35.44, 34.98, 31.61, 31.13, 30.98, 30.88. ESI-MS (m/z): 492[M−H]⁻. Anal. Calcd. for C₂₆H₃₀N₄O₅S.2H₂O: C, 57.13; H, 6.27; N, 10.25. Found: C, 57.33; H, 5.62; N, 10.20.

### Example 14

### (S)-3-Benzylcarbamoyl-7-sulfoamino-3,4-dihydro-1H-isoquinoline-2-carboxylic Acid Benzyl Ester

(S)-7-Nitro-1,2,3,4-tetrahydro-isoquinoline-3-carboxylic acid benzylamide trifluoroacetate (0.20 g, 0.47 mmol) is dissolved in dioxane(3 mL) and a solution of sodium carbonate (0.136 g, 1.3 mmol) in water (2 mL) is added. The mixture is cooled to 0° C. Benzylchloroformate (0.087 mL, 0.6 mmol) is added and the reaction mixture is taken out of the cooling bath and stirred for 1 h. Ethyl acetate is added (50 mL) and the organic layer is washed with water (3×25 mL). The organic layer is dried over magnesium sulfate, filtered and evaporated to give a white solid. Yield 0.208 g (99%). ESI-MS (m/z): 444[M−H]⁻. The product is dissolved in 1:1 ethyl acetate/ethanol (5 mL) and tin(II) chloride dihydrate (0.551 g, 2.4 mmol) is added and the reaction mixture is heated at 50° C. for 3 h. Ethyl acetate (30 mL) is added and the solution is washed with 1N sodium hydroxide (3×20 mL). The organic layer is dried over magnesium sulfate, filtered and evaporated to give a yellow solid. Yield 0.180 g (94%). ESI-MS (m/z): 414[M−H]⁻. The product is dissolved in pyridine (3 mL) and sulfur trioxide pyridine complex (0.217 g, 1.4 mmol) is added to the stirring solution. The resulting heterogeneous solution is stirred for 5 min then quenched with 7% ammonium hydroxide (aq) (25 mL) and the solution is stirred for an additional 5 min. The solvents are evaporated to dryness and the residue is re-dissolved in 7% ammonium hydroxide (aq) (25 mL) and evaporated to dryness. The resulting solid is HPLC purified to give 0.085 g (39%) (35% over 3 steps) of an off-white solid.

Mixture of rotational isomers. ¹H NMR (D₂O) δ 7.58-7.03 (m, 11H), 6.70 (m, 2H), 5.30-5.06 (m, 2H), 4.69 (m, 2H), 4.55-3.92 (m, 3H), 3.10 (s, 2H). ¹³C {(H} NMR (D₂O) δ 174.58, 174.26, 157.75, 157.17, 139.47, 137.91, 136.40, 136.28, 134.72, 134.39, 129.15, 129.04, 128.92, 128.71, 128.36, 128.05, 127.77, 127.42, 126.73, 126.43, 118.72, 118.60, 117.39, 68.45, 68.24, 56.90, 56.79, 45.37, 42.80, 31.50, 31.33. ESI-MS (m/z): 494[M−H]⁻. Anal. Calcd. for C₂₅H₂₈N₄O₆S.H₂O: C, 56.59; H, 5.70; N, 10.56. Found: C, 56.63; H, 5.32; N, 10.45.

### Example 15

### (S)-[3-Methylcarbamoyl-2-[3-(3-sulfamoyl-Phenyl)-propionyl]-1,2,3,4-tetrahydro-isoquinolin-7-yl]-sulfamic Acid

3-(3-Sulfamoyl-phenyl)-propionic acid (0.300 g, 1.31 mmol) and N-hydroxybenzotriazole (0.219 g, 1.43 mmol) is dissolved in DMF (1 mL). To this solution is added dichloromethane (1 mL) and the reaction is cooled to 0° C. EDC (0.274, 1.43 mmol) is added and the reaction is stirred for 30 min. In a separate flask (S)-3-methylcarbamoyl-7-nitro-1,2,3,4-tetrahydro-isoquinoline hydrochloride (0.534 g, 1.96 mmol) is dissolved in dichloromethane (1 mL) and N,N-disopropylethyl amine (0.253 g, 1.96 mmol). This solution is added to the cooling solution slowly over several minutes and the reaction is stirred for 72 h. The reaction is concentrated to yield a viscous oil which is diluted with dichloromethane (25 muL) and is washed with 1.0 N hydrochloric acid (3×25 mL) and brine (25 mL). The organic layer is dried over magnesium sulfate, filtered and evaporated to give a yellow solid. The crude material is purified by flash column chromatography on silica gel eluting with 7.5:1 chloroform/methanol. Yield 0.485 g (83%). ESI-MS (m/z): 447[M+H]⁺. The product (0.170 g, 0.381 mmol) is dissolved in methanol (6 mL) and degassed with argon for 2 min. 10% Palladium on carbon (0.034 g) is added and hydrogen is bubbled through the stirring solution for 5 h. The reaction mixture is filtered through celite and the solvent is evaporated to give a yellow solid. ESI-MS (m/z): 417[M+H]⁺. The product is dissolved in pyridine (2 mL) and sulfur trioxide pyridine complex (0.151 g, 0.95 mmol) is added to the stirring solution. The resulting heterogeneous solution is stirred for 5 min then quenched with 7% ammonium hydroxide (aq) (15 mL) and the solution is stirred for an additional 5 min. The solvents are evaporated to dryness and the residue is re-dissolved in 7% ammonium hydroxide (aq) (25 mL) and evaporated to dryness. The resulting solid is HPLC purified to give 0.05 g (27%-2 steps) of an off-white solid.

Mixture of rotational isomers. ¹H NMR (D₂O) δ 7.64-7.54 (m, 2H), 7.44-7.33 (m, 2H), 7.02-6.81 (m, 3H), 4.65-4.27 (m, 3H), 2.98-2.68 (m, 6H), 2.41 (s, 2H), 2.36 (s, 1H). ESI-MS (m/z): 495[M−H]⁻. Anal. Calcd. for C₂₀H₂₇N₅O₇S₂: C, 46.77; H, 5.30; N, 13.64. Found: C, 46.51; H, 5.28; N, 13.78.

### Example 16

### (S)-{2-[3-(3-Acetylsulfamoyl-phenyl)-propionyl]-3-methylcarbamoyl-1,2,3,4-tetrahydro-isoquinolin-7-yl}-sulfamic Acid

3-(3-Acetylsulfamoyl-phenyl)-propionic acid (0.148 g, 0.55 mmol) and N-hydroxybenzotriazole (0.084 g, 0.55 mmol) are dissolved in DMF (1 mL). To this solution is added dichloromethane (1.5 mL) and the reaction is cooled to 0° C. EDC (0.105, 0.55 mmol) is added and the reaction is stirred for 30 min. In a separate flask (S)-3-methylcarbamoyl-7-nitro-1,2,3,4-tetrahydro-isoquinoline hydrochloride (0.223 g, 0.82 mmol) is dissolved in dichloromethane (1 mL) and N,N-disopropylethyl amine (0.106 g, 0.82 mmol). This solution is added to the cooling solution slowly over several minutes and the reaction is stirred for 72 h. The reaction is concentrated to yield a viscous oil which is diluted with dichloromethane (25 mL) and washed with 1.0 N hydrochloric acid (2×25 mL) and brine (25 mL). The organic layer is dried over magnesium sulfate, filtered and evaporated to give a yellow solid. The crude material is purified by flash column chromatography on silica gel eluting with 10:1 chloroform/methanol. Yield 0.177 g (66%). ESI-MS (m/z): 489[M+H]⁺. The product (0.150 g, 0.31 mmol) is dissolved in methanol (6 mL) and degassed with argon for 2 min. 10% Palladium on carbon (0.06 g) is added and hydrogen is bubbled through the stirring solution for 5 h. The reaction mixture is filtered through celite and the solvent is evaporated to give a yellow solid. The product is dissolved in pyridine (3 mL) and sulfur trioxide pyridine complex (0.150 g, 0.93 mmol) is added to the stirring solution. The resulting heterogeneous solution is stirred for 5 min then quenched with 7% ammonium hydroxide (aq) (15 mL) and the solution is stirred for an additional 5 min. The solvents are evaporated to dryness and the residue is re-dissolved in 7% ammonium hydroxide (aq) (25 mL) and evaporated to dryness. The resulting solid is HPLC purified to give 0.06 g (35%-2 steps) of an off-white solid.

Mixture of rotational isomers. ¹H NMR (D₂O) δ 7.63-7.56 (m, 2H), 7.39-7.32 (m, 2H), 7.04-6.88 (m, 3H), 4.56-4.25 (m, 3H), 2.99-2.40 (m, 6H), 2.39 (s, 2H), 2.37 (s, 1H), 1.77 (d, 3H). ¹³C{¹H} NMR (D₂O) δ 179.25, 179.02, 176.07, 175.99, 174.58, 173.91, 142.56, 142.31, 141.67, 141.43, 139.74, 139.59, 134.48, 133.72, 133.62, 130.01, 129.91, 128.99, 128.68, 127.89, 127.09, 125.53, 125.42, 120.15, 119.36, 119.13, 117.95, 117.77, 57.77, 56.08, 49.68, 47.05, 44.92, 35.31, 35.07, 31.93, 31.55, 31.13, 26.59, 26.45, 25.24, 25.17. ESI-MS (m/z): 537[M−H]⁻. Anal. Calcd. for C₂₂H₃₂N₆08S₂3/4H₂O: C, 45.08; H, 5.76; N, 14.34. Found: C, 44.82; H, 5.51; N, 14.61.

### Example 17

### (S)-{3-Methylcarbamoyl-2-[3-(3-propionylsulfamoyl-phenyl)-propionyl]-1,2,3,4-tetrahydro isoquinolin-7-yl}-sulfamic Acid

(S)-7-Nitro-2-[3-(3-sulfamoyl-phenyl)-propionyl]-1,2,3,4-tetrahydro-isoquinoline-3-carboxylic acid methylamide (0.091 g, 0.2 mmol) are dissolved in acetonitrile (1.5 mL) and propionic acid anhydride (0.032 mL, 0.24 mmol) is added followed by DMAP (0.024 g, 0.2 mmol). The reaction is allowed to stir for 18 h. at which point additional propionic acid anhydride (0.032 mL, 0.2 mmol) is added and the reaction is continued for 2.5 h. The reaction is diluted with ethyl acetate (25 mL) and the organic layer is washed with 0.1 N HCl (2×25 mL) followed by brine (1×25 mL) and dried over sodium sulfate. Concentration yields an off-white solid. Yield 0.1 g (100%) MS m/z 501[M−H]⁻.

(S)-7-Nitro-2-[3-(3-propionylsulfamoyl-phenyl)-propionyl]-1,2,3,4-tetrahydroisoquinoline-3-carbixylic acid methylamide (0.09 g, 0.18 mmol) is dissolved in methanol (4 mL) and degassed with argon for several minutes. 10% Palladium on carbon (0.06 g) is added and hydrogen is introduced into the flask via balloon. The reaction is agitated for 2 h before filtering through celite and concentrating to give an oily brown residue. MS m/z 473[M+H]⁺.

(S)-7-Amino-2-[3-(3-propionylsulfamoyl-phenyl)-propionyl]-1,2,3,4-tetrahydroisoquinoline-3-carbixylic acid methylamide (0.18 mmol) is dissolved in pyridine (3 mL) and sulfur trioxide pyridine complex (0.086 g, 0.54 mmol) is added in one portion. The reaction is allowed to stir for 4 min then quenched with 7% ammonium hydroxide (aq) (20 mL). The reaction is concentrated and purified by RP-HPLC. Yield 0.057 g (56%).

Mixture of rotational isomers: ¹H NMR (D₂O) δ 7.6 (m, 2H), 7.3 (m, 2H), 6.9 (m, 3H), 4.6-4.2 (m, 3H), 3.0-2.4 (m, 6H), 2.4 (d, 3H), 2.0 (qt, 2H), 0.9 (t, 3H). MS m/z 551[M−H]⁻. Anal. Calcd. for C₂₃H₂₈N₄O₈S₂.1.5 NH₃.0.5H₂O: C, 47.05; H, 5.75; N, 13.12. Found: C, 46.82; H, 5.56; N, 12.89.

### Example 18

### (S)-(2-{3-[3-(2,2-Dimethyl-propionylsulfamoyl)-phenyl]-propionyl}-3-methylcarbamoyl 1,2,3,4-tetrahydro-isoquinolin-7-yl)-sulfamic Acid

(S)-7-Nitro-2-[3-(3-sulfamoyl-phenyl)-propionyl]-1,2,3,4-tetrahydro-isoquinoline-3-carboxylic acid methylamide (0.09 g, 0.2 mmol) are dissolved in acetonitrile (3 mL) and trimethylacetic acid anhydride (0.049 mL, 0.24 mmol) is added followed by DMAP (0.024 g, 0.2 mmol). The reaction is allowed to stir for 18 h. at which point additional trimethylacetic acid anhydride (0.05 mL, 0.2 mmol) is added and the reaction is heated to 65° C. for 24 h. The reaction is diluted with ethyl acetate (25 mL) and the organic layer is washed with 0.1 N HCl (2×25 mL) followed by brine (1×25 mL) and dried over sodium sulfate. The crude material is purified by flash column chromatography on silica gel eluting with 9:1 chloroform/methanol. Concentration yields a slightly yellow solid. Yield 0.123 g MS m/z 531[M+H]⁺.

(S)-2-{3-[3-(2,2-Dimethyl-propionylsulfamoyl)-phenyl]-propionyl}-7-nitro-1,2,3,4-tetrahydro-isoquinoline-3-carboxylic acid methylamide (0.1 g, 0.19 mmol) is dissolved in methanol (6 mL) and degassed with argon for several minutes. 10% Palladium on carbon (0.06 g) is added and hydrogen is introduced into the flask via balloon. The reaction is agitated for 2 h before filtering through celite and concentrating to give an oily brown residue. MS m/z 501[M+H]⁺.

(S)-2-{3-[3-(2,2-Dimethyl-propionylsulfamoyl)-phenyl]-propionyl}-7-amino-1,2,3,4-tetrahydro-isoquinoline-3-carboxylic acid methylamide (0.18 mmol) is dissolved in pyridine (3 mL) and sulfur trioxide pyridine complex (0.09 g, 0.57 mmol) is added in one portion. The reaction is allowed to stir for 4 min then quenched with 7% ammonium hydroxide (aq) (20 mL). The reaction is concentrated and purified by RP-HPLC. Yield 0.015 g (13%).

Mixture of rotational isomers: ¹H NMR (D₂O) δ 7.6 (m, 2H), 7.3 (m, 2H), 6.9 (m, 3H), 4.6-4.2 (m, 3H), 3.0-2.4 (m, 6H), 2.4 (d, 3H), 0.9 (s, 9H). MS m/z 579[M−H]⁻. Anal. Calcd. for C₂₅H₃₂N₄O₈S₂.1.25 NH₃.2.0H₂O: C, 47.06; H, 6.28; N, 11.53. Found: C, 46.91; H, 6.28; N, 11.59.

### Example 19

### (S)-{2-[3-(3-Benzoylsulfamoyl-phenyl)-propionyl]-3-methylcarbamoyl-1,2,3,4-tetrahydro-isoquinolin-7-yl}-sulfamic Acid

(S)-7-Nitro-2-[3-(3-sulfamoyl-phenyl)-propionyl]-1,2,3,4-tetrahydro-isoquinoline-3-carboxylic acid methylamide (0.276 g, 0.62 mmol) is dissolved in acetonitrile (5 mL) and diisopropylethyl amine (0.150 mL, 0.86 mmol) is added followed by benzoyl chloride (0.086 mL, 0.74 mmol) and DMAP (0.076 g, 0.62 mmol). The reaction is allowed to stir for 3 h. then concentrated to give a yellow-orange residue. The crude material is purified by flash column chromatography on silica gel eluting with 9:1 chloroform/methanol. Concentration yields a slightly yellow solid. Yield 0.283 g (82%) MS m/z 551[M+H]⁺.

(S)-2-[3-(3-Benzoylsulfamoyl-phenyl)-propionyl]-7-nitro-1,2,3,4-tetrahydro-isoquinoline-3-carboxylic acid methylamide (0.283 g, 0.51 mmol) is dissolved in methanol (10 mL) and degassed with argon for several minutes. 10% Palladium on carbon (0.100 g) is added and hydrogen is introduced into the flask via balloon. The reaction is agitated for 18 h before filtering through celite and concentrating to give an oily brown residue. MS m/z 521[M+H]⁺.

(S)-2-[3-(3-Benzoylsulfamoyl-phenyl)-propionyl]-7-amino-1,2,3,4-tetrahydro-isoquinoline-3-carboxylic acid methylamide (0.51 mmol) is dissolved in pyridine (5 mL) and sulfur trioxide pyridine complex (0.243 g, 1.53 mmol) is added in one portion. The reaction is allowed to stir for 4 min then quenched with 7% ammonium hydroxide (aq) (25 mL). The reaction is concentrated and purified by RP-HPLC. Yield 0.159 g (50%).

Mixture of rotational isomers: ¹H NMR (D₂O) δ 7.8-7.6 (m, 4H), 7.5-7.2 (m, 5H), 7.0-6.6 (m, 3H), 4.6-4.3 (m, 2H), 4.1 (dd, 1H), 3.0-2.6 (m, 6H), 2.3 (d, 3H). MS m/z 599[M−H]⁻. Anal. Calcd. for C₂₇H₂₈N₄O₈S₂.1.75 NH₃.1.0H₂O: C, 50.01; H, 5.48; N, 12.42. Found: C, 50.25; H, 5.56; N, 12.58.

### Example 20

### (S)-{3-Methylcarbamoyl-2-[3-(4-sulfamoyl-phenyl)-propionyl]-1,2,3,4-tetrahydro-isoquinolin-7-yl}-sulfamic Acid

3-(4-Sulfamoyl-phenyl)-propionic acid (0.250 g, 1.09 mmol) and N-hydroxybenzotriazole (0.184 g, 1.31 mmol) is dissolved in DMF (1 mL). To this solution is added dichloromethane (1 mL) and the reaction is cooled to 0° C. EDC (0.208, 1.09 mmol) is added and the reaction is stirred for 30 min. In a separate flask (S)-3-methylcarbamoyl-7-nitro-1,2,3,4-tetrahydro-isoquinoline hydrochloride (0.355 g, 1.31 mmol) is dissolved in dichloromethane (1 mL) and N,N-disopropylethyl amine (0.253 g, 1.96 mmol). This solution is added to the cooling solution slowly over several minutes and the reaction is stirred for 72 h. The reaction is concentrated to yield a viscous oil which is diluted with dichloromethane (25 mL) and is washed with 1.0 N hydrochloric acid (3×25 mL), sodium carbonate (sat. aq.) (2×25) and brine (25 mL). The organic layer is dried over sodium sulfate, filtered and evaporated to give a yellow solid. The crude material is purified by flash column chromatography on silica gel eluting with 7.5:1 chloroform/methanol. Yield 0.296 g (61%). ESI-MS (m/z): 447[M+H]⁺. The product (0.094 g, 0.21 mmol) is dissolved in methanol (5 mL) and degassed with argon for 2 min. 10% Palladium on carbon (0.08 g) is added and hydrogen is bubbled through the stirring solution for 2 h. The reaction mixture is filtered through celite and the solvent is evaporated to give a yellow solid. ESI-MS (m/z): 417[M+H]⁺. The product is dissolved in pyridine (3 mL) and sulfur trioxide pyridine complex (0.100 g, 0.63 mmol) is added to the stirring solution. The resulting heterogeneous solution is stirred for 5 min then quenched with 7% ammonium hydroxide (aq) (15 mL) and the solution is stirred for an additional 5 min. The solvents are evaporated to dryness and the residue is re-dissolved in 7% ammonium hydroxide (aq) (25 mL) and evaporated to dryness. The resulting solid is HPLC purified to give 0.056 g (53%-2 steps) of an off-white solid. Mixture of rotational isomers. ¹H NMR (D₂O) δ 7.72 (d, 2H), 7.39 (d, 1.5H), 7.35 (d, 0.5H), 7.1-6.9 (m, 3H), 4.6-4.4 (m, 3H), 3.1-2.9 (m, 4H), 2.9-2.5 (m, 2H), 2.4 (d, 3H), ESI-MS (m/z): 495[M−H]⁻. Anal. Calcd. for C₂₀H₂₄N₄O₇S₂.1.0 NH₃ 1.0H₂O: C, 45.19; H, 5.50; N, 13.17. Found: C, 45.24; H, 5.45; N, 13.33.

### Example 21

### (S)-{2-[3-(4-Acetylsulfamoyl-phenyl)-propionyl]-3-methylcarbamoyl-1,2,3,4-tetrahydro-isoquinolin-7-yl}-sulfamic Acid

(S)-7-Nitro-2-[3-(4-sulfamoyl-phenyl)-propionyl]-1,2,3,4-tetrahydro-isoquinoline-3-carboxylic acid methylamide (0.105 g, 0.24 mmol) is dissolved in acetonitrile (5 mL) and acetic anhydride (0.027 mL, 0.28 mmol) is added followed by DMAP (0.029 g, 0.24 mmol). The reaction is allowed to stir for 18 h. The volatiles are removed and the crude material is purified by flash column chromatography eluting with 9:1 chloroform/methanol to give a light yellow solid. Yield 0.1 g (85%) MS m/z 487[M−H]⁻.

The crude material (0.1 g, 0.2 mmol) is dissolved in methanol (5 mL) and degassed with argon for several minutes. 10% Palladium on carbon (0.05 g) is added and hydrogen is introduced into the flask via balloon. The reaction is agitated for 1.5 h before filtering through celite and concentrating to give an oily residue. MS m/z 459[M+H]⁺.

The crude product (0.2 mmol) is dissolved in pyridine (3 mL) and sulfur trioxide pyridine complex (0.098 g, 0.62 mmol) is added in one portion. The reaction is allowed to stir for 4 min then quenched with 7% ammonium hydroxide (aq) (20 mL). The reaction is concentrated and purified by RP-HPLC. Yield 0.062 g (56%).

Mixture of rotational isomers: ¹H NMR (D₂O) δ 7.72 (d, 2H), 7.39 (m, 2H), 7.1-6.9 (m, 3H), 4.6-4.4 (m, 3H), 3.1-2.5 (m, 6H), 2.4 (d, 3H) 1.8 (d, 3H), MS m/z 537[M−H]⁻. Anal. Calcd. for C₂₂H₂₆N₄O₈S₂.1.5 NH₃.0.5H₂O: C, 46.10; H, 5.54; N, 13.44. Found: C, 45.81; H, 5.55; N, 13.53.

### Example 22

### (R)-{3-Methylcarbamoyl-2-[3-(3-sulfamoyl-phenyl)-propionyl]-1,2,3,4-tetrahydro-isoquinolin-7-yl}-sulfamic Acid

3-(3-Sulfamoyl-phenyl)-propionic acid (0.203 g, 0.884 mmol) and N-hydroxybenzotriazole (0.162 g, 1.06 mmol) is dissolved in DMF (1 mL). To this solution is added dichloromethane (1 mL) and the reaction is cooled to 0° C. EDC (0.186, 0.973 mmol) is added and the reaction is stirred for 30 min. In a separate flask (R)-3-methylcarbamoyl-7-nitro-1,2,3,4-tetrahydro-isoquinoline hydrochloride (0.264 g, 0.973 mmol) is dissolved in dimethylformamide (1 mL) and N,N-disopropylethyl amine (0.307 mL, 1.77 mmol). This solution is added to the cooling solution slowly over several minutes and the reaction is stirred for 72 h. The reaction is diluted with ethyl acetate (25 mL) and is washed with 1.0 N hydrochloric acid (3×25 mL), sodium carbonate (sat. aq.) (2×25 mL) and brine (25 mL). The organic layer is dried over sodium sulfate, filtered and evaporated to give a yellow solid. The crude material is purified by flash column chromatography on silica gel eluting with 7.5:1 chloroform/methanol. Yield 0.273 g (69%). ESI-MS (m/z): 447[M+H]⁺. The product (0.104 g, 0.381 mmol) is dissolved in methanol (3 mL) and degassed with argon for 2 min. 10% Palladium on carbon (0.05 g) is added and hydrogen is bubbled through the stirring solution for 1 h. The reaction mixture is filtered through celite and the solvent is evaporated to give a yellow solid. ESI-MS (m/z): 417[M+H]⁺. The product is dissolved in pyridine (3 mL) and sulfur trioxide pyridine complex (0.111 g, 0.7 mmol) is added to the stirring solution. The resulting heterogeneous solution is stirred for 5 min then quenched with 7% ammonium hydroxide (aq) (15 mL) and the solution is stirred for an additional 5 min. The solvents are evaporated to dryness and the residue is re-dissolved in 7% ammonium hydroxide (aq) (25 mL) and evaporated to dryness. The resulting solid is HPLC purified to give 0.033 g (28%-2 steps) of an off-white solid.

Mixture of rotational isomers. ¹H NMR (D₂O) δ 7.7-7.5 (m, 2H), 7.5-7.3 (m, 2H), 7.0-6.8 (m, 3H), 4.5-4.2 (m, 3H), 3.0-2.4 (m, 6H), 2.41 (d, 3H). ESI-MS (m/z): 495[M−H]⁻. Anal. Calcd. for C₂₀H₂₄N₄O₇S₂.0.8 NH₃.1.0H₂O: C, 45.48; H, 5.42; N, 12.73. Found: C, 45.20; H, 5.20; N, 12.76.

### Example 23

### (R)-{2-[3-(3-Acetylsulfamoyl-phenyl)-propionyl]-3-methylcarbamoyl-1,2,3,4-tetrahydro-isoquinolin-7-yl}-sulfamic Acid

(R)-7-Nitro-2-[3-(3-sulfamoyl-phenyl)-propionyl]-1,2,3,4-tetrahydro-isoquinoline-3-carboxylic acid methylamide (0.158 g, 0.35 mmol) is dissolved in acetonitrile (3 mL) and acetic anhydride (0.040 mL, 0.42 mmol) is added followed by DMAP (0.042 g, 0.35 mmol). The reaction is allowed to stir for 18 h. The reaction is diluted with ethyl acetate (40 mL) and washed with 0.1 N HCl (3×25 mL) then brine (1×25 mL) and dried over sodium sulfate. After concentration the crude material was purified by flash column chromatography eluting with 9:1 chloroform/methanol to give a light yellow solid. Yield 0.123 g (72%) MS m/z 489[M+H]⁺.

The crude material (0.123 g, 0.25 mmol) is dissolved in methanol (4.5 mL) and degassed with argon for several minutes. 10% Palladium on carbon (0.075 g) is added and hydrogen is introduced into the flask via balloon. The reaction is agitated for 1 h before filtering through celite and concentrating to give an oily residue. MS m/z 459[M+H]⁺.

The crude product (0.25 mmol) is dissolved in pyridine (31 mL) and sulfur trioxide pyridine complex (0.120 g, 0.76 mmol) is added in one portion. The reaction is allowed to stir for 4 min then quenched with 7% ammonium hydroxide (aq) (20 mL). The reaction is concentrated and purified by RP-HPLC. Yield 0.091 g (66%).

Mixture of rotational isomers. ¹H NMR (D₂O) δ 7.7-7.5 (m, 2H), 7.5-7.2 (m, 2H), 7.0-6.8 (m, 3H), 4.6-4.2 (m, 3H), 3.0-2.4 (m, 6H), 2.4 (d, 3H), 1.8 (d, 3H). ESI-MS (m/z): 537[M−H]⁻. Anal. Calcd. for C₂₂H₂₆N₄O₈S₂.1.5 NH₃: C, 46.84; H, 5.45; N, 13.66. Found: C, 46.69; H, 5.37; N, 13.66.

### Example 24

### (S)-3-[3-(3-Methylcarbamoyl-7-sulfoamino-3,4-dihydro-1H-isoquinolin-2-yl)-3-oxo-propyl]-benzoic Acid

3-(2-Carboxy-ethyl)-benzoic acid methyl ester (0.150 g, 0.72 mmol) and N-hydroxybenzotriazole (0.132 g, 0.86 mmol) are dissolved in DMF (1 mL). To this solution is added dichloromethane (1.0 mL) and the reaction is cooled to 0° C. EDC (0.166, 0.86 mmol) is added and the reaction is stirred for 30 min. In a separate flask (S)-3-methylcarbamoyl-7-nitro-1,2,3,4-tetrahydro-isoquinoline hydrochloride (0.216 g, 0.8 mmol) is dissolved in dichloromethane (1 mL) and N,N-disopropylethyl amine (0.14 g, 0.1.1 mmol). This solution is added to the cooling solution slowly over several minutes and the reaction is stirred for 3 h. The reaction is diluted with ethyl acetate (25 mL) and washed with 1.0 N hydrochloric acid (2×25 mL), sodium carbonate (sat. aq) (2×25 mL) and brine (25 mL). The organic layer is dried over sodium sulfate, filtered and evaporated to give a yellow solid. The crude material is purified by flash column chromatography on silica gel eluting with 30:1 chloroform/methanol. Yield 0.192 g (63%). ESI-MS (m/z): 426[M+H]⁺. The product (0.191 g, 0.45 mmol) is dissolved in methanol and a solution of lithium hydroxide (0.042 g, 0.9 mmol) in water (1 mL) is added. The reaction is stirred for 4 h. and additional lithium hydroxide (0.02 g, 0.45 mmol) in water (0.5 mL) is added. The reaction is allowed to stir for an additional 2 h at which point the volatiles are removed and 1 N HCl is added to the resulting residue. The acidic solution is extracted with ethyl acetate (3×25 mL). The organic fractions are pooled and washed with brine (2×25 mL), dried over sodium sulfate and concentrated to give and orange oily solid. The crude material is purified by RP-HPLC to give an orange solid. Yield 0.112 g (61%). MS m/z 410[M−H]⁻. The product (0.110 g, 0.27 mmol) is dissolved in THF (5 mL) and degassed with argon for 2 min. 10% Palladium on carbon (0.05 g) is added and hydrogen is bubbled through the stirring solution for 4.5 h. The reaction mixture is filtered through celite and the solvent is evaporated to give a yellow solid. The product is dissolved in pyridine (3 mL) and sulfur trioxide pyridine complex (0.128 g, 0.81 mmol) is added to the stirring solution. The resulting heterogeneous solution is stirred for 5 min then quenched with 7% ammonium hydroxide (aq) (15 mL) and the solution is stirred for an additional 5 min. The solvents are evaporated to dryness and the residue is re-dissolved in 7% ammonium hydroxide (aq) (25 mL) and evaporated to dryness. The resulting solid is HPLC purified to give 0.02 g (16%-2 steps) of an off-white solid.

Mixture of rotational isomers. ¹H NMR (D₂O) δ 7.7-7.5 (m, 2H), 7.4-7.2 (m, 2H), 7.0-6.8 (m, 3H), 4.6-4.2 (m, 3H), 3.0-2.4 (m, 6H), 2.39 (d, 3H). ESI-MS (m/z): 460[M−H]⁻. Anal. Calcd. for C₂₁H₂₃N₃O₇S.1.25 NH₃.2.0H₂O: C, 48.62; H, 5.97; N, 11.47. Found: C, 48.34; H, 5.48; N, 11.62.

### Example 25

### (S)-{2-[3-(3-Acetylsulfamoyl-phenyl)-propionyl]-3-phenethylcarbamoyl-1,2,3,4-tetrahydro-isoquinolin-7-yl}-sulfamic Acid

(S)-7-Nitro-3,4-dihydro-1H-isoquinoline-2,3-dicarboxylic acid 2-tert-butyl ester (0.5 g, 1.55 mmol) is dissolved in DMF/DCM (2:1). HOBt (0.261 g, 1.7 mmol) is added and the reaction cooled to 0° C. EDCI (326 mg, 1.7 mmol) is added and the reaction is stirred for 30 min at 0° C. Phenethylamine (0.28 mL, 2.3 mmol) is dissolved in DCM (1 mL) and is added to the cooled reaction mixture and the resulting solution is allowed to stir while warming to RT. The reaction is quenched with dil. HCl, and extracted with ethyl acetate. The organic layer is collected, washed with brine and dried over anhydrous Na₂SO₄. The crude reaction mixture is oncentrated and purified by flash column chromatography (1:1 EtOAc/Hex.). Yield 0.5 g (80%).

¹HNMR (300 MHz, CD₃OD) δ 1.48, 1.55 (s, 9H), 2.69 (bs, 2H), 3.20-3.34 (m, 4H), 4.57-4.62 (m, 3H), 7.11-7.25 (m, 5H), 7.37-7.40 (d, J=9 Hz, 1H), 8.09 (d, J=3.3 Hz, 2H). ¹³C NMR (75 MHz, CDCl₃) 28.57, 30.44, 31.63, 35.59, 40.53, 44.60, 53.13, 55.58, 82.13, 121.54, 122.55, 126.91, 128.78, 128.92, 129.71, 134.12, 138.62, 141.57, 146.84, 155.75, 170.55. ESI-MS (m/z): 426[M+H]⁺.

(S)-7-Nitro-3-phenethylcarbamoyl-3,4-dihydro-1H-isoquinoline-2-carboxylic Acid-tert-butyl Ester (0.380 g, 0.89 mmol) is treated with 4.0 M solution of HCl in dioxane to give the corresponding amine hydrochloride salt (0.3 g, 0.83 mmol). The salt is dissolved in DCM (2 mL) and DIPEA (0.14 mL) and added to a cooled (0° C.) reaction mixture containing the 3-(3-sulfamoyl-phenyl)-propionic acid derivative (0.158 g, 0.69 mmol), HOBt (0.116 g, 0.76 mmol) and EDCI (0.146 g, 0.76 mmol) in DCM/DMF (2:1; 3 mL). The reaction mixture is allowed to stir overnight while warming to room temperature. The reaction is quenched with dil. HCl, and extracted with ethyl acetate. The organic layer is collected, washed with sodium bicarbonate (satd. aq), brine and dried over anhydrous Na₂SO₄. The crude reaction mixture is oncentrated and purified by flash column chromatography (7:3 CHCl3/MeOH.). Yield 0.0.286 g (60%).

¹HNMR (300 MHz, CD₃OD) δ 2.66 (t, J=7.2 Hz, 2H), 2.89-3.26 (m, 8H), 4.63-4.78 (m, 2H), 5.12 (dd, J=6.0, 2.4 Hz, 1H), 7.06-7.83 (m, 9H), 8.01 (s, 1H), 8.09 (dd, J=7.7, 2.4 Hz, 2H). ESI-MS (m/z): 537[M+H]⁺.

(S)-7-Nitro-2-[3-(3-sulfamoyl-phenyl)-propionyl]-1,2,3,4-tetrahydro-isoquinoline-3-carboxylic acid phenethyl-amide(0.268 g, 0.5 mmol) is treated with acetic anhydride (0.05 mL) and DMAP (0.061 g) in acetonitrile at 0° C. After 2h the reaction mixture is diluted with 1 N HCl (25 mL) and extracted with ethyl acetate (3×25 mL). The organic fractions are pooled, washed with brine and dried over sodium sulfate. Following concentration the crude material is purified by column chromatography on silica gel (19:1 CHCl₃/MeOH). Yield 230 mg (80%).

¹HNMR (300 MHz, CD₃OD) δ 2.07 (2d, J=7.05, 1.2 Hz, 3H), 2.56 (t, J=7.2 Hz, 1H), 2.73-2.81 (m, 3H), 3.02-3.14 (m, 3H), 3.36-3.53 (m, 3H), 4.30 (d, J=15.9 Hz, 1H), 4.60 (d, J=15.9 Hz, 1H), 5.28 (t, J=3.9 Hz, 1H), 6.34 (bs, 1H), 6.96 (bs, 1H), 7.11-7.55 (m, 8H), 7.89-7.95 (m, 2H), 8.10 (d, J=8.1 Hz, 1H). ESI-MS (m/z): 579[M+H]⁺.

The acyl sulfonamide from above (0.230 g, 0.40 mmol) is dissolved in methanol and 10% Pd/C (0.100 g) is added. The resulting slurry is stirred for two hours under an atmosphere of hydrogen. The reaction mixture is filtered over Celite and the filtrate concentrated and dried under vacuum. The corresponding amine (0.179 g) is dissolved in pyridine and treated with SO₃-pyridine complex (0.158 g, 0.99 mmol). The reaction is stirred for 4-5 min and 7% NH₄OH/H₂O is added. Volatiles are removed by roary evaporation followed by co-evaporation with acetonitrile several times. The crude product is dried under vacuum and purified by RP-HPLC. Yield 0.037 g (14%-2 steps).

¹HNMR (300 MHz, D₂O) δ 1.75 (d, J=15.6 Hz, 3H), 2.37-3.20 (m, 10H), 4.09-4.34 (m, 3H), 6.74 (br.s, 1H), 6.83-6.87 (m, 4H), 7.02-7.16 (m, 3H), 7.37 (dd, J=13.5, 7.5 Hz, 2H), 7.59 (dd, J=8.4, 2.7 Hz, 2H), Anal. Calcd for C₂₉H₃₂N₄O₈S₂ NH₃.2H₂O: C, 51.09; H, 5.77; N, 10.27. Found: C, 51.14; H, 5.79; N, 10.27. ESI-MS (m/z): 627[M−H]⁻.

### Example 26

### (S)-(2-Benzoyl-3-methylcarbamoyl-1,2,3,4-tetrahydroisoquinolin-7-yl)-sulfamic Acid

(S)-2-Benzoyl-7-nitro-1,2,3,4-tetrahydro-isoquinoline-3-carboxylic acid methylamide (0.155 g, 0.46 mmol) is dissolved in methanol (5 mL) under nitrogen and 10% Pd/C (0.075 g) is added to this stirring solution. The resulting slurry is stirred under an atmosphere of hydrogen for 5h. The slurry is filtered through Celite, concentrated and vacuum dried. The dry, crude amine is dissolved in dry pyridine (1.5 mL) and to this is added sulfur trioxide pyridine complex (0.15 g). The homogeneous reaction is stirred for 5 min before adding a 7% NH₄OH/H₂O solution (20 mL). All volaitles are removed and the crude sulfamic acid is vacuum dried for 18 h. before purifying by RP-HPLC. Yield 0.057 g (29%-2 steps) Mixture of rotational isomers. ¹H NMR (D₂O) δ 7.3-7.5 (m, 4H), 7.25-6.7 (m, 4H), 4.74.35 (m, 3H), 3.2-2.9 (m, 2H), 2.51, 2.36 (s, 3H). ¹³C{¹H} NMR (D₂O) δ 174.88, 174.40, 174.15, 173.76, 139.34, 139.16, 135.19, 134.89, 134.70, 133.54, 131.21, 130.80, 129.35, 129.16, 129.02, 128.72, 128.57, 127.36, 127.29, 126.22, 119.07, 118.69, 117.49, 117.15, 59.08, 59.97, 49.19, 45.02, 31.74, 31.05, 26.16, 26.11. ESI-MS (m/z): 387.9[M−H]⁻. Anal. Calcd. For C₁₈H₁₉N₃O₅S.1 NH₃.1H₂O: C, 50.93; H, 5.70; N, 13.20. Found: C, 51.07; H, 5.54; N, 13.37.

### Example 27

### (S)-{2-[3-(3-Chloro-phenyl)-propionyl]-3-methylcarbamoyl-1,2,3,4-tetrahydro-isoquinolin-7-yl}-sulfamic Acid

3-Chlorocinnamic acid (0.202 g, 1.1 mmol) is dissolved in dichloromethane (3 mL). EDC (0.213, 1.1 mmol) is added and the reaction is stirred for 30 min. Diisopropyl ethylamine (0.258 mL, 1.5 mmol) is added to the stirring solution followed by (S)-3-methylcarbamoyl-7-nitro-1,2,3,4-tetrahydro-isoquinoline hydrochloride (0.20 g, 0.74 mmol) and the reaction is stirred for 18 h. Ethyl acetate (20 mL) is added and the solution is washed with 1N sodium hydroxide (3×10 mL), water (10 mL), 0.1N hydrochloric acid (3×10 mL) and brine (10 mL). The organic layer is dried over magnesium sulfate, filtered and evaporated to give a yellow solid. Yield 0.256 g (87%). ESI-MS m/z: 398[M−H]⁻. The product is dissolved in ethanol (3 mL) and degassed with argon for 2 min. 10% Palladium on carbon (0.08 g) is added and hydrogen is bubbled through the stirring solution for 3 h. The reaction mixture is filtered through celite and the solvent is evaporated to give a yellow solid. Yield 0.235 g (99%). ESI-MS m/z: 368[M−H]⁻. The product is dissolved in pyridine (3 mL) and sulfur trioxide pyridine complex (0.310 g, 2.1 mmol) is added to the stirring solution. The resulting heterogeneous solution is stirred for 5 min then quenched with 7% ammonium hydroxide (aq) (25 mL) and the solution is stirred for an additional 5 min. The solvents are evaporated and the residue is re-dissolved in 7% ammonium hydroxide (aq) (25 mL) and evaporated to dryness. The resulting solid is purified by RP-HPLC to give an off-white solid. Yield 0.124 g (42%) (36% over 3 steps). ¹H NMR (D₂O) δ 7.20-6.82 (m, 7H), 4.434.20 (m, 3H), 2.95-2.50 (m, 6H), 2.43 (d, 3H). ESI-MS (m/z): 451.9[M+H]⁺. Anal. Calcd. for C₂₀H₂₂N₃O₅S.3/4 NH3 1H₂O: C, 49.76; H, 5.48; N, 10.88. Found: C, 49.35; H, 5.22; N, 10.99.

### Examples 28-34

The following chemical formula along with Table 2 shows the structure of compounds made according to the description in Examples 28-34 below:

TABLE 2

| | |
|---|---|
| | Formula (IV) |
| 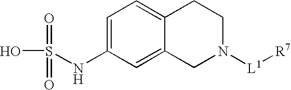 | |

| EXAMPLE | L¹ | L⁷ |
|---|---|---|
| 28 | —CO₂— | 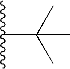 |
| 29 | —CO₂— | 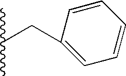 |
| 30 | —CONH— | 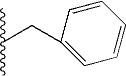 |
| 31 | —CO— | 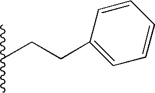 |
| 32 | —CO— | 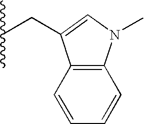 |
| 33 | —SO₂— | 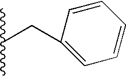 |
| 34 | —CO— | 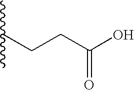 |

### Example 28

### 7-Sufoamino-3,4-dihydro-1H-isoquinoline-2-carboxylic Acid Tert-Butyl Ester

4-Nitrophenethylamine hydrochloride (2.48 g, 12 mmol) is slurried in dichloromethane (15 mL) and triethylamine (3.5 mL, 25 mmol) are added. The mixture is agitated at room temperature until homogeneous at which point it is cooled to 0° C. and trifluoroacetic acid anhydride (1.66 mL, 12 mmol) is added slowly via syringe over a period of 10 min. The reaction is allowed to warm to room temperature over a period of 2h then diluted with dichloromethane (75 mL). The organics are washed successively with H₂O (3×100 mL), 1N HCl (1×100 mL) then brine (2×100 mL) then dried over MgSO₄ and evaporated to dryness to give a yellow solid. Yield 2.81 g (90%).

¹H NMR (CDCl₃) δ 8.19 (dd, J=8.8, 1.8 Hz, 2H), 7.40 (dd, J=8.8, 1.8 Hz, 2H), 3.70 (dt, J=7.0, 6.6 Hz, 2H), 3.06 (t, J=7 Hz, 2H). 3C{I H} NMR (CDCl₃) δ 158.87 (qt, J_{C-F}=37.2 Hz), 148.37, 146.88, 131.08, 125.41, 117.10 (qt, J_{C-F}=287.8 Hz), 41.96, 36.27. MS m/z 261[M−H]⁻.

### 2,2,2-Trifluoro-1-(7-nitro-3,4-dihydro-1H-isoquinolin-2-yl)ethanone

2,2,2,-Trifluoro-N-[2-(4-nitrophenyl)ethyl]acetamide (2.4 g, 9.15 mmol) and paraformaldehyde (450 mg) are added at room temperature to a mixture of acetic acid (10 mL) and sulfuric acid (15 mL). The mixture is magnetically stirred for 22 h. The reaction mixture is poured into an ice/H₂O mixtue (200 mL). Upon warming, the aqueous mixture is extracted with ethyl acetate (3×100 mL). The organics are pooled and washed successively with saturated, aqueous sodium bicarbonate solution (3×100 mL) then brine (2×100 mL). The ethyl acetate layer is dried over magnesium sulfate, concentrated and vacuum dried for 24 h to give a yellow solid. Yield 2.17 g (86%).

Mixture of rotational isomers: ¹H NMR (CDCl₃) δ 8.0-8.2 (m, 2H), 7.38 (tr, 1H, J=7.7 Hz), 5.9 (d, 2H), 4.9 (m, 2H), 3.1 (m, 2H). ¹³C{¹H} NMR (CDCl₃) δ 155.77 (qt, J_{C-F}=36.3 Hz), 155.65 (qt, J_{C-F}=36.3 Hz), 146.66, 146.57, 141.49, 140.71, 133.02, 132.83, 129.99, 129.76, 122.34, 121.91, 121.71, 121.25, 116.22 (qt, J_{C-F}=287.7 Hz), 46.62, 46.59, 45.08, 42.61, 42.59, 40.96, 29.28, 27.90. MS m/z 273[M−H]⁻.

### 7-Nitro-1,2,3,4-tetrahydro Isoquinoline

2,2,2-Trifluoro-1-(7-nitro-3,4-dihydro-1H-isoquinolin-2-yl)ethanone (0.620 g, 2.26 mmol) is dissolved in methanol (45 mL) and added to a solution of potassium carbonate (1 g, 7.2 mmol) dissolved in H₂O (5 mL). The reaction is allowed to stir for 60 min then evaporated to dryness. The residue is redissolved in dichloromethane (100 mL) and H₂O (50 mL) and the organic layer is drawn off. The H₂O layer is washed with dichloromethane (3×100 mL) followed by 3:1 dichloromethane/iso-propanol (4×50 mL). Organic fractions pooled and dried over magnesium sulfate to give an orange solid. Yield 0.4 g (98%).

¹H NMR (CD₃OD) δ 7.7 (t, 2H), 7.1 (d, 1H), 4.7 (s, 2H), 2.8 (t, 2H), 2.7 (t, 2H). ¹³C{¹H} NMR (CD₃OD) δ 147.87, 144.62, 138.59, 131.80, 122.83, 122.40, 48.86. MS m/z 179[M−H]⁺.

### 7-Sufoamino-3,4-dihydro-1H-isoquinoline-2-carboxylic Acid Tert-Butyl Ester

7-Nitro-1,2,3,4-tetrahydro isoquinoline (0.20 g, 1.1 mmol) is dissolved in acetonitrile (3 mL) and to this is added di-t-butyl dicarbonate (0.262 g, 1.2 mmol) as a solution in methylene chloride (2 mL) (evolution of gas observed). The reaction mixture is stirred at room temperature for 2 h before adding DMAP (5 mg) and additional di-t-butyl dicarbonate (0.360 g, 1.65 mmol). After 1 h the reaction mixture is evaporated to dryness and re-dissolved in ethyl acetate (50 mL) and washed with 1N HCl (aq) (3×30 mL) followed by brine (2×30 mL). The ethyl acetate layer is collected, dried over magnesium sulfate and purified by flash column chromatography on silica gel eluting with 5:1 hexanes/ethyl acetate. Yield 0.254 g (83%).

¹H NMR (CDCl₃) δ 8.0 (m, 2H), 7.3 (d, 1H), 4.7 (s, 2H), 3.7 (t, 2H), 2.9 (t, 2H), 1.5 (s, 9H).

¹³C{¹H} NMR (CDCl₃) δ 154.83, 146.69, 142.72, 135.78, 130.02, 121.77, 121.58, 80.61, 45.94, 41.56, 29.43, 28.66.

7-Nitro-3,4-dihydro-1H-isoquinoline-2-carboxylic acid tert-butyl ester (0.23 g, 0.831 mmol) is dissolved in ethanol (8 mL) and degassed with argon for several minutes. 10% Palladium on carbon (0.05 g) is added and the heterogeneous reaction mixture is placed on the Parr apparatus and subjected to hydrogen at a pressure of 40 psi. The hydrogenation is allowed to proceed for 4 h at which point the reaction is filtered through Celite and concentrated to give a light purple oily residue. MS m/z 249[M+H]⁺.

7-Amino-3,4-dihydro-1H-isoquinoline-2-carboxylic acid tert-butyl ester (0.831 mmol) is dissolved in pyridine (1 mL) and to this is added sulfur trioxide pyridine comple (0.390 g, 2.45 mmol) and the reaction is allowed to stir for 5 min. 7% Ammonium hydroxide solution (aq) is added (15 mL) and the reaction is concentrated to give a pink residue. The crude material is purified by RP-HPLC. Yield 0.075 g (26%).

¹H NMR (D₂O) δ 7.08 (d, J=8.4 Hz, 1H), 7.00 (dd, J=8.4, 2.1 Hz, 1H), 6.93 (s, 1H), 4.48 (br, 2H), 3.53 (dd, J=5.9, 5.5 Hz, 2H), 2.71 (dd, J=5.9, 5.5 Hz, 2H), 1.43 (s, 9H). ¹³C{¹H} NMR (D₂O) δ 156.82, 138.23, 134.14, 129.58, 118.19, 117.07, 81.93, 45.60, 41.96, 27.90, 27.61. MS m/z 327[M−H]⁻. Anal. Calcd. for C₁₄H₂₃N₃O₅S.1.1H₂O: C, 46.04; H, 6.95; N, 11.51. Found: C, 45.87; H, 6.31; N, 11.09.

### Example 29

### 7-Sulfoamino-3,4-dihydro-1H-isoquinoline-2-carboxylic Acid Benzyl Ester

7-Nitro-1,2,3,4-tetrahydro isoquinoline (0.20 g, 1.1 mmol) is dissolved in 1,4-dioxane (2.5 mL) and 1N NaOH (2 mL) is added followed by benzyl chloroformate (170 uL, 1.2 mmol). The reaction is stirred for 1 h. The reaction is acidified to pH 1 with 1N HCl and diluted with ethyl acetate (60 mL). The organic layer is washed with 1N HCl (2×25 mL) followed by brine (1×25 mL) and dried over magnesium sulfate. Following concentration the crude material is purified by flash column chromatography on silica gel eluting with 3:1 hexanes/ethyl acetate to give a yellow solid upon vacuum drying. Yield 0.27 g (79%).

¹H NMR (CDCl₃) δ 8.0 (m, 2H), 7.4 (m, 6H), 5.2 (s, 2H), 4.7 (t, 2H), 3.8 (t, 2H), 2.9 (s, 2H). MS m/z 313[M+H]⁺.

7-Nitro-3,4-dihydro-1H-isoquinoline-2-carboxylic acid benzyl ester (0;27 g, 0.86) is dissolved in ethyl acetate (4 mL) and to this is added a solution of SnCl₂.2H₂O (1 g, 4.3 mmol) dissolved in ethanol. The reaction is heated to 50° C. for 2 h then to 70° C. for an additional 2 h. Upon cooling the reaction is diluted with ethyl acetate and washed with 1N NaOH (4×30 mL) followed by brine (2×30 mL) and dried over magnesium sulfate. Yield 0.211 g MS m/z 283[M+H]⁺.

7-Amino-3,4-dihydro-1H-isoquinoline-2-carboxylic acid benzyl ester (0.10 g, 0.35) is dissolved in pyridine (1 mL) and sulfur trioxide pyridine complex (0.17 g, 1.06 mmol) is added in one portion. The reaction is allowed to stir for 5 min then quenched with 7% ammonium hydroxide (aq) (10 mL). The reaction is concentrated and purified by RP-HPLC. Yield 0.068 g (52%).

¹H NMR (D₂O) δ 7.36 (s, 5H), 7.08 (d, J=8.3 Hz, 1H), 7.00 (d, J=8.3 Hz, 1H), 6.90 (s, 1H), 5.10 (s, 2H), 4.51 (br, 2H), 3.54 (t, J=5.9 Hz, 2H), 2.68 (br, 2H). ¹³C{¹H} NMR (D₂O) δ 156.20, 138.65, 136.68, 133.63, 129.34, 128.68, 128.23, 127.94, 118.14, 117.07, 67.48, 45.61, 41.80, 27.71. MS m/z 361[M−H]⁻. Anal. Calcd. for C₁₇H₂₁N₃O₅S.2/3H₂O: C, 52.16; H, 5.75; N, 10.73. Found: C, 52.40; H, 5.5; N, 10.55.

### Example 30

### 2-(Benzylcarbamoyl-1,2,3,4-tetrahydro-isoquinolin-7-yl)-sulfamic Acid

7-Nitro-1,2,3,4-tetrahydro isoquinoline (0.15 g, 0.83 mmol) is dissolved in dichloromethane (2.5 mL) and benzylisocyanate (0.104 mL, 0.83 mmol) is added The reaction is stirred for about 10 min before formation of a white ppt is observed. Additional dichloromethane (1 mL) is added and the reaction is stirred for 18 h. The precipitate is filtered off and washed with hexanes several times. The filtrate is treated with PS-polyamine resin (1.0 g, 4.5 mmol, Novabiochem) and the slurry is stirred for 18 hr. The resin is filtered off and the precipitate from above is added to the filtrate and this combined mixture is evaporated to dryness to yield a white solid. Yield 0.250g (97%).

¹H NMR (CDCl₃) δ 8.0 (m, 2H), 7.3 (m, 6H), 5.0 (s, 1H), 4.7 (s, 2H), 4.5 (d, 2H), 3.7 (t, 2H), 3.0 (s, 2H). ¹³C{¹H} NMR (CDCl₃) δ 157.44, 146.80, 142.90, 139.43, 135.27, 129.81, 128.95, 128.08, 127.72, 121.96, 121.84, 45.76, 45.38, 41.06, 29.46. MS m/z 312[M+H]⁺.

7-Nitro-3,4-dihydro-1H-isoquinoline-2-carboxylic acid benzylamide (0.216 g, 0.7 mmol) is dissolved in 1:1 ethyl acetate/ethanol (7 mL) and degassed with argon for several minutes. 10% Palladium on carbon (0.05 g) is added and hydrogen is introduced into the flask via balloon. The reaction is agitated for 2.5 h before filtering through celite and concentrating to give an off-white solid. Yield 0.192 g (98%).

¹H NMR (CD₃OD) δ 7.3 (s, 4H), 7.2 (t, 1H), 7.0 (d, 1H), 6.6 (d, 1H), 6.5 (s, 1H), 4.5 (s, 2H), 4.4 (s, 2H), 3.6 (t, 2H), 2.8 (t, 2H). ¹³C {¹H} NMR (CD₃OD) δ 159.14, 145.91, 140.64, 134.29, 129.06, 128.36, 127.17, 126.81, 124.82, 114.73, 113.01, 45.83, 44.26, 42.13, 28.02. MS m/z 282[M+H]⁺.

7-Amino-3,4-dihydro-1H-isoquinoline-2-carboxylic acid benzylamide (0.17 g, 0.61) is dissolved in pyridine (2 mL) and sulfur trioxide pyridine complex (0.3 g, 1.82 mmol) is added in one portion. The reaction is allowed to stir for 5 min then quenched with 7% ammonium hydroxide (aq) (10 mL). The reaction is concentrated and purified by RP-HPLC. Yield 0.189 g (82%).

¹H NMR (D₂O) δ 7.30 (m, 5H), 6.08 (m, 1H), 7.03 (m, 1H), 6.93 (s, 1H), 4.39 (d, J=5.9 Hz, 2H), 4.32 (s, 2H), 3.47 (br, 2H), 2.70 (br, 2H).). ¹³C{¹H} NMR (D₂O) δ 159.39, 140.00, 138.22, 134.15, 129.94, 129.39, 128.78, 127.21, 127.04, 118.25, 117.12, 45.53, 44.00, 41.63, 27.55. MS m/z 360[M−H]⁻. Anal. Calcd. for C₁₇H₂₂N₄O₄S.1/2H₂O: C, 52.70; H, 5.98; N, 14.46. Found: C, 52.64; H, 5.86; N, 14.31.

### Example 31

### [2-(3-Phenyl-propionyl)-123,4-tetrahydro-isoquinolin-7-yl]-sulfamic Acid

7-Nitro-1,2,3,4-tetrahydro isoquinoline (0.116 g, 0.65 mmol) is dissolved in dichloromethane (2.5 mL) and diisopropylethyl amine (0.14 mL, 0.78 mmol) is added followed by hydrocinnamoyl chloride (0.08 mL, 0.72 mmol). The reaction is stirred for 2 h and PS-polyamine resin (0.8 g, 3.6 mmol) is added and the reaction is continued to stir for 24 h. The resin is filtered off and the remaining filtrate is concentrated and re-dissolved in ethyl acetate (25 mL) and washed with water (2×20 mL) followed by brine (2×20 mL). The filtrate is dried over magnesium sulfate and concentrated to give a yellow residue. The crude material is purified by flash column chromatography on silica gel eluting with 1:1 hexanes/ethyl acetate. Yield 0.163 g (82%) MS m/z 311 [M+H]⁺.

1-(7-Nitro-3,4-dihydro-1H-isoquinoline-2-yl)-3-phenyl-propan-1-one (0.163 g, 0.52 mmol) is dissolved in 1:1 ethyl acetate/ethanol (3 mL) and degassed with argon for several minutes. 10% Palladium on carbon (0.05 g) is added and hydrogen is introduced into the flask via balloon. The reaction is agitated for 1 h before filtering through celite and concentrating to give an off-white solid. Yield 0.130 g (90%) MS m/z 281[M+H]⁺.

1-(7-Amino-3,4-dihydro-1H-isoquinoline-2-yl)-3-phenyl-propan-1-one (0.120 g, 0.43 mmol) is dissolved in pyridine(1.5 mL) and sulfur trioxide pyridine complex (0.3 g, 1.82 mmol) is added in one portion. The reaction is allowed to stir for 4 min then quenched with 7% ammonium hydroxide (aq) (10 mL). The reaction is concentrated and purified by RP-HPLC. Yield 0.23 g (76%).

Mixture of rotational isomers: ¹H NMR (D₂O) δ 7.1-6.8 (m, 8H), 4.4 (s, 1H), 4.2 (s, 1H), 3.4 (m, 1H), 3.3 (m, 1H), 2.7-2.4 (m, 6H). ¹³C{¹H} NMR (D₂O) δ 174.55, 174.39, 140.97, 140.23, 138.62, 138.33, 133.78, 133.52, 129.55, 129.45, 129.06, 128.90, 128.83, 128.60, 126.84, 126.76, 118.46, 118.40, 117.36, 117.25, 48.21, 44.71, 44.29, 41.07, 34.79, 34.73, 31.94, 31.65, 28.11, 27.44. MS m/z 359[M−H]⁻. Anal. Calcd. for C₁₈H₂₃N₃O₄S.1/3H₂O: C, 56.38; H, 6.22; N, 10.96. Found: C, 56.13; H, 6.13; N, 10.84.

### Example 32

### {2-[2-(1-methyl-1H-indol-3-yl)-acetyl]-1,2,3,4-tetrahydro-isoquinolin-7-yl}-sulfamic Acid

1-Methyl-3-indole-acetic acid (0.255 g, 1.35 mmol) and HOBt (0.207 g, 1.35 mmol) are dissolved in dichloromethane (5 mL) and cooled to 0° C. EDCI (0.258 g, 1.35 mmol) is added in one portion and the reaction is allowed to stir at 0° C. for 1 h until the reaction solution became homogeneous. 7-Nitro-1,2,3,4-tetrahydro isoquinoline (0.20 g, 1.1 mmol) is added in one portion to this stirring cold solution and the reaction is allowed to stir for 18 h while slowly warming to room temperature. The reaction is diluted with dichloromethane (20 mL) and washed with water (2×20 ml) followed brine (2×20 mL). The organic layer is dried over magnesium sulfate and concentrated to give a yellow-orange fluffy solid. The crude material is purified by flash column chromatography on silica gel eluting with 19:2.5 chloroform/methanol. Yield 0.135 g (29%) MS m/z 350[M+H]⁺.

2-(1-Methyl-1H-indol-3-yl)-1-(7-nitro-3,4-dihydro-1H-isoquinolin-2-yl)-ethanone (0.135 g, 0.39 mmol) is dissolved in 1:1 ethyl acetate/ethanol (3 mL) and degassed with argon for several minutes. 10% Palladium on carbon (0.05 g) is added and hydrogen is introduced into the flask via balloon. The reaction is agitated for 2 h before filtering through celite and concentrating to give an off-white solid. Yield 0.120 g (97%) MS m/z 320[M+H]⁺.

2-(1-Methyl-1H-indol-3-yl)-1-(7-amino-3,4-dihydro-1H-isoquinolin-2-yl)-ethanone (0.120 g, 0.38 mmol) is dissolved in pyridine (1.5 mL) and sulfur trioxide pyridine complex (0.18 g, 1.14 mmol) is added in one portion. The reaction is allowed to stir for 4 min then quenched with 7% ammonium hydroxide (aq) (10 mL). The reaction is concentrated and purified by RP-HPLC. Yield 0.102 g (67%).

Mixture of rotational isomers: ¹H NMR (D₂O) δ 7.3-6.5 (m, 8H), 4.2 (s, 2H), 3.5-3.3 (m, 6H), 3.0 (br. s, 1H), 2.4 (d, 1H), 1.9 (d, 1H) ¹³C{¹H} NMR (D₂O) δ 174.55, 174.40, 138.58, 138.39, 137.01, 133.59, 133.45, 129.41, 129.19, 128.85, 128.58, 128.39, 127.37, 127.28, 122.00, 119.37, 118.80, 118.10, 117.13, 116.60, 112.29, 110.08, 106.77, 106.62, 48.17, 44.70, 44.14, 41.26, 32.19, 31.13, 30.73, 27.86, 27.19. MS m/z 398[M−H]⁻. Anal. Calcd. for C₂₀H₂₄N₄O₄S.1/2H₂O: C, 56.45; H, 5.92; N, 13.17. Found: C, 56.86; H, 5.73; N, 13.16.

### Example 33

### 2-(Phenylmethanesulfonyl-1,2,3,4-tetrahydro-isoquinolin-7-yl)-sulfamic Acid

7-Nitro-1,2,3,4-tetrahydro isoquinoline (0.135g, 0.76 mmol) is dissolved in dichloromethane (2.5 mL) and diisopropylethyl amine (0.26 mL, 0.1.52 mmol) is added followed by α-toluenesulfonyl chloride (0.144 mL, 0.76 mmol). The reaction is allowed to stir for 2.5 h then diluted with dichloromethane (20 mL)and washed in succession with 1N HCl (2×25 mL) and brine (2×25 mL). The organics are dried over magnesium sulfate and concentrated to give a yellow residue, which is purified by flash column chromatography eluting with 7:3 hexanes/ethyl acetate to provide a white solid. Yield 0.15 g (60%).

¹H NMR (CDCl₃) δ8.0 (d, 1H), 7.8 (s, 1H), 7.4 (m, 5H), 7.3 (m, 1H), 4.3 (m, 4H), 3.5 (t, 2H), 2.8 (t, 2H). ¹³C{¹H} NMR (CDCl₃) 8146.82, 141.62, 134.15, 131.01, 130.52, 129.40, 129.28, 129.06, 122.14, 121.75, 58.41, 47.52, 43.47, 29.80. MS m/z 331[M−H]⁻.

7-Nitro-2-phenylmethanesulfonyl-1,2,3,4-tetrahydro isoquinoline (0.150 g, 0.45 mmol) is dissolved in 1:1 ethyl acetate/ethanol (10 mL) (slow process) and degassed with argon for several minutes. 10% Palladium on carbon (0.08 g) is added and hydrogen is introduced into the flask via balloon. The reaction is agitated for 4 h before filtering through celite and concentrating to give an off-white solid. Yield 0.051 g (38%) MS m/z 303[M+H]⁺.

7-Amino-2-phenylmethanesulfonyl-1,2,3,4-tetrahydro isoquinoline (0.051 g, 0.17 mmol) is dissolved in pyridine (1.5 mL) and sulfur trioxide pyridine complex (0.078 g, 0.5 mmol) is added in one portion. The reaction is allowed to stir for 4 min then quenched with 7% ammonium hydroxide (aq) (10 mL). The reaction is concentrated and purified by RP-HPLC. Yield 0.50 g (79%).

¹H NMR (D₂O) δ 7.4 (m, 5H), 7.1 (d, 1H), 7.0 (d, 1H), 6.8 (s, 1H), 4.5 (s, 2H), 4.3 (s, 2H), 3.4 (t, 2H), 2.7 (t, 2H). ¹³C {¹H}NMR (D₂O) δ 138.44, 133.06, 131.14, 130.16, 129.31, 129.20, 128.56, 128.45, 118.64, 116.97, 56.42, 47.30, 44.11, 27.84. MS m/z 381[M−H]⁻. Anal. Calcd. for C₁₆H₂₁N₃O₅S₂.1/4H₂O: C, 47.57; H, 5.36; N, 10.40. Found: C, 47.79; H, 5.10; N, 10.52.

### Example 34

### 4-Oxo-4-(7-sulfoamino-3,4-dihydro-1H-isoquinolin-2-yl)-butyric Acid

7-Nitro-1,2,3,4-tetrahydro isoquinoline (0.27g, 0.1.5 mmol) is dissolved in dichloromethane (3 mL) and diisopropylethyl amine (0.26 mL, 0.15 mmol) is added followed by succinic anhydride (0.150 mL, 1.5 mmol). The reaction is allowed to stir for 3 h. The reaction is diluted with dichloromethane (25 mL) and washed with 1N HCl (4×40 mL), brine (1×50 mL) and dried over magnesium sulfate. The crude material is purified by flash column chromatography eluting with 9:1 chloroform/methanol to provide a yellow solid. Yield 0.255 g (61%) Mixture of rotational isomers. ¹H NMR (CDCl₃) δ 8.0 (m, 2H), 7.3 (m, 1H), 4.8 (d, 2H), 3.9 (t, 1H), 3.8 (t, 1H), 3.1 (t, 1H), 2.9 (t, 1H), 2.7 (s, 4H). ³C{¹H} NMR (CDCl₃) δ 177.47, 170.97, 146.96, 146.76, 143.00, 141.90, 135.19, 134.12, 130.26, 129.71, 122.22, 122.14, 121.92, 121.70, 47.16, 44.41, 42.71, 39.60, 29.71, 29.50, 29.41, 28.93, 28.66, 28.36. MS m/z 277[M−H]⁻.

4-(7-Nitro-3,4-dihydro-1H-isoquinolin-2-yl)-4-oxo-butyric acid (0.26 g, 0.92 mmol) is dissolved in 1,4-dioxane (6 mL) and degassed with argon for several minutes. 10% Palladium on carbon (0.06 g) is added and hydrogen is introduced into the flask via balloon. The reaction is agitated for 2.5 h before filtering through celite and concentrating to give an yellow residue. MS m/z 247[M−H]⁻.

4-(7-Amino-3,4-dihydro-1H-isoquinolin-2-yl)-4-oxo-butyric acid (0.92 mmol) is dissolved in pyridine (1.5 mL) and sulfur trioxide pyridine complex (0.44 g, 2.76 mmol) is added in one portion. The reaction is allowed to stir for 4 min then quenched with 7% ammonium hydroxide (aq) (20 mL). The reaction is concentrated and purified by RP-HPLC. Yield 0.10 g (30%).

Mixture of rotational isomers: ¹H NMR (D₂O) δ 7.2 (d, 1H), 7.0 (t, 2H), 4.7 (s, 1H), 4.6 (s, 1H), 3.7 (m, 2H), 2.9-2.7 (m, 4H), 2.5 (t, 2H). ¹³C{¹H} NMR (D₂O) δ 180.63, 180.55, 173.99, 138.39, 138.33, 133.90, 133.70, 129.94, 129.66, 129.47, 129.34, 118.43, 118.24, 117.26, 117.10, 47.34, 44.56, 43.83, 40.79, 31.90, 31.81, 29.61, 29.38, 28.03, 27.31. MS m/z 327[M−H]⁻. Anal. Calcd. for C₁₃H₂₂N₄O₆S: C, 43.08; H, 6.12; N, 15.46. Found: C, 43.15; H, 6.16; N, 15.21.

### Examples 35-37

The following chemical formula along with Table 3 shows the structure of compounds made according to the description in Examples 37-38 below:

TABLE 3

| | |
|---|---|
| | Formula (V) |
| 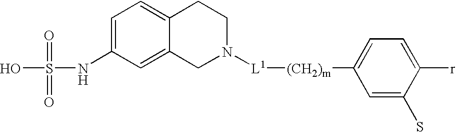 | |

| Example | L¹ | m | s | r |
|---|---|---|---|---|
| 35 | —CO— | 2 | 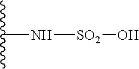 | Nil |
| 36 | —CO— | 2 | 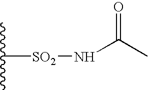 | Nil |
| 37 | —CO— | 2 | 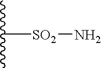 | Nil |

### Example 35

### {2-[3-(3-Sulfoamino-phenyl)-propionyl]-1,2,3,4-tetrahydro-isoquinolin-7-yl}-sulfamic Acid

3-(3-Nitro-phenyl)-propionic acid (0.360 g, 1.85 mmol) and HOBt (0.168 g, 1.85 mmol) are dissolved in 3:1 DMF/dichloromethane (6 mL) and cooled to 0° C. EDCI (0.355 g, 1.85 mmol) is added in one portion and the reaction is allowed to stir at 0° C. for 1 h. 7-Nitro-1,2,3,4-tetrahydro isoquinoline (0.30 g, 1.68 mmol) is added in one portion as a DMF solution (5 mL) to this stirring cold solution and the reaction is allowed to stir for 18 h while slowly warming to room temperature. The reaction is evaporated to near dryness and re-dissolved in ethyl acetate (25 mL). The ethyl acetate solution is washed with 0.1 N HCl (3×20 ml) followed by brine (2×20 mL). The organic layer is dried over magnesium sulfate and concentrated to give a yellow-orange solid. The crude material is purified by flash colurn chromatography on silica gel eluting with 40:1 chloroform/ethanol. Yield 0.462 g (77%) MS m/z 356[M+H]⁺.

1-(7-Nitro-3,4-dihydro-1H-isoquinolin-2-yl)-3-(3-nitro-phenyl)-propan-1-one (0.186 g, 0.524 mmol) was dissolved in diglyme (5 mL) and degassed with argon for several minutes. 10% Palladium on carbon (0.05 g) is added and hydrogen is introduced into the flask via balloon. The reaction is agitated for 2 h before filtering through celite and concentrating to give an oily brown residue. MS m/z 396[M+H]⁺.

1-(7-Amino-3,4-dihydro-1H-isoquinolin-2-yl)-3-(3-nitro-phenyl)-propan-1-one (0.524 mmol) is dissolved in pyridine (2 mL) and sulfur trioxide pyridine complex (0.5 g, 3.14 mmol) is added in one portion. The reaction is allowed to stir for 4 min then quenched with 7% ammonium hydroxide (aq) (20 mL). The reaction is concentrated and purified by RP-HPLC. Yield 0.080g (31%).

Mixture of rotational isomers: ¹H NMR (D₂O) δ 7.1-6.6 (m, 7H), 4.4 (s, 1H), 4.3 (s, 1H), 3.5 (t, 1H), 3.4 (t, 1H), 2.8-2.6 (m, 4H), 2.5 (t, 1H), 2.4 (t, 1H). ¹³C {¹H} NMR (D₂O) δ 174.47, 174.23, 142.00, 141.16, 140.37, 140.07, 138.32, 138.00, 133.38, 129.67, 129.53, 129.38, 123.15, 122.86, 119.10, 118.95, 118.30, 117.20, 48.11, 44.58, 44.22, 40.94, 34.48, 31.95, 31.60, 27.95, 27.28. MS m/z 454[M−H]⁻. Anal. Calcd. for C₁₈H₂₇N₅O₇S₂.3/4 CH₃CN: C, 45.01; H, 5.67; N, 15.48. Found: C, 44.76; H, 5.44; N, 15.68.

### Example 36

### {2-[3-(3-Acetylsulfamoyl-phenyl)-propionyl]-1,2,3,4-tetrahydro-isoquinolin-7-yl}-sulfamic Acid

3-(3-Acetylsulfamoyl-phenyl)-propionic acid (0.10 g, 0.37 mmol) and HOBt (0.068 g, 0.44 mmol) are dissolved in 1:1 DMF/DCM (2 mL) and cooled to 0° C. under nitrogen. To this cooled solution is added EDCI (0.071 g, 0.37 mmol) and the solution is stirred for 45 min. 7-Nitro-1,2,3,4-tetrahydro-isoquinoline (0.15 g, 0.84 mmol) is added as a solution in 1:1 DMF/DCM (2 mL) and the reaction is stirred for 2 h at 0° C. then allowed to slowly warm to room temperature over 18 h. The dichloromethane is evaporated and the remaining solution is diluted with ethyl acetate (50 mL) and washed with 1N HCl (3×25 mL), brine (2×25 mL) and dried over sodium sulfate. The crude material is purified by flash column chromatography (12:1 CHCl₃/MeOH). Yield 0.135 g (85%) ESI-MS (m/z): 429.9[M−H]⁻.

The resulting nitro derivative from above (0.115 g, 0.27 mmol) is desolved in 1:1 ethyl acetate/MeOH (6 mL) under nitrogen and 10% Pd/C (0.075 g) is added to this stirring solution. The resulting slurry is stirred under an atmosphere of hydrogen for 3h. The slurry is filtered through Celite, concentrated and vacuum dried. The dry, crude amine is dissolved in dry pyridine (1.5 mL) and to this is added sulfur trioxide pyridine complex (0.127 g, 0.8 mmol). The homogeneous reaction is stirred for 5 min before adding a 7% NH₄OH/H₂O solution (20 mL). All volatiles are removed and the crude sulfamic acid is vacuum dried for 18 h. before purifying by RP-HPLC. Yield 0.067 g (47%-2 steps).

Mixture of rotational isomers. ¹H NMR (D₂O) δ 7.6-6.6 (m, 7H), 4.41 (s, 1H), 4.26 (s, 1H), 3.47 (t, J=5.5 Hz, 1H), 3.30 (t, J=5.5 Hz, 1H), 2.86 (dd, 2H), 2.70 (dd, 2H), 2.49 (t, J=5.5 Hz, 1H), 2.35 (t, J=5.5 Hz, 1H), 1.78 (s, 1.5H), 1.74 (s, 1.5H). ¹³C{1H} NMR (D₂O) δ 179.29, 178.76, 174.10, 173.77, 141.78, 141.14, 141.04, 140.39, 138.34, 138.01, 133.58, 133.29, 133.15, 133.00, 129.53, 129.45, 129.26, 126.47, 124.85, 118.52, 118.29, 117.23, 48.12, 44.56, 44.18, 44.98, 34.13, 34.02, 31.62, 31.42, 27.89, 27.21, 24.72, 24.63. ESI-MS (m/z): 479.9[M−H]⁻. Anal. Calcd. For C₂₀H₂₃N₃O₇S₂.1.5 NH₃.1H₂O: C, 45.75; H, 5.66; N, 12.00. Found: C, 45.70; H, 5.53; N, 12.15.

### Example 37

### {2-[3-(3-Sulfamoyl-phenyl)-propionyl]-1,2,3,4-tetrahydro-isoquinolin-7-yl}-sulfamic Acid

3-(3-Sulfamoyl-phenyl)-propionic acid (0.27 g, 1.18 mmol) and HOBt (0.2 g, 1.3 mmol) are dissolved in 1:1 DMF/DCM (4 mL) and cooled to 0° C. under nitrogen. To this cooled solution is added EDCI (0.25 g, 1.3 mmol) and the solution is stirred for 45 min. 7-Nitro-1,2,3,4-tetrahydro-isoquinoline (0.315 g, 1.77 mmol) is added as a solution in 1:1 DMF/DCM (4 mL) and the reaction is stirred for 2 h at 0° C. then allowed to slowly warm to room temperature over 18 h. The dichloromethane is evaporated and the remaining solution is diluted with ethyl acetate (50 mL) and washed with 1N HCl (3×25 mL), brine (2×25 mL) and dried over sodium sulfate. The crude material is purified by flash column chromatography (9:1 CHCl₃/MeOH). Yield 0.424 g (92%) ESI-MS (m/z): 390[M+H]⁺.

The resulting nitro derivative from above (0.181 g, 0.46 mmol) is desolved in THF (25 mL) under nitrogen and 10% Pd/C (0.09 g) is added to this stirring solution. The resulting slurry is stirred under an atmosphere of hydrogen for 4h. The slurry is filtered through Celite, concentrated and vacuum dried. The dry, crude amine is dissolved in dry pyridine (3 mL) and to this is added sulfur trioxide pyridine complex (0.230 g, 1.34 mmol). The homogeneous reaction is stirred for 5 min before adding a 7% NH₄OH/H₂O solution (20 mL). All volatiles are removed and the crude sulfamic acid is vacuum dried for 18 h. before purifying by RP-HPLC. Yield 0.070 g (34%-2 steps).

Mixture of rotational isomers. ¹H NMR (D₂O) δ 7.6-6.6 (m, 7H), 4.53 (s, 0.75H), 4.25 (s, 1.25H), 3.53 (t, 1.5H), 3.42 (t, 0.5H), 2.94 (dd, 2H), 2.77 (dd, 2H), 2.51 (t, 1.5H), 2.42 (t, 0.5H). ESI-MS (m/z): 438[M−H]⁻. Anal. Calcd. For C₁₈H₂₁N₃O₆S₂.1 NH₃: C, 47.35; H, 5.30; N, 12.27. Found: C, 47.11; H, 5.15; N, 12.54.

### Examples 38-44

The following chemical formula along with Table 4 shows the structure of compounds made according to the description in Examples 38-44 below:

TABLE 4

| | |
|---|---|
| | Formula (VI) |
| 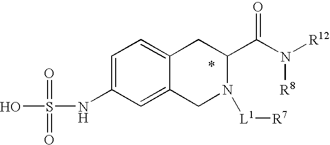 | |

| EXAMPLE | * | L¹ | R⁷ | R⁸ | R¹² |
|---|---|---|---|---|---|
| 38 | S | —CO— | 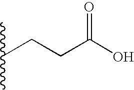 | H | —CH3 |
| 39 | S | —CO₂— | 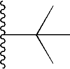 | H | 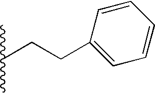 |
| 40 | S | —CO₂— | 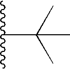 | H | 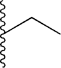 |
| 41 | S | —CO— | 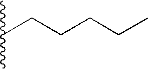 | H | 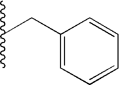 |
| 42 | S | —CO₂— | 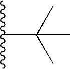 | H | 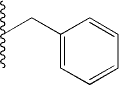 |
| 43 | R | —CO₂ | 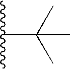 | H | —CH₃ |
| 44 | S | —CO₂ | 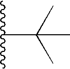 | H | —CH₃ |

### Example 38

### (S)-4-(3-Methylcarbamoyl-7-sulfoamino-3,4-dihydro-1H-isoquinolin-2-yl)-4-oxo-butyric Acid

(S)-3-Methylcarbamoyl-7-nitro-1,2,3,4-tetrahydro-isoquinoline hydrochloride (0.20 g, 0.74 mmol) is slurried in dichloroethane (3 mL). Succinic anhydride (0.085 g, 0.8 mmol) is added and the reaction is stirred for 3 h. Dichloroethane (20 mL) is added and the solution is washed with 1N sodium hydroxide (3×10 mL) and brine (10 mL). The organic layer is dried over magnesium sulfate, filtered and evaporated to give a yellow solid. Yield 0.220 g (89%). ESI-MS (m/z): 334[M−H]⁻. The product is dissolved in ethanol (3 mL) and degassed with argon for 2 min. 10% Palladium on carbon (0.08 g) is added and hydrogen is bubbled through the stirring solution for 3 h. The reaction mixture is filtered through celite and the solvent is evaporated to give a yellow solid. Yield 0.166 g (83%). ESI-MS (m/z): 304[M−H]⁻. The product is dissolved in pyridine (3 mL) and sulfur trioxide pyridine complex (0.312 g, 1.2 mmol) is added to the stirring solution. The resulting heterogeneous solution is stirred for 5 min then quenched with 7% ammonium hydroxide (aq) (25 mL) and stirred for an additional 5 min. The solvents are evaporated to dryness and the residue is re-dissolved in 7% ammonium hydroxide (aq) (25 mL) and evaporated to dryness. The resulting solid is HPLC purified to give 0.114 g (52%) (38% over 3 steps) of an off-white solid.

Mixture of rotational isomers. ¹H NMR (D₂O) δ 7.19-6.79 (m, 3H), 4.65-4.33 (m, 3H), 3.15 (m, 2H), 2.83-2.57 (m, 4H), 2.39 (m, 3H). ¹³C {¹H} NMR (D₂O) δ 181.10, 176.15, 175.89, 175.24, 174.48, 173.84, 139.15, 134.17, 133.98, 128.73, 128.64, 128.48, 127.78, 118.87, 118.59, 117.44, 57.19, 55.77, 46.54, 44.70, 32.03, 31.90, 31.70, 30.88, 30.05, 29.10, 26.22, 26.08. ESI-MS (m/z): 384[M−H]⁻. Anal. Calcd. for C₁₅H₁₉N₃O₇S.7/8 NH₃ 2 NH₃SO₃: C, 38.85; H, 5.60; N, 15.86. Found: C, 39.14; H, 5.74; N, 15.58.

### Example 39

### (S)-3-Phenethylcarbamoyl-7-sulfoamino-1,2,3,4-tetrahydro-naphthalene-2-carboxylic Acid Tert-Butyl Ester

(S)-7-Nitro-3,4-dihydro-1H-isoquinoline-2,3-dicarboxylic acid 2-tert-butyl ester (1.3 g, 0.4 mmol) is dissolved in dichloromethane (20 mL) and cooled to 0° C. EDC (0.84 g, 0.4 mmol) is added and the reaction is stirred for 30 min at 0° C. Phenethyl amine (0.55 mL, 0.4 mmol) is added and reaction is allowed to gradually warm to room temperature over a period 18 h. The solvent is evaporated and ethyl acetate (50 mL) is added. The organic layer is washed with 0.1 N hydrochloric acid (3×25 mL) and brine (25 mL). The organic layer is collected and dried over magnesium sulfate, filtered and evaporated to give and orange solid. The solid is re-dissolved in ethyl acetate (50 mL) and the organic layer is washed with potassium carbonate (aq) (3×100 mL), dried over magnesium sulfate, filtered and evaporated to give an orange solid. Yield 1.60 g (94%). ESI-MS (m/z): 394[M−H]⁻.

### (S)-3-Phenethylcarbamoyl-7-sulfoamino-1,2,3,4-tetrahydro-naphthalene-2-carboxylic Acid Tert-Butyl Ester

(S)-7-Nitro-3-phenethylcarbamoyl-1,2,3,4-tetrahydro-naphthalene-2-carboxylic acid tert-butyl ester (0.60 g, 1.4 mmol) is dissolved in methanol (10 mL) and degassed with argon for 2 min. 10% Palladium on carbon (0.10 g) is added and hydrogen is bubbled through the stirring solution for 3 h. The reaction mixture is filtered through celite and the solvent is evaporated to give a yellow solid. Yield 0.38 g (68%). ESI-MS (m/z): 394[M−H]⁻. The product (0.15 g, 0.4 mmol) is dissolved in pyridine (3 mL) and sulfur trioxide pyridine complex (0.07 g, 0.4 mmol) is added to the stirring solution. The resulting heterogeneous solution is stirred for 2 h then quenched with 7% ammonium hydroxide (aq) (25 mL) and stirred for an additional 5 min. The solvents are evaporated to dryness and the residue is re-dissolved in 7% ammonium hydroxide (aq) (25 mL) and evaporated to dryness. The resulting solid is HPLC purified to give 0.02 g (10%) of an off-white solid.

Mixture of rotational isomers. ¹H NMR (D₂O) δ 7.22 (m, 3H), 6.95 (m, 5H), 4.42-4.20 (m, 3H), 3.33-3.09 (m, 2H), 2.90 (m, 2H), 2.49 (m, 2H), 1.27 (s, 9H). ESI-MS (m/z): 474[M−H]⁻. Anal. Calcd. for C₂₃H₃₂N₄O₆S.1H₂O: C, 54.10; H, 6.71; N, 10.97. Found: C, 53.72; H, 6.41; N, 10.87.

### Example 40

### (S)-3-Ethylcarbamoyl-7-sulfoamino-1,2,3,4-tetrahydro-naphthalene-2-carboxylic Acid Tert-Butyl Ester

(S)-7-Nitro-3,4-dihydro-1H-isoquinoline-2,3-dicarboxylic acid 2-tert-butyl ester (7.6 g, 26.1 mmol) is added to a slurry of Kaiser oxime resin (9.77 g, 10.45 mmol, Novabiochem) in dichloromethane (DCM) (60 mL). The heterogeneous reaction mixture is agitated until all the acid had dissolved (ca. 20 min). Diisopropyl carbodimide (3.3 g, 26.1 mmol) is added slowly to the vessel and the reaction is allowed to agitate for 18 h while periodically venting (first 2h). The resin is filtered and washed with copius amounts of DCM, DMF and methanol in an alternating fashion. Resin vacuum dried for 20 h. Yield 13.06 g (S)-7-Nitro-3,4-dihydro-1H-isoquinoline-2,3-dicarboxylic acid 2-tert-butyl ester-3-oxime resin ester (3.0 g, 3.0 mmol) is washed with dimethyl acetamide (3×10 mL) and dimethyl acetamide (10 mL) is added. Tin (II) chloride dihydrate (6.7 g, 30.0 mmol) is added and reaction is shaken for 18 h. The resin is filtered and washed with dimethyl acetamide (3×10 mL), isopropanol (10 mL), dichloromethane (2×10 mL), isopropanol (10 mL), dichloromethane (10 mL) and isopropanol (3×10 mL). The resin is taken on to the next step without characterization. The resin is washed with pyridine (2×10 mL). Sulfur trioxide pyridine complex (1.6 g, 10.1 mmol) is dissolved in dimethyl acetamide (12 mL) and pyridine (50 mL) is added. The solution is added to the resin and the reaction is shaken for 30 min. The resin is washed with dimethyl acetamide (2×10 mL). Sulfur trioxide pyridine complex (1.6g, 10.1 mmol) is dissolved in dimethyl acetamide (12 mL) and pyridine (50 mL) is added. The solution is added to the resin and the reaction is shaken for 1 h. The resin is washed with dimethyl acetamide (3×10 mL), isopropanol (10 mL), dichloromethane (10 mL) and isopropanol (10 mL). The resin (0.20 g, 0.2 mmol) is washed with tetrahydrofuran (3×10 mL). The resin is swelled in THF (5 mL) and a solution of ethylamine in THF (1.0 mL, 2 mmol, 2M) is added and the resin is agitated for 1 h. The filtrate is collected, ammonium hydroxide (aq) (1 mL) is added and the solvents are evaporated to give a yellow solid.

Mixture of rotational isomers. Yield 0.062 g (75%). ¹H NMR (CDCl₃) δ 7.12-6.90 (m, 3H), 4.52-4.26 (m, 3H), 2.95 (m, 2H), 1.33 (d, 9H), 1.18, (m, 2H), 0.80 (m, 3H). ¹³C {¹H} NMR (CDCl₃) δ 175.12, 175.02, 157.18, 156.86, 139.13, 135.61, 135.29, 128.82, 128.42, 118.90, 117.59, 82.84, 57.46, 56.52, 45.60, 44.62, 35.26, 34.54, 31.62, 27.90, 14.00, 12.15. ESI-MS (m/z): 398[M−H]⁻. Anal. Calcd. for C₁₇H₂₅N₃O₆S.1H₂O.1/2 NH₃.1/4 CH₃CN: C, 48.18; H, 6.76; N, 12.04. Found: C, 48.28; H, 6.72; N, 12.32.

### Example 41

### (S)-(3-Benzylcarbamoyl-2-hexanoyl-1,2,3,4-tetrahydro-isoquinolin-7-yl)-sulfamic Acid

(S)-7-Nitro-1,2,3,4-tetrahydro-isoquinoline-3-carboxylic acid benzylamide trifluoroacetate (0.20 g, 0.47 mmol) is slurried in dichloromethane (3 mL) and diisopropylethylamine (0.168 mL, 1.0 mmol) is added to the stirring solution. Hexanoyl chloride (0.098 mL, 0.7 mmol) is added and the reaction is stirred for 2 h. Ethyl acetate (20 mL) is added and the solution is washed with 1N sodium hydroxide (3×10 mL) and brine (10 mL). The organic layer is dried over magnesium sulfate, filtered and evaporated to give a yellow solid. Yield 0.119 g (62%). ESI-MS (m/z): 408[M−H]⁻. The product is dissolved in ethanol (3 mL) and degassed with argon for 2 min. 10% Palladium on carbon (0.08 g) is added and hydrogen is bubbled through the stirring solution for 3 h. The reaction mixture is filtered through celite and the solvent is evaporated to give a yellow solid. Yield 0.101 g (92%). ESI-MS (m/z): 378[M−H]⁻. The product is dissolved in pyridine (3 mL) and sulfur trioxide pyridine complex (0.126 g, 0.8 mmol) is added to the stirring solution. The resulting heterogeneous solution is stirred for 5 min then quenched with 7% ammonium hydroxide (aq) (25 mL) and the solution is stirred for an additional 5 min. The solvents are evaporated to dryness and the residue is re-dissolved in 7% ammonium hydroxide (aq) (25 mL) and evaporated to dryness. The resulting solid is HPLC purified to give 0.055 g (46%) (19% over 3 steps) of an off-white solid.

Mixture of rotational isomers. ¹H NMR (D₂O) δ 7.23 (m, 3H), 7.13 (m, 3H), 6.75-6.59 (m, 2H), 4.82 (m, 1H), 4.67-3.97 (m, 4H), 3.25 (m, 1H), 2.60 (m, 2H), 1.62 (m, 2H), 1.30 (m, 2H), 0.88 (m, 3H). ¹³C {¹H} NMR (D₂O) δ 177.85, 177.64, 174.15, 173.66, 139.67, 139.52, 137.99, 137.79, 134.33, 133.99, 128.97, 128.82, 128.34, 127.48, 127.41, 126.64, 126.58, 118.90, 118.58, 117.50, 117.36, 57.75, 56.06, 46.89,45.13, 43.09, 42.84, 34.06,33.84,32.07, 31.20,31.11, 31.02, 24.71, 24.31, 22.17, 22.03, 13.60. ESI-MS (m/z): 457[M−H]⁻. Anal. Calcd. for C₂₃H₃₂N₄O₅S.1H₂O: C, 55.85; H, 6.93; N, 11.33. Found: C, 55.9; H, 6.45; N, 11.24.

### Example 42

### (S)-3-Benzylcarbamoyl-7-sulfoamino-3,4-dihydro-1H-isoquinoline-2-carboxylic Acid Tert-Butyl Ester

(S)-3-Benzylcarbamoyl-7-nitro-1,2,3,4-tetrahydro-naphthalene-2-carboxylic acid tert-butyl ester (0.80 g, 1.9 mmol) is dissolved in ethanol (20 mL) and degassed with argon for 2 min., 10% Palladium on carbon (0.5 g)is added and hydrogen is bubbled through the stirring solution for 3 h. The reaction mixture is filtered through celite and the solvent is evaporated to give a yellow solid. Yield 0.69 g (95%). ESI-MS (m/z): 380[M−H]⁻. The product (0.20 g, 0.52 mmol) is dissolved in pyridine (3 mL) and sulfur trioxide pyridine complex (0.250 g, 1.6 mmol) is added to the stirring solution. The resulting heterogeneous solution is stirred for 5 min then quenched with 7% ammonium hydroxide (aq) (25 mL) and the solution is stirred for an additional 5 min. The solvents are evaporated to dryness and the residue is re-dissolved in 7% ammonium hydroxide (aq) (25 mL) and evaporated to dryness. The resulting solid is HPLC purified to give 0.110 g (44%) of an off-white solid.

Mixture of rotational isomers. ¹H NMR (D₂O) δ 7.25-6.68 (m, 8H), 4.62-3.97 (m, 5H), 2.98 (m, 2H), 1.40 (d, 9H). ¹³C {¹H} NMR (D₂O) δ 176.34, 175.34, 157.29, 156.84, 139.40, 137.99, 135.24, 135.03, 128.98, 128.61, 128.30, 128.05, 127.55, 127.36, 127.03, 126.46, 118.72, 117.44, 82.87, 57.38, 56.50, 45.70, 44.77, 42.86, 31.58, 28.02, 27.88, 27.30. ESI-MS (m/z): 460[M−H]⁻. Anal. Calcd. for C₂₂H₃₀N₄O₆S.1/2H₂O: C, 54.19; H, 6.41; N, 11.49. Found: C, 54.26; H, 6.14; N, 11.47.

### Example 43

### (R)-7-Nitro-1,2,3,4-tetrahydro-isoquinoline-3-carboxylic Acid

H-(D)-Tic-OH (5.0 g, 28.2 mmol) is slurried in conc. H₂SO₄ (50 mL) and magnetically stirred until the reaction turned homogeneous (30 min). Reaction solution is cooled in an ice bath to <5° C. and solid potassium nitrate in small portions is added to keep the reaction temperature between 5-10° C. The reaction is allowed to stir while slowly warming to room temperature over a period of 18 h. The reaction solution is poured into ice water (750 mL) and the reaction mixture is placed into a large ice bath. The reaction is basified by the addition of conc. NH₄OH while maintaining the temperature <10° C. The resulting slurry is filtered and the yellow filter cake is washed with water then vacuum dried for 18 h. The yellow solid is dissolved in methanol (50 mL) and conc. HCl (3 mL) is added. The reaction is briefly heated to reflux (solid did not completely dissolve) then left to cool to room temperature. Cooling is continued in the freezer then is filtered and washed with cold methanol to provide a white solid. Yield 3.0 g (41%).

### (R)-7-Nitro-3,4-dihydro-1H-isoquinoline-2,3-dicarboxylic acid-2-tert-butyl Ester

(R)-7-Nitro-1,2,3,4-tetrahydro-isoquinoline-3-carboxylic acid (3.0 g, 11.5 mmol) is slurried in methanol (30 mL), water (3 mL) and triethyl amine (15 mL) and the reaction is stirred for 1 h. Di-t-butyl dicarbonate (2.9 mL, 12.7 mmol) is added to the slurry and the reaction is allowed to stir for 18 h. The reaction solution is extracted with dichloromethane (5×50 mL) and the combined dichloromethane fractions are pooled and concentrated to an oil. The oil is taken-up in 0.5 N NaOH (50 mL) and washed with diethyl ether (2×50 mL). The aqueous layer is acidified to pH˜1 with conc. HCl and extracted with diethyl ether (3×50 mL). The ether layers are dried over sodium sulfate and concentrated to give a tan foam which is further dried under vacuum for 48 h. This material is pooled with fractions from several similar runs and purified by flash column chromatography on silica gel eluting with 10% methanol in dichloromethane. Yield 11.24 g.

Mixture of rotational isomers. ¹H NMR (CDCl₃) δ 8.2 (d, 1H), 8.1 (t, 1H), 7.5 (m, 1H), 4.95 (m, 0.5H), 4.8-4.4 (m, 2.5H), 3.25 (m, 2H), 1.44 (d, 9H). ¹³C{¹H} NMR (CDCl₃) δ 173.42, 173.15, 155.12, 154.80, 146.90, 141.78, 141.26, 136.40, 135.67, 130.50, 130.07, 122.31, 80.52, 80.38, 53.90, 52.34, 44.61, 44.08, 31.77, 31.60, 28.75, 28.62. ESI-MS (m/z): 323[M+H]⁺. mp=75-80° C. [α]_{D}=−42.6 (c=1.065, CH₃OH). Anal. Calcd. for C₁₅H₁₈N₂O₆[0.1 CH₂Cl₂]: C, 54.82; H, 5.55; N, 8.47. Found: C, 54.81; H, 5.34; N, 8.55.

### (R)-3-Methylcarbamoyl-7-sulfoamino-3,4-dihydro-1H-isoquinoline-2-carboxylic Acid Tert-Butyl Ester

(R)-7-Nitro-3,4-dihydro-1H-isoquinoline-2,3-dicarboxylic acid-2-tert-butyl ester (0.24g, 0.716 mmol) is dissolved in 1:1 ethyl acetate/ethanol and the reaction vessel is purged with N₂. 10% Palladium on carbon (0.067 g) is added and the reaction is placed on the Parr shaker at 40 psi overnight. The reaction soluition is filtered through celite and concentrated to give a purplish residue. The crude material is purified by flash column chromatography on silica gel eluting with 24:1 chloroform/methanol. Yield 0.130 g (60%). ESI-MS (m/z): 306[M+H]⁺. The reduction product (0.1 g, 0.33 mmol) is dissolved in pyridine (1.5 mL) and sulfur trioxide pyridine complex (0.156 g, 0.984 mmol) is added. The reaction is stirred for 15 min before quenching with a 7% aqueous solution of ammonium hydroxide. The crude material is purified by RP-HPLC to give a pinkish solid. Yield 0.040 g (30%).

Mixture of rotational isomers. ¹H NMR (CDCl₃) δ 7.0 (m, 3H), 4.4 (m, 3H), 3.0 (m, 2H), 2.5 (m, 3H), 1.38 (s, 3H), 1.28 (s, 6H). ¹³C{¹H} NMR (CDCl₃) δ 176.00, 175.17, 157.13, 156.62, 138.93, 135.45, 135.06, 128.55, 128.42, 128.22, 127.58, 127.18, 123.62, 118.64, 118.01, 117.64, 117.26, 82.66, 57.31, 56.28, 45.36, 44.36, 32.06, 31.24, 27.68, 25.85. ESI-MS (m/z): 384[M−H]⁻. Anal. Calcd. for C₁₆H₂₈N₄O₇S.1H₂O: C, 45.70; H, 6.71; N, 13.32. Found: C, 45.08; H, 6.62; N, 13.12.

### Example 44

### (S)-3-Methylcarbamoyl-7-sulfoamino-3,4-dihydro-1H-isoquinoline-2-carboxylic Acid Tert-Butyl Ester

7-amino-3-methylcarbamoyl-3,4-dihydro-1H-isoquinoline-2-carboxylic acid tert-butyl ester (0.313 g, 1.03 mmol) is dissolved in dimethylacetamide (4 mL) and to this is added sulfur trioxide pyridine complex (Aldrich, 0.2 g, 1.26 mmol). The reaction is allowed to stir for 4.5 h. at which point and aqueous solution of NH₄CO₃ (4 g per 100 mL, 25 mL) is added (pH8) and the aqueous layer is extracted with EtOAc (4×25 mL). The aqueous layer is collected and evaporated to give an off-white solid. RP-HPLC purified the crude solid. Yield 0.130 g (31%).

Mixture of rotational isomers. ¹H NMR (D₂O) δ 7.19 (d, 1H), 7.11 (d, 1H), 7.08 (s, 1H), 4.64-4.43 (m, 3H), 3.16-2.98 (m, 2H), 2.63 (s, 1.5H), 2.55 (s, 1.5H), 1.52 (s, 4.5H), 1.42 (s, 4.5H). ¹³C{¹H} NMR(D₂O) δ 175.9, 175.1, 157.1, 156.6, 138.9, 135.4, 135.0, 128.5, 128.4, 128.2, 118.6, 117.2, 82.6, 57.4, 56.3, 45.5, 44.5, 31.4, 31.2, 27.9, 27.8, 25.9. ESI-MS(m/z): 384[M−H]⁻; Anal. Calcd. For C₁₆H₂₈N₄O₇S.H₂O: C, 45.70; H, 6.67; N, 13.32. Found: C, 45.65; H, 6.93; N, 13.03.

### Examples 45-48

The following chemical formula along with Table 5 shows the structure of compounds made according to the description in Examples 45-48below:

TABLE 5

| | |
|---|---|
| | Formula (VII) |
| 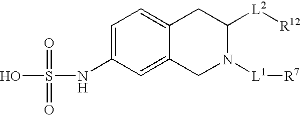 | |

| EXAMPLE | * | L¹ | L² | R⁷ | R¹² |
|---|---|---|---|---|---|
| 45 | | —CO₂— | —CO₂— | 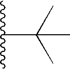 | —CH₃ |
| 46 | | —CO₂— | Covalent bond | 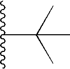 | 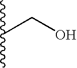 |
| 47 | | —CO₂— | 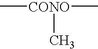 | 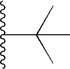 | —CH₃ |
| 48 | | —CO₂— | —CO₂— | 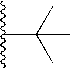 | 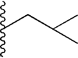 |

### Example 45

### (S)-7-Nitro-3,4-dihydro-1H-isoquinoline-2,3-dicarboxylic acid-2-tert-butyl ester-3-methyl Ester

(S)-7-Nitro-3,4-dihydro-1H-isoquinoline-2,3-dicarboxylic acid-2-tert-butyl ester is dissolved in 3:1 EtOAc/methanol (4 mL) and cooled to 0° C. To this stirring, cooled solution is slowly added trimethylsilyl diazomethane (2.0 M solution in Hexanes, Aldrich, 0.35 mL). An additional portion of TMS-diazomehtane (0.350 mL) is added until the evolution of gas had subsided. Acetic acid (0.1 mL) is added and the orange solution is concentrated to give an oily solid. Flash column chromatography eluting with 4:1 hexanes/EtOAc yielded a yellow fluffy solid. Yield: 0.157 g (70%). ¹H NMR (CDCl₃) δ 8.06 (d, 2H), 7.35 (d, 1H), 5.25 (m, 0.5H), 4.9 (m, 0.5H), 4.8-4.4 (m, 2H), 3.66 (d, 3H), 3.4-3.1 (m, 2H), 1.44 (m, 9H). ESI-MS (m/z): 337[M+H]⁺.

### (S)-7-Amino-3,4-dihydro-1H-isoquinoline-2,3-dicarboxylic acid-2-tert-butyl ester-3-methyl Ester

(S)-7-Nitro-3,4-dihydro-1H-isoquinoline-2,3-dicarboxylic acid-2-tert-butyl ester-3-methyl ester (0.157 g, 0.46 mmol) is dissolved in EtOAc (5 mL) and degassed by bubbling Argon through the reaction solution for 5 min. 10% Palladium on carbon (Aldrich, 0.05 g) is introduced into the vessel and the solution is again degassed. The vessel is placed under and atmosphere of H₂ at 40 psi for 4 h. The reaction is monitored by LC/MS and once complete, the solution is filtered through Celite and concentrated to give a yellow solid. Yield 0.123 g (88%).

Mixture of rotational isomers. ¹H NMR (CDCl₃, 8): 6.95 (d, 1H), 6.55 (d, 1H), 6.45 (s, 1H), 5.10 (t, 0.5H), 4.75 (t, 0.5H), 4.65 (dd, J=Hz, 1H), 4.45 (t, J=Hz, 1H), 3.67 (s, 1.5H), 3.65 (s, 1.5H), 3.1 (m, 2H), 1.55 (s, 4.5H), 1.48 (s, 4.5H). 13C{H} NMR (CDCl₃, 8): 172.6, 172.1, 155.5, 154.8, 145.1, 144.9, 134.9, 133.8, 129.2, 128.5, 122.1, 121.9, 114.2, 113.9, 112.7, 112.4, 80.4, 54.7, 52.8, 52.1, 52.0, 44.6, 44.0, 30.8, 30.3, 28.4, 28.3. ESI-MS (m/z): 307[M+H]⁺.

### (S)-7-Sulfoamino-3,4-dihydro-1H-isoquinoline-2,3-dicarboxylic acid-2-tert-butyl ester-3-methyl Ester

(S)-7-Amino-3,4-dihydro-1H-isoquinoline-2,3-dicarboxylic acid-2-tert-butyl ester-3-methyl ester (7) (0.103 g) is dissolved in pyridine (1 mL) and to this pinkish solution is added sulfur trioxide pyridine complex (0.05 g) and the solution is allowed to stir for 18 hr. Additional sulfur trioxide pryidine complex (0.06 g) is added and the reaction stirred for an additional 2 h. The mixture is diluted with aqueous NH₄CO₃ solution to pH 8 (ca. 10 mL) and evaporated to dryness. RP-HPLC purified the crude pink solid. Yield 0.091 g (66%).

Mixture of rotational isomers. ¹H NMR (D₂O) δ 7.02 (d, 1H), 6.91 (m, 2H), 4.8 (t, 0.5H), 4.50-4.30 (m, 3H), 3.52 (s, 1.5H), 3.05-2.90 (m, 2H), 1.37 (s, 4.5H), 1.28 (s, 4.5H). ¹³C{¹H} NMR (H₂O) δ 175.2, 174.6, 157.1, 156.6, 139.0, 134.7, 134.2, 128.9, 128.6, 127.3, 126.9, 118.3, 116.9, 82.8, 55.3, 54.0, 53.0, 44.9, 44.1, 30.3, 30.2, 27.8, 27.7. ESI-MS (m/z): 385[M−H]⁻; Anal. Calcd. for C₁₆H₂₅N₃O₇S.1/4H₂O: C, 47.11; H, 6.30; N, 10.30. Found: C, 47.22; H, 6.43; N, 10.07.

### Example 46

### (S)-3-Hydroxymethyl-7-sulfoamino-3,4-dihydro-1H-isoquinoline-2-carboxylic Acid Tert-Butyl Ester

(S)-3-Hydroxymethyl-7-amino-3,4-dihydro-1H-isoquinoline-2-carboxylic acid tert-butyl ester (0.1 g, 0.36 mmol) is dissolved in pyridine and to this is added sulfur trioxide pyridine complex (0.172 g, 1.1 mmol). The reaction is stirred for 20 min then quenched with 7% ammonium hydroxide solution (aq) (20 mL). Is evaporated to dryness and purified by RP-HPLC. Yield 0.05 g (37%).

Mixture of rotational isomers. ¹H NMR (D₂O) δ 6.7 (d, 1H), 6.57 (d, 1H), 6.50 (s, 1H), 4.48 (br, 2H), 4.06 (d, 1H), 3.76 (m, 2H), 2.76 (dd, 1H), 2.56 (d, 1H), 1.35 (s, 9H).). ¹³C{¹H} NMR (D₂O) δ 157.27, 143.93, 133.78, 129.96, 124.07, 116.15, 114.33, 82.38, 67.63, 67.32, 50.22, 48.89, 43.44, 42.90, 28.652, 28.03. ESI-MS(m/z): 357[M−H]. Anal. Calcd. For C₁₅H₂₂N₂O₆S.9/10H₂O.9/10NH₃: C, 46.20; H, 6.85; N, 10.42. Found: C, 45.97; H, 6.32; N, 10.48.

### Example 47

### (S)-3-(Methoxy-methyl-carbamoyl)-7-sulfoamino-3,4-dihydro-1H-isoquinoline-2-carboxylic Acid Tert-Butyl Ester

(S)-7-Nitro-3,4-dihydro-1H-isoquinoline-2,3-dicarboxylic acid-2-tert-butyl ester (2.0 g, 6.2 mmol), N-hydroxy benzotriazole (1.9 g, 12.4 mmol) and N-methoxy-methylamine hydrochloride (0.786 g, 8.1 mmol) are dissolved in DMF (20 mL) and N-methylmorpholine (2.7 mL, 25 mmol) is added. The reaction is cooled to 0° C. and EDCI (1.3 g, 6.82 mmol) is added. The reaction is allowed to stir overnight while warming to room temperature. The reaction is diluted with H₂O (250 mL) and extracted with EtOAc (2×100 mL). The organic layer is washed with brine (2×100 mL), dried over magnesium sulfate and concentrated. Is purified by flash column chromatography eluting with 7:3 hexanes/ethyl acetate. Yield 1.8 g (82%) ESI-MS(m/z): 366[M+H]⁺. 3-(Methoxy-methyl-carbamoyl)-7-nitro-3,4-dihydro-1H-isoquinoline-2,3-dicarboxylic acid-2-tert-butyl ester (0.30 g, 0.82 mmol) is dissolved in 1,4-dioxane and 10% palladium on carbon is added under N₂. The reaction is placed on the Parr apparatus and hydrogenated at 40 psi for 4 h. Reaction is filtered through celite and concentrated to give a clear slightly brown residue. Is purified by flash column eluting with first 24:1 chloroform/methanol then 19:1 chloroform/methanol. Further purification by RP-HPLC is required to give the product as a white residue. Yield 0.160 g (58%) ESI-MS(m/z): 336[M+H]⁺. 3-(Methoxy-methyl-carbamoyl)-7-amino-3,4-dihydro-1H-isoquinoline-2,3-dicarboxylic acid-2-tert-butyl ester (0.160 g, 0.48 mmol) is dissolved in pyridine (2.5 mL) and to this is added sulfur trioxide pyridine complex (0.228 g, 1.43 mmol). Reaction is stirred for 15 min then quenched with 7% ammonium hydroxide solution (aq) (20 mL). Is evaporated to dryness and purified by RP-HPLC. Yield 0.120 g (58%).

Mixture of rotational isomers. ¹H NMR (D₂O) δ 7.0 (br, 3H), 4.8 (br, 1H), 4.24.5 (m, 2H), 3.7 (br, 3H), 2.7-3.3 (m, 5H), 1.5 (s, 3H), 1.4 (s, 6H).). ¹³C{¹H} NMR (D₂O) δ 174.94, 173.26, 156.70, 156.42, 139.29, 136.00, 135.60, 128.57, 128.31, 127.93, 127.36, 118.55, 116.94, 82.88, 82.56, 68.07, 61.97, 53.80, 52.78, 45.60, 44.74, 35.98, 32.47, 30.19, 28.13. ESI-MS(m/z): 414[M−H]⁻; Anal. Calcd. For C₁₇H₂₈N₄O₇S.3/4H₂O: C, 45.93; H, 6.65; N, 12.60. Found: C, 46.00; H, 6.64; N, 12.57.

### Example 48

### (S)-7-Sulfoamino-3,4-dihydro-1H-isoquinoline-2,3-dicarboxylic acid 2-tert-butyl ester 3-isobutyl Ester

(S)-7-Nitro-3,4-dihydro-1H-isoquinoline-2,3-dicarboxylic acid 2-tert-butyl ester 3-isobutyl ester (2.3 g, 6.1 mmol) is dissolved in ethanol (20 mL) under nitrogen and 10% Pd/C (0.25 g) is added to this stirring solution. The resulting slurry is stirred under an atmosphere of hydrogen for 18 h. The slurry is filtered through Celite, concentrated and vacuum dried. The dry, crude amine is dissolved in dry pyridine and to this is added sulfur trioxide pyridine complex (2.86 g, 18 mmol). The homogeneous reaction is stirred for 5 min before adding a 7% NH₄OH/H₂O solution (20 mL). All volatiles are removed and the crude sulfamic acid is vacuum dried for 18 h. before purifying by RP-HPLC. Yield 0.500 g (19%-2 steps).

Mixture of rotational isomers. ¹H NMR (D₂O) δ 7.1 (m, 1H), 6.9-7.0 (m, 2H), 4.7 (m, 1H), 4.6-4.4 (m, 2H), 3.7 (m, 2H), 3.1 (m, 2H), 1.8 (m, 1H), 1.43 (s, 3H), 1.34 (s, 6H), 0.7 (m, 6H). ESI-MS (m/z): 428[M−H]⁻. Anal. Calcd. For C₁₉H₂₈N₂O₇S.1 NH₃.1/6H₂O: C, 50.88; H, 7.04; N, 9.37. Found: C, 50.85; H, 6.79; N, 9.19.

### Examples 49-50

### Example 49

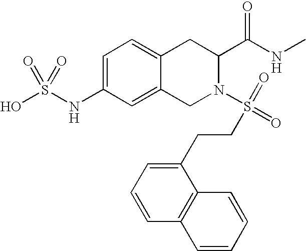

### {3-Methylcarbamoyl-2-[3-(naphthalene-1-sulfonyl)-propionyl]-1,2,3,4-tetrahydro-isoquinolin-7-yl}-sulfamic Acid

(S)-3-Methylcarbamoyl-7-nitro-1,2,3,4-tetrahydro-isoquinoline hydrochloride (0.20 g, 0.74 mmol) is slurried in dichloromethane (3 mL) and diisopropyl ethylamine (0.322 mL, 1.8 mmol) is added to the stirring solution. 2-(1-Naphthyl)ethanesulfonyl chloride (0.215, 0.8 mmol) is added and the reaction is stirred for 2 h. Ethyl acetate (20 mL) is added and the solution is washed with 1N sodium hydroxide (3×10 mL), water (10 mL), 0.1N hydrochloric acid (3×10 mL) and brine (10 mL). The organic layer is dried over magnesium sulfate, filtered and evaporated to give a yellow solid. Yield 0.257 g (77%). ESI-MS (m/z): 452[M−H]⁻. The product is dissolved in ethanol (3 mL) and degassed with argon for 2 min. 10% Palladium on carbon (0.08 g) is then added and hydrogen is bubbled through the stirring solution for 3 h. The reaction mixture is filtered through celite and the solvent is evaporated to give a yellow solid. Yield 0.229 g (95%). ESI-MS (m/z): 423[M−H]⁻. The product is dissolved in pyridine (3 mL) and sulfur trioxide pyridine complex (0.337 g, 1.2 mmol) is added to the stirring solution. The resulting heterogeneous solution is stirred for 5 min then quenched with 7% ammonium hydroxide (aq) (25 mL) and the solution is stirred for an additional 5 min. The solvents are evaporated to dryness and the residue is re-dissolved in 7% ammonium hydroxide (aq) (25 mL) and evaporated to dryness. The resulting solid is HPLC purified to give 0.105 g (37%) (27% over 3 steps) of an off-white solid. ¹H NMR (D₂O) δ 7.20-6.52 (m, 10H), 4.05 (m, 3H), 3.72 (m, 2H), 2.67 (m, 4H), 2.35 (s, 3H). ¹³C {¹H} NMR (D₂O) δ 173.6, 173.5, 139.5, 133.7, 133.6, 133.5, 131.0, 129.3, 129.0, 127.7, 126.7, 126.1, 125.9, 123.0, 118.6, 116.7, 56.5, 50.4, 45.5, 30.4, 26.2, 25.9. ESI-MS (m/z): 502[M−H]⁻. Anal. Calcd. for C₂₄H₂₈N₄O₇S₂.1/2H₂O: C, 51.69; H, 5.24; N, 10.05. Found: C, 51.88; H, 5.54; N, 10.37.

### Example 50

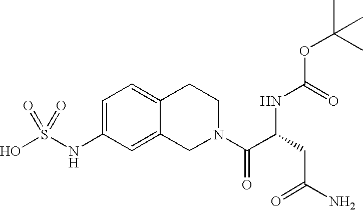

### R-[1-Carbamoylmethyl-2-oxo-2(7-sulfoamino-3,4-dihydro-1H-isoquinolin-2-yl)-ethyl]-carbamic Acid Tert Butyl Ester

7-Nitro-1,2,3,4-tetrahydro isoquinoline (0.3g, 0.1.67 mmol) is dissolved in 4:1 dichloromethane/DMF (10 mL) and diisopropylethyl amine (0.3 mL, 0.167 mmol) is added. To this is added Boc-D-Asn-OPNP (0.59 g, 1.67 mmol) and the reaction is allowed to stir for 18 h. The reaction is diluted with dichloromethane (75 mL) and washed with sat. aq. Sodium bicarbonate solution until the yellow color (p-nitrophenol) has dissipated then with brine (2×25 mL). The crude product is dried over magnesium sulfate and concentrated before final purification by flash column chromatography on silica gel eluting with 9:1 chloroform/methanol. Yield 0.225 g (34%) (orange-yellow solid) MS m/z 392[M+H]⁺.

R-[1-Carbamoylmethyl-2-(7-nitro-3,4-dihydro-1H-isoquinolin-2-yl)-2-oxo-ethyl]-carbamic acid tert-butyl ester (0.225 g, 0.574 mmol) is dissolved in ethyl acetate (4 mL) and degassed with argon for several minutes. 10% Palladium on carbon (0.05 g) is added and hydrogen is introduced into the flask via balloon. The reaction is agitated for 3 h before filtering through celite and concentrating to give an yellow residue. Yield 0.16 g.

R-[1-Carbamoylmethyl-2-(7-amino-3,4-dihydro-1H-isoquinolin-2-yl)-2-oxo-ethyl]-carbamic acid tert-butyl ester (0.16 g, 0.44 mmol) is dissolved in pyridine (1.5 mL) and sulfur trioxide pyridine complex (0.21 g, 1.32 mmol) is added in one portion. The reaction is allowed to stir for 4 min then quenched with 7% ammonium hydroxide (aq) (20 mL). The reaction is concentrated and purified by RP-HPLC. Yield 0.050g (25%).

Mixture of rotational isomers: ¹H NMR (D₂O) δ 7.1 (d, 1H), 6.9 (m, 2H), 4.8 (br, 1H), 4.6-4.3 (m, 2H), 3.8-3.5 (m, 2H), 2.8-2.4 (m, 4H), 1.3 (d, 9H). MS m/z 441[M−H]⁻. Anal. Calcd. for C₁₈H₂₉N₅O₇S: C, 46.14; H, 6.45; N, 14.95. Found: C, 46.44; H, 6.27; N, 14.94.

### Example A

A tablet composition for oral administration, according to the present invention, is made comprising:

| Component | Amount |
|---|---|
| Example 1 compound | 150 mg |
| Lactose | 120 mg |
| Maize Starch | 70 mg |
| Talc | 4 mg |
| Magnesium Stearate | 1 mg |

### Example B

A capsule containing 200 mg of active for oral administration, according to the present invention, is made comprising:

| Component | Amount (% w/w) |
|---|---|
| Example 2 compound | 15% |
| Hydrous Lactose | 43% |
| Microcrystalline Cellulose | 33% |
| Crosspovidone | 3.3% |
| Magnesium Stearate | 5.7% |

Other subject compounds are used with substantially similar results.

Except as otherwise noted, all amounts including quantities, percentages, portions, and proportions, are understood to be modified by the word “about”, and amounts are not intended to indicate significant digits.

Except as otherwise noted, the articles “a”, “an”, and “the” mean “one or more”.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope.

1. A compound according to formula I: 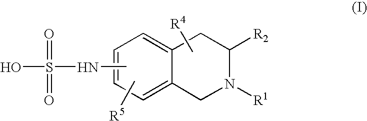
wherein:
A) R¹ is -L¹-[C(R^{6a}R^{6b})]ₘR⁷, wherein:
a) L1 is selected from the group consisting of covalent bond, —O—, —S—, —N—, —CO₂—, —CO—, —OCO₂—, —SO—, —SO₂—, —CSN(R⁸)—, —CON(R⁸)O—, —CON(R⁸)—, —OCON(R⁸)—; wherein R⁸ is hydrogen or substituted or unsubstituted C₁-C₅ alkyl;
b) R^{6a} and R^{6b} are each independently selected from the group consisting of hydrogen, —OR⁹, —N(R⁹)₂, —CO₂R⁹, —CON(R⁹)₂, —NHCOR⁹, —NHCO₂R⁹, ═NR⁹, —R⁹, and mixtures thereof; wherein each R⁹ is independently selected from the group consisting of hydrogen, substituted or unsubstituted C₁-C₅ alkyl, and substituted or unsubstituted aryl or alkylenearyl; or two R⁹ units can be taken together to form a substituted or unsubstituted carbocyclic or heterocyclic ring comprising from 3 to 7 atoms;
c) m is an index selected from 0 to 5;
d) R is selected from the group consisting of nil, hydrogen, substituted or unsubstituted C₁-C₁₀ alkyl, substituted or unsubstituted C₁-C₁₀ heteroalkyl, substituted or unsubstituted hydrocarbyl, substituted or unsubstituted heterocyclyl, substituted or unsubstituted aryl or alkylenearyl, substituted or unsubstituted heteroaryl or alkyleneheteroaryl; or
e) R⁷ and a R⁹ can be taken together to form a substituted or unsubstituted carbocyclic or heterocyclic ring comprising from 3 to 7 atoms;
B) R² is —(CH₂)ⱼ-L²-[C(R^{11a}R^{11b})]_{g}R¹², wherein:
a) j is an index selected from 0 to 5;
b) L² is selected from the group consisting of covalent bond, —O—, —S—, —N—, —CO₂—, —CO—, —OCO₂—, —SO—, —SO₂—, —CSN(R¹⁰)—, —CON(R¹⁰)—, —CON(R¹⁰)O—, —OCON(R¹⁰)—; wherein R¹⁰ is hydrogen or substituted or unsubstituted C₁-C₅ alkyl;
c) R^{11a} and R^{11b} are each independently selected from the group consisting of hydrogen, —OR¹³, —N(R¹³)₂, —CO₂R¹³, —CON(R¹³)₂, —NHCOR¹³, —NHCO₂R¹³, ═NR¹³, —R¹³, and mixtures thereof; wherein each R¹³ is independently selected from the group consisting of hydrogen, substituted or unsubstituted C₁-C₅ alkyl, and substituted or unsubstituted aryl or alkylenearyl; or two R¹³ units can be taken together to form a substituted or unsubstituted carbocyclic or heterocyclic ring comprising from 3 to 7 atoms;
d) g is an index selected from 0 to 5;
e) R¹² is selected from the group consisting of nil, hydrogen, substituted or unsubstituted C₁-C₁₀ alkyl, substituted or unsubstituted hydrocarbyl, substituted or unsubstituted heterocyclyl, substituted or unsubstituted aryl or alkylenearyl, substituted or unsubstituted heteroaryl or alkyleneheteroaryl; or
f) R¹² and a R¹³ can be taken together to form a substituted or unsubstituted carbocyclic or heterocyclic ring comprising from 3 to 7 atoms; and
C) R⁴ and R⁵ are each independently selected from hydrogen or substitution unit.

2. The compound of claim 1 having the formula (II):

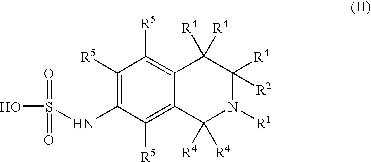

3. The compound of claim 2, wherein:
a) j is 0; and
b) L² is —CON(R⁸)—; and
c) R^{6a} and R^{6b} are each hydrogen.

4. The compound of claim 3, wherein L¹ is selected from the group consisting of —CO—, —CO₂—, and SO₂—.

5. The compound of claim 4, wherein R⁷ is substituted or unsubstituted phenyl.

6. The compound of claim 2, wherein L¹ is selected from —CO—, —CO₂—, —CONH—, and —SO₂—.

7. The compound of claim 6, wherein R² is hydrogen.

8. The compound of claim 2, wherein
a) R² is hydrogen; and
b) L¹ is —CO—

9. The compound of claim 8, wherein:
a) R^{6a} and R^{6b} are each hydrogen; and
b) m is an index from 1-5.

10. The compound of claim 9, wherein R⁷ substituted or unsubstituted phenyl.

11. The compound of claim 2, wherein:
a) j is o; and
b) L² is —CON(R⁸)—.

12. The compound of claim 11, wherein:
a) L¹ is selected from —CO—, and —CO₂—; and
b) R⁷ is substituted or unsubstited C₁-C₁₀ alkyl.

13. The compound of claim 2, wherein:
a) L¹ is —CO₂—; and
b) R⁷ is substituted or unsubstituted C₁-C₁₀ alkyl.

14. The compound of claim 13, wherein:
a) j is 0; and
b) L² is selected from covalent bond, —CO₂—, and —CON(CH₃)O—

15. The compound of claim 14, wherein R¹² is selected from substituted or unsubstituted C₁-C₁₀ alkyl.

16. The compound of claim 1, wherein the compound is selected from the group consisting of:
(S)-3-Methylcarbamoyl-7-sulfoamino-3,4-dihydro-1H-isoquinoline-2-carboxylic acid tert-butyl ester; [(S)-3-Methylcarbamoyl-2-(3-phenyl-propionyl)-1,2,3,4-tetrahydro-isoquinolin-7-yl]-sulfamic acid; [(S)-2-Benzylcarbamoyl-3-methylcarbamoyl-1,2,3,4-tetrahydro-isoquinolin-7-yl]-sulfamic acid; (S)-(3-Methylcarbamoyl-2-phenylmethanesulfonyl-1,2,3,4-tetrahydro-isoquinolin-7-yl)-sulfamic acid; (S)-{3-Methylcarbamoyl-2-[3-(4-trifluoromethyl-phenyl)-propionyl]-1,2,3,4-tetrahydro-isoquinolin-7-yl}-sulfamic acid; (S)-[3-Methylcarbamoyl-2-(4-phenyl-butyryl)-1,2,3,4-tetrahydro-isoquinolin-7-yl]-sulfamic acid; (S)-[3-Methylcarbamoyl-2-(5-phenyl-pentanoyl)-1,2,3,4-tetrahydro-isoquinolin-7-yl]-sulfamic acid; (S)-{3-Methylcarbamoyl-2-[3-(3-sulfoamino-phenyl)-propionyl]-1,2,3,4-tetrahydro-isoquinolin-7-yl}-sulfamic acid; (S)-[3-Methylcarbamoyl-2-(3-p-tolyl-propionyl)-1,2,3,4-tetrahydro-isoquinolin-7-yl]-sulfamic acid; (S)-{2-[3-(3-Hydroxy-phenyl)-propionyl]-3-methylcarbamoyl-1,2,3,4-tetrahydro-isoquinolin-7-yl}-sulfamic acid; (S)-{2-[3-(4-Methoxy-phenyl)-propionyl]-3-methylcarbamoyl-1,2,3,4-tetrahydro-isoquinolin-7-yl}-sulfamic acid; (S)-[3-Benzylcarbamoyl-2-(4-propyl-benzoyl)-1,2,3,4-tetrahydro-isoquinolin-7-yl]-sulfamic acid; (S)-[3-Benzylcarbamoyl-2-(3-phenyl-propionyl)-1,2,3,4-tetrahydro-isoquinolin-7-yl]-sulfamic acid; (S)-3-Benzylcarbamoyl-7-sulfoamino-3,4-dihydro-1H-isoquinoline-2-carboxylic acid benzyl ester; (S)-{3-Methylcarbamoyl-2-[3-(3-sulfamoyl-phenyl)-propionyl]-1,2,3,4-tetrahydro-isoquinolin-7-yl}-sulfamic acid; (S)-{2-[3-(3-Acetylsulfamoyl-phenyl)-propionyl]-3-methylcarbamoyl-1,2,3,4-tetrahydro-isoquinolin-7-yl}-sulfamic acid; (S)-{3-Methylcarbamoyl-2-[3-(3-propionylsulfamoyl-phenyl)-propionyl]-1,2,3,4-tetrahydro-isoquinolin-7-yl}-sulfamic acid; (S)-(2-{3-[3-(2,2-Dimethyl-propionylsulfamoyl)-phenyl]-propionyl}-3-methylcarbamoyl-1,2,3,4-tetrahydro-isoquinolin-7-yl)-sulfamic acid; (S)-{2-[3-(3-Benzoylsulfamoyl-phenyl)-propionyl]-3-methylcarbamoyl-1,2,3,4-tetrahydro-isoquinolin-7-yl}-sulfamic acid; (S)-{3-Methylcarbamoyl-2-[3-(4-sulfamoyl-phenyl)-propionyl]-1,2,3,4-tetrahydro-isoquinolin-7-yl}-sulfamic acid; (S)-{2-[3-(4-Acetylsulfamoyl-phenyl)-propionyl]-3-methylcarbamoyl-1,2,3,4-tetrahydro-isoquinolin-7-yl}-sulfamic acid; (R)-{3-Methylcarbamoyl-2-[3-(3-sulfamoyl-phenyl)-propionyl]-1,2,3,4-tetrahydro-isoquinolin-7-yl}-sulfamic acid; (R)-{2-[3-(3-Acetylsulfamoyl-phenyl)-propionyl]-3-methylcarbamoyl-1,2,3,4-tetrahydro-isoquinolin-7-yl}sulfamic acid; (S)-3-[3-(3-Methylcarbamoyl-7-sulfoamino-3,4-dihydro-1H-isoquinolin-2-yl)-3-oxo-propyl]-benzoic acid; (S)-{2-[3-(3-Acetylsulfamoyl-phenyl)-propionyl]-3-phenethylcarbamoyl-1,2,3,4-tetrahydro-isoquinolin-7-yl}-sulfamic acid; (S)-(2-Benzoyl-3-methylcarbamoyl-1,2,3,4-tetrahydroisoquinolin-7-yl)-sulfamic acid; (S)-{2-[3-(3-Chloro-phenyl)-propionyl]-3-methylcarbamoyl-1,2,3,4-tetrahydro-isoquinolin-7-yl}-sulfamic acid; 7-Sufoamino-3,4-dihydro-1H-isoquinoline-2-carboxylic acid tert-butyl ester; 7-Sulfoamino-3,4-dihydro-1H-isoquinoline-2-carboxylic acid benzyl ester; 2-(Benzylcarbamoyl-1,2,3,4-tetrahydro-isoquinolin-7-yl)-sulfamic acid; [2-(3-Phenyl-propionyl)-1,2,3,4-tetrahydro-isoquinolin-7-yl]-sulfamic acid; {2-[2-(1-methyl-1H-indol-3-yl)-acetyl]-1,2,3,4-tetrahydro-isoquinolin-7-yl}-sulfamic acid; 2-(Phenylmethanesulfonyl-1,2,3,4-tetrahydro-isoquinolin-7-yl)-sulfamic acid; 4-Oxo-4(7-sulfoamino-3,4-dihydro-1H-isoquinolin-2-yl)-butyric acid; {2-[3-(3-Sulfoamino-phenyl)-propionyl]-1,2,3,4-tetrahydro-isoquinolin-7-yl}-sulfamic acid; {2-[3-(3-Acetylsulfamoyl-phenyl)-propionyl]-1,2,3,4-tetrahydro-isoquinolin-7-yl}-sulfamic acid; {2-[3-(3-Sulfamoyl-phenyl)-propionyl]-1,2,3,4-tetrahydro-isoquinolin-7-yl}-sulfamic acid; (S)-4-(3-Methylcarbamoyl-7-sulfoamino-3,4-dihydro-1H-isoquinolin-2-yl)₄-oxo-butyric acid; (S)-3-Phenethylcarbamoyl-7-sulfoamino-1,2,3,4-tetrahydro-naphthalene-2-carboxylic acid tert-butyl ester; (S)-3-Ethylcarbamoyl-7-sulfoamino-1,2,3,4-tetrahydro-naphthalene-2-carboxylic acid tert-butyl ester; (S)-(3-Benzylcarbamoyl-2-hexanoyl-1,2,3,4-tetrahydro-isoquinolin-7-yl)-sulfamic acid; (S)-3-Benzylcarbamoyl-7-sulfoamino-3,4-dihydro-1H-isoquinoline-2-carboxylic acid tert-butyl ester; (R)-7-Nitro-1,2,3,4-tetrahydro-isoquinoline-3-carboxylic acid; (S)-3-Methylcarbamoyl-7-sulfoamino-3,4-dihydro-1H-isoquinoline-2-carboxylic acid tert-butyl ester; (S)-7-Nitro-3,4-dihydro-1H-isoquinoline-2,3-dicarboxylic acid-2-tert-butyl ester-3-methyl ester; (S)-3-Hydroxymethyl-7-sulfoamino-3,4-dihydro-1H-isoquinoline-2-carboxylic acid tert-butyl ester; (S)-3-(Methoxy-methyl-carbamoyl)-7-sulfoamino-3,4-dihydro-1H-isoquinoline-2-carboxylic acid tert-butyl ester; (S)-3-(Methoxy-methyl-carbamoyl)-7-sulfoamino-3,4-dihydro-1H-isoquinoline-2-carboxylic acid tert-butyl ester; (S)-7-Sulfoamino-3,4-dihydro-1H-isoquinoline-2,3-dicarboxylic acid 2-tert-butyl ester 3-isobutyl ester; {3-Methylcarbamoyl-2-[3-(naphthalene-1-sulfonyl)-propionyl]-1,2,3,4-tetrahydro-isoquinolin-7-yl}-sulfamic acid; and R-[1-Carbamoylmethyl-2-oxo-2(7-sulfoamino-3,4-dihydro-1H-isoquinolin-2-yl)-ethyl]-carbamic acid tert butyl ester.

17. A method of treating a protein tyrosine phosphatase (PTPase) mediated disorder comprising administering a compound of claim 1 to a subject in need thereof.

18. The method of claim 17, wherein the disorder is selected from the group consisting of atherosclerotic cardiovascular disease including peripheral vascular disease, coronary disease and cerebral vascular disease; heart failure; hypertension; diabetes (Type 1 and Type 2); skeletal muscle atrophy; osteoporosis; obesity; disorders of the gastrointestinal tract including inflammatory bowel disease and ulcer; wound healing and wrinkle repair/prevention; hair loss and cancer.

19. A pharmaceutical composition comprising:
a) safe and effective amount of a compound of claim 1;
b) a pharmaceutically-acceptable carrier.
